(19) United States
(12) Patent Application Publication
(10) Pub. No.: US 2003/0119731 A1
(43) Pub. Date: Jun. 26, 2003

(54) Omega-conopeptides

(75) Inventors: Baldomero M. Olivera, Salt Lake City, UT (US); J. Michael McIntosh, Salt Lake City, UT (US); Maren Watkins, Salt Lake City, UT (US); James E. Garrett, Salt Lake City, UT (US); Ki-Joon Shon, Shaker Heights, OH (US); Richard B. Jacobsen, San Francisco, CA (US); Robert M. Jones, Salt Lake City, UT (US); G. Edward Cartier, Salt Lake City, UT (US)

Correspondence Address: ROTHWELL, FIGG, ERNST & MANBECK, P.C., 1425 K STREET, N.W., SUITE 800, WASHINGTON, DC, 20005, US

(73) Assignee: UNIVERSITY OF UTAH RESEARCH FOUNDATION, Salt Lake City, UT (US)

(21) Appl. No.: 09/910,082

(22) Filed: Jul. 23, 2001

Related U.S. Application Data
(60) Provisional application No. 60/219,616, filed on Jul. 21, 2000.
(60) Provisional application No. 60/265,888, filed on Feb. 5, 2001.

(51) Int. Cl. A61K 38/17; C07K 14/435; C07H 21/04

(52) U.S. Cl. 514/12; 530/350; 536/23.5

(57) ABSTRACT

The invention relates to ω-conopeptides, derivatives or pharmaceutically acceptable salts thereof, and uses thereof, including the treatment of neurologic and psychiatric disorders, such as anticonvulsant agents, as neuroprotective agents, as cardiovascular agents or for the management of pain. The invention further relates to nucleic acid sequences encoding the conopeptides and encoding propeptides, as well as the propeptides.

### CROSS REFERENCE TO RELATED APPLICATIONS

The present application claims benefit under 35 USC §119(e) to U.S. provisional patent applications Ser. No. 60/219,616 filed on Jul. 21, 2000 and Ser. No. 60/265,888 filed on Feb. 5, 2001. Each of these applications are incorporated herein by reference.

This invention was made with Government support under Grant No. PO1 GM48677 awarded by the National Institute of General Medical Sciences, National Institutes of Health, Bethesda, Md. The United States Government has certain rights in the invention.

### BACKGROUND OF THE INVENTION

The invention relates to ω-conopeptides, derivatives or pharmaceutically acceptable salts thereof, and uses thereof, including the treatment of neurologic and psychiatric disorders, such as anticonvulsant agents, as neuroprotective agents, as cardiovascular agents or for the management of pain. The invention further relates to nucleic acid sequences encoding the conopeptides and encoding propeptides, as well as the propeptides.

The publications and other materials used herein to illuminate the background of the invention, and in particular, cases to provide additional details respecting the practice, are incorporated by reference, and for convenience are referenced in the following text by author and date and are listed alphabetically by author in the appended bibliography.

Conus is a genus of predatory marine gastropods (snails) which envenomate their prey. Venomous cone snails use a highly developed projectile apparatus to deliver their cocktail of toxic conotoxins into their prey. In fish-eating species such as Conus magus the cone detects the presence of the fish using chemosensors in its siphon and when close enough extends its proboscis and fires a hollow harpoon-like tooth containing venom into the fish. This immobilizes the fish and enables the cone snail to wind it into its mouth via an attached filament.

For general information on Conus and their venom see the website address http://grimwade.biochem.unimelb.edu.au/cone/referenc.html. Prey capture is accomplished through a sophisticated arsenal of peptides which target specific ion channel and receptor subtypes. Each Conus species venom appears to contain a unique set of 50-200 peptides. The composition of the venom differs greatly between species and between individual snails within each species, each optimally evolved to paralyse it's prey. The active components of the venom are small peptides toxins, typically 12-30 amino acid residues in length and are typically highly constrained peptides due to their high density of disulphide bonds.

The venoms consist of a large number of different peptide components that when separated exhibit a range of biological activities: when injected into mice they elicit a range of physiological responses from shaking to depression. The paralytic components of the venom that have been the focus of recent investigation are the α-, ω- and μ-conotoxins. All of these conotoxins act by preventing neuronal communication, but each targets a different aspect of the process to achieve this. The α-conotoxins target nicotinic ligand gated channels, the μ-conotoxins target the voltage-gated sodium channels and the ω-conotoxins target the voltage-gated calcium channels (Olivera et al., 1985; Olivera et al., 1990). For example a linkage has been established between α-, αA- &Φ-conotoxins and the nicotinic ligand-gated ion channel; ω-conotoxins and the voltage-gated calcium channel; μ-conotoxins and the voltage-gated sodium channel; δ-conotoxins and the voltage-gated sodium channel; κ-conotoxins and the voltage-gated potassium channel; conantokins and the ligand-gated glutamate (NMDA) channel.

However, the structure and function of only a small minority of these peptides have been determined to date. For peptides where function has been determined, three classes of targets have been elucidated: voltage-gated ion channels; ligand-gated ion channels, and G-protein-linked receptors.

Conus peptides which target voltage-gated ion channels include those that delay the inactivation of sodium channels, as well as blockers specific for sodium channels, calcium channels and potassium channels. Peptides that target ligand-gated ion channels include antagonists of NMDA and serotonin receptors, as well as competitive and noncompetitive nicotinic receptor antagonists. Peptides which act on G-protein receptors include neurotensin and vasopressin receptor agonists. The unprecedented pharmaceutical selectivity of conotoxins is at least in part defined by a specific disulfide bond frameworks combined with hypervariable amino acids within disulfide loops (for a review see McIntosh et al., 1998).

There are drugs used in the treatment of pain, which are known in the literature and to the skilled artisan. See, for example, Merck Manual, 16th Ed. (1992). However, there is a demand for more active analgesic agents with diminished side effects and toxicity and which are non-addictive. The ideal analgesic would reduce the awareness of pain, produce analgesia over a wide range of pain types, act satisfactorily whether given orally or parenterally, produce minimal or no side effects, be free from tendency to produce tolerance and drug dependence.

Due to the high potency and exquisite selectivity of the conopeptides, several are in various stages of clinical development for treatment of human disorders. For example, two Conus peptides are being developed for the treatment of pain. The most advanced is ω-conotoxin MVIIA (ziconotide), an N-type calcium channel blocker (see Heading, C., 1999;

U.S. Pat. No. 5,859,186). ω-Conotoxin MVIIA, isolated from Conus magus, is approximately 1000 times more potent than morphine, yet does not produce the tolerance or addictive properties of opiates. ω-Conotoxin MVIIA has completed Phase III (final stages) of human clinical trials and has been approved as a therapeutic agent. ω-Conotoxin MVIIA is introduced into human patients by means of an implantable, programmable pump with a catheter threaded into the intrathecal space. Preclinical testing for use in post-surgical pain is being carried out on another Conus peptide, contulakin-G, isolated from Conus geographus (Craig et al. 1999).

Contulakin-G is a 16 amino acid O-linked glycopeptide whose C-terminus resembles neurotensin. It is an agonist of neurotensin receptors, but appears significantly more potent than neurotensin in inhibiting pain in in vivo assays.

Ischemic damage to the central nervous system (CNS) may result form either global or focal ischemic conditions. Global ischemia occurs under conditions in which blood flow to the entire brain ceases for a period of time, such as may result from cardiac arrest. Focal ischemia occurs under conditions in which a portion of the brain is deprived of its normal blood supply, such as may result from thromboembolytic occlusion of a cerebral vessel, traumatic head or spinal cord injury, edema or brain or spinal cord tumors. Both global and focal ischemic conditions have the potential for widespread neuronal damage, even if the global ischemic condition is transient or the focal condition affects a very limited area.

Epilepsy is a recurrent paroxysmal disorder of cerebral function characterized by sudden brief attacks of altered consciousness, motor activity, sensory phenomena or inappropriate behavior caused by abnormal excessive discharge of cerebral neurons. Convulsive seizures, the most common form of attacks, begin with loss of consciousness and motor control, and tonic or clonic jerking of all extremities but any recurrent seizure pattern may be termed epilepsy. The term primary or idiopathic epilepsy denotes those cases where no cause for the seizures can be identified. Secondary or symptomatic epilepsy designates the disorder when it is associated with such factors as trauma, neoplasm, infection, developmental abnormalities, cerebrovascular disease, or various metabolic conditions. Epileptic seizures are classified as partial seizures (focal, local seizures) or generalized seizures (convulsive or nonconvulsive). Classes of partial seizures include simple partial seizures, complex partial seizures and partial seizures secondarily generalized. Classes of generalized seizures include absence seizures, atypical absence seizures, myoclonic seizures, clonic seizures, tonic seizures, tonic-clonic seizures (grand mal) and atonic seizures. Therapeutics having anticonvulsant properties are used in the treatment of seizures. Most therapeutics used to abolish or attenuate seizures act at least through effects that reduce the spread of excitation from seizure foci and prevent detonation and disruption of function of normal aggregates of neurons. Traditional anticonvulsants that have been utilized include phenytoin, phenobarbital, primidone, carbamazepine, ethosuximide, clonazepam and valproate. Several novel and chemically diverse anticonvulsant medications recently have been approved for marketing, including lamotrigine, ferlbamate, gabapentin and topiramate. For further details of seizures and their therapy, see Rall & Schleifer (1985) and The Merck Manual (1992).

In view of a large number of biologically active substances in Conus species it is desirable to further characterize them and to identify peptides capable of treating disorders involving voltage gated ion channels, such as stroke and pain. Surprisingly, and in accordance with this invention, Applicants have discovered novel conotoxins that can be useful for the treatment of disorders involving voltage gated ion channels and could address a long felt need for a safe and effective treatment.

### SUMMARY OF THE INVENTION

The present invention is directed to ω-conopeptides, derivatives or pharmaceutically acceptable salts thereof, and uses thereof, including the treatment of neurologic and psychiatric disorders, such as anticonvulsant agents, as neuroprotective agents, as cardiovascular agents or for the management of pain. The invention is further directed to nucleic acid sequences encoding the ω-conopeptides and encoding propeptides, as well as the propeptides.

More specifically, the present invention is directed to ω-conopeptides, having the amino acid sequences set forth in Table 2 below.

The present invention is also directed to derivatives or pharmaceutically acceptable salts of the ω-conopeptides or the derivatives. Examples of derivatives include peptides in which the Arg residues may be substituted by Lys, ornithine, homoargine, nor-Lys, N-methyl-Lys, N,N-dimethyl-Lys, N,N,N-trimethyl-Lys or any synthetic basic amino acid; the Lys residues may be substituted by Arg, ornithine, homoargine, nor-Lys, or any synthetic basic amino acid; the Tyr residues may be substituted with meta-Tyr, ortho-Tyr, nor-Tyr, mono-halo-Tyr, di-halo-Tyr, O-sulpho-Tyr, O-phospho-Tyr, nitro-Tyr or any synthetic hydroxy containing amino acid; the Ser residues may be substituted with Thr or any synthetic hydroxylated amino acid; the Thr residues may be substituted with Ser or any synthetic hydroxylated amino acid; the Phe residues may be substituted with any synthetic aromatic amino acid; the Trp residues may be substituted with Trp (D), neo-Trp, halo-Trp (D or L) or any aromatic synthetic amino acid; and the Asn, Ser, Thr or Hyp residues may be glycosylated. The halogen may be iodo, chloro, fluoro or bromo; preferably iodo for halogen substituted-Tyr and bromo for halogen-substituted Trp. The Tyr residues may also be substituted with the 3-hydroxyl or 2-hydroxyl isomers (meta-Tyr or ortho-Tyr, respectively) and corresponding O-sulpho- and O-phospho-derivatives. The acidic amino acid residues may be substituted with any synthetic acidic amino acid, e.g., tetrazolyl derivatives of Gly and Ala. The aliphatic amino acids may be substituted by synthetic derivatives bearing non-natural aliphatic branched or linear side chains CₙH₂ₙ₊₂up to and including n=8. The Cys residues may be in D or L configuration and may optionally be substituted with homocysteine (D or L).

Examples of synthetic aromatic amino acid include, but are not limited to, nitro-Phe, 4-substituted-Phe wherein the substituent is C₁-C₃ alkyl, carboxyl, hyrdroxymethyl, sulphomethyl, halo, phenyl, —CHO, —CN, —SO₃H and —NHAc. Examples of synthetic hydroxy containing amino acid, include, but are not limited to, such as 4-hydroxymethyl-Phe, 4-hydroxyphenyl-Gly, 2,6-dimethyl-Tyr and 5-amino-Tyr. Examples of synthetic basic amino acids include, but are not limited to, N-1-(2-pyrazolinyl)-Arg, 2-(4-piperinyl)-Gly, 2-(4-piperinyl)-Ala, 2-[3-(2S)pyrrolininyl)-Gly and 2-[3-(2S)pyrrolininyl)-Ala. These and other synthetic basic amino acids, synthetic hydroxy containing amino acids or synthetic aromatic amino acids are described in Building Block Index, Version 3.0 (1999 Catalog, pages 4-47 for hydroxy containing amino acids and aromatic amino acids and pages 66-87 for basic amino acids; see also http://www.amino-acids.com), incorporated herein by reference, by and available from RSP Amino Acid Analogues, Inc., Worcester, Mass. Examples of synthetic acid amino acids include those derivatives bearing acidic functionality, including carboxyl, phosphate, sulfonate and synthetic tetrazolyl derivatives such as described by Ornstein et al. (1993) and in U.S. Pat. No. 5,331,001, each incorporated herein by reference.

Optionally, in the ω-conopeptides of the present invention, the Asn residues may be modified to contain an N-glycan and the Ser, Thr and Hyp residues may be modified to contain an O-glycan (e.g., g—N, g—S, g—T and g—Hyp). In accordance with the present invention, a glycan shall mean any N—, S—or O-linked mono—, di—, tri—, poly—or oligosaccharide that can be attached to any hydroxy, amino or thiol group of natural or modified amino acids by synthetic or enzymatic methodologies known in the art. The monosaccharides making up the glycan can include D-allose, D-altrose, D-glucose, D-mannose, D-gulose, D-idose, D-galactose, D-talose, D-galactosamine, D-glucosamine, D-N-acetyl-glucosamine (GlcNAc), D-N-acetyl-galactosamine (GalNAc), D-fucose or D-arabinose. These saccharides may be structurally modified, e.g., with one or more O-sulfate, O-phosphate, O-acetyl or acidic groups, such as sialic acid, including combinations thereof. The gylcan may also include similar polyhydroxy groups, such as D-penicillamine 2,5 and halogenated derivatives thereof or polypropylene glycol derivatives. The glycosidic linkage is beta and 1-4 or 1-3, preferably 1-3. The linkage between the glycan and the amino acid may be alpha or beta, preferably alpha and is 1−.

Core O-glycans have been described by Van de Steen et al. (1998), incorporated herein by reference. Mucin type O-linked oligosaccharides are attached to Ser or Thr (or other hydroxylated residues of the present peptides) by a GalNAc residue. The monosaccharide building blocks and the linkage attached to this first GalNAc residue define the “core glycans,” of which eight have been identified. The type of glycosidic linkage (orientation and connectivities) are defined for each core glycan. Suitable glycans and glycan analogs are described further in U.S. Ser. No. 09/420,797 filed Oct. 19, 1999 and in PCT Application No. PCT/US99/24380 filed Oct. 19, 1999 (PCT Published Application No. WO 00/23092), each incorporated herein by reference. A preferred glycan is Gal(β1-3)GalNAc(α1→).

Optionally, in the ω-conopeptides described above, pairs of Cys residues may be replaced pairwise with isoteric lactam or ester-thioether replacements, such as Ser/(Glu or Asp), Lys/(Glu or Asp) or Cys/Ala combinations. Sequential coupling by known methods (Barnay et al., 2000; Hruby et al., 1994; Bitan et al., 1997) allows replacement of native Cys bridges with lactam bridges. Thioether analogs may be readily synthesized using halo-Ala residues commercially available from RSP Amino Acid Analogues.

The present invention is further directed to a method of treating disorders associated with voltage gated ion channel disorders in a subject comprising administering to the subject an effective amount of the pharmaceutical composition comprising a therapeutically effective amount of a ω-conopeptide described herein or a pharmaceutically acceptable salt or solvate thereof. The present invention is also directed to a pharmaceutical composition comprising a therapeutically effective amount of a ω-conopeptide described herein or a pharmaceutically acceptable salt or solvate thereof and a pharmaceutically acceptable carrier.

More specifically, the present invention is further directed to uses of these peptides or nucleic acids as described herein, including the treatment of neurologic disorders, such as anticonvulsant agents, as neuroprotective agents, such as for treating stroke, as cardiovascular agents or for the management of pain.

More specifically, the present invention is also directed to nucleic acids which encode conopeptides of the present invention or which encodes precursor peptides for these conopeptides, as well as the precursor peptide. The nucleic acid sequences encoding the precursor peptides of other conopeptides of the present invention are set forth in Table 1. Table 1 also sets forth the amino acid sequences of these precursor peptides.

### DETAILED DESCRIPTION OF THE PREFERRED EMBODIMENT

The present invention is to ω-conopeptides, derivatives or pharmaceutically acceptable salts thereof. The present invention is further directed to the use of this peptide, derivatives thereof and pharmaceutically acceptable salts thereof for the treatment of neurologic disorders, such as anticonvulsant agents, as neuroprotective agents, such as for treating stroke, as cardiovascular agents or for the management of pain, e.g. as analgesic agents. The invention is further directed to nucleic acid sequences encoding the ω-conopeptides and encoding propeptides, as well as the propeptides.

The present invention, in another aspect, relates to a pharmaceutical composition comprising an effective amount of an ω-conopeptides, a mutein thereof, an analog thereof, an active fragment thereof or pharmaceutically acceptable salts or solvates. Such a pharmaceutical composition has the capability of acting at voltage gated ion channels, and are thus useful for treating a disorder or disease of a living animal body, including a human, which disorder or disease is responsive to the partial or complete blockade of voltage gated ion channels of the central nervous system comprising the step of administering to such a living animal body, including a human, in need thereof a therapeutically effective amount of a pharmaceutical composition of the present invention.

Voltage-gated calcium channels are present in neurons, and in cardiac, smooth, and skeletal muscle and other excitable cells, and are known to play a variety of roles in membrane excitability, muscle contraction, and cellular secretion, such as in synaptic transmission (McCleskey). In neuronal cells, voltage-gated calcium channels have been classified by their electrophysiological as well as by their biochemical (binding) properties. Six classes of physiologically distinct calcium channels have been identified to date, namely the T, L, N, P, Q, and R-type channels.

It is well known that an accumulation of calcium (calcium overload) in the brain is seen after anoxia, ischemia, migraine and other hyperactivity periods of the brain, such as after epileptic convulsions. An uncontrolled high concentration of calcium in the cells of the central nervous system (CNS) is known to cause most of the degenerative changes connected with the above diseases. Compounds which can block the calcium channels of brain cells are therefore useful in the treatment of stroke, anoxia, ischemia, migraine, psychosis, or epilepsy, any other convulsive disorder and in the prevention of the degenerative changes connected with the same.

Compounds blocking the so called L-type calcium channels in the CNS are useful for the treatment of the above disorders by directly blocking the calcium uptake in the CNS.

Further, it is well known that the so called N- and P-types of calcium channels, as well as possibly other types of calcium channels, are involved in the regulation of neurotransmitter release. Compounds blocking the N- and/or P-types of calcium channels indirectly and very powerfully prevent calcium overload in the CNS after the hyperactivity periods of the brain as described above by inhibiting the enhanced neurotransmitter release seen after such hyperactivity periods of the CNS, and especially the neurotoxic, enhanced glutamate release after such hyperactivity periods of the CNS. Furthermore, blockers of the N- and/or P-types of calcium channels, as dependent upon the selectivity of the compound in question, inhibit the release of various other neurotransmitters such as aspartate, GABA, glycine, dopamine, serotonin and noradrenaline.

Thus, the pharmaceutical compositions of the present invention are useful as neuroprotectants, cardiovascular agents, anticonvulsants, analgesics or adjuvants to general anesthetics. A “neurological disorder or disease” is a disorder or disease of the nervous system including, but not limited to, global and focal ischemic and hemorrhagic stroke, head trauma, spinal cord injury, hypoxia-induced nerve cell damage as in cardiac arrest or neonatal distress or epilepsy. In addition, a “neurological disorder or disease” is a disease state and condition in which a neuroprotectant, anticonvulsant, analgesic and/or as an adjunct in general anesthesia may be indicated, useful, recommended or prescribed.

More specifically, the present invention is directed to the use of these compounds for the treatment and alleviation of epilepsy and as a general anticonvulsant agent. The present invention is also directed to the use of these compounds for reducing neurotoxic injury associated with conditions of hypoxia, anoxia or ischemia which typically follows stroke, cerebrovascular accident, brain or spinal cord trauma, myocardial infarct, physical trauma, drowning, suffocation, perinatal asphyxia, or hypoglycemic events. The present invention is further directed to the use of these compounds for treating pain, including acute and chronic pain, such migraine, nociceptive and neuropathic pain. Other uses of these compounds are described in U.S. Pat. No. 5,859,186, incorporated herein by reference.

A “neuroprotectant” is a compound capable of preventing the neuronal death associated with a neurological disorder or disease. An “anticonvulsant” is a compound capable of reducing convulsions produced by conditions such as simple partial seizures, complex partial seizures, status epilepticus, and trauma-induced seizures such as occur following head injury, including head surgery. An “analgesic” is a compound capable of relieving pain by altering perception of nociceptive stimuli without producing anesthesia or loss of consciousness. A “muscle relaxant” is a compound that reduces muscular tension. A “adjunct in general anesthesia” is a compound useful in conjunction with anesthetic agents in producing the loss of ability to perceive pain associated with the loss of consciousness.

The invention relates as well to methods useful for treatment of neurological disorders and diseases, including, but not limited to, global and focal ischemic and hemorrhagic stroke, head trauma, spinal cord injury, hypoxia-induced nerve cell damage such as in cardiac arrest or neonatal distress, epilepsy or other convulsive disorders without undesirable side effects.

Thus, in one embodiment, the invention provides a method of reducing/alleviating/ decreasing the perception of pain by a subject or for inducing analgesia in a subject comprising administering to the subject an effective amount of the pharmaceutical composition comprising a therapeutically effective amount of a ω-conopeptide described herein or a pharmaceutically acceptable salt or solvate thereof. The pain may be acute, persistent, inflammatory or neuropathic pain.

In a second embodiment, the invention provides a method of treating stroke, head or spinal cord trauma or injury, anoxia, hypoxia-induced nerve cell damage, ischemia, migraine, psychosis, anxiety, schizophrenia, inflammation, movement disorder, epilepsy, any other convulsive disorder or in the prevention of the degenerative changes connected with the same in a subject comprising administering to the subject an effective amount of the pharmaceutical composition comprising a therapeutically effective amount of a ω-conopeptide described herein or a pharmaceutically acceptable salt or solvate thereof.

The ω-conopeptides described herein are sufficiently small to be chemically synthesized. General chemical syntheses for preparing the foregoing ω-conotoxin peptides are described hereinafter. Various ones of the ω-conopeptides can also be obtained by isolation and purification from specific Conus species using the technique described in U.S. Pat. Nos. 4,447,356 (Olivera et al., 1984); 5,514,774; 5,719,264; and 5,591,821, as well as in PCT published application WO 98/03189, the disclosures of which are incorporated herein by reference.

Although the ω-conopeptides of the present invention can be obtained by purification from cone snails, because the amounts of ω-conopeptides obtainable from individual snails are very small, the desired substantially pure co-conopeptides are best practically obtained in commercially valuable amounts by chemical synthesis using solid-phase strategy. For example, the yield from a single cone snail may be about 10 micrograms or less of ω-conopeptides peptide. By “substantially pure” is meant that the peptide is present in the substantial absence of other biological molecules of the same type; it is preferably present in an amount of at least about 85% purity and preferably at least about 95% purity. Chemical synthesis of biologically active ω-conopeptides peptides depends of course upon correct determination of the amino acid sequence.

The ω-conopeptides can also be produced by recombinant DNA techniques well known in the art. Such techniques are described by Sambrook et al. (1989). A gene of interest (i.e., a gene that encodes a suitable ω-conopeptides) can be inserted into a cloning site of a suitable expression vector by using standard techniques. These techniques are well known to those skilled in the art. The expression vector containing the gene of interest may then be used to transfect the desired cell line. Standard transfection techniques such as calcium phosphate co-precipitation, DEAE-dextran transfection or electroporation may be utilized. A wide variety of host/expression vector combinations may be used to express a gene encoding a conotoxin peptide of interest. Such combinations are well known to a skilled artisan. The peptides produced in this manner are isolated, reduced if necessary, and oxidized to form the correct disulfide bonds.

One method of forming disulfide bonds in the ω-conopeptides of the present invention is the air oxidation of the linear peptides for prolonged periods under cold room temperatures or at room temperature. This procedure results in the creation of a substantial amount of the bioactive, disulfide-linked peptides. The oxidized peptides are fractionated using reverse-phase high performance liquid chromatography (HPLC) or the like, to separate peptides having different linked configurations. Thereafter, either by comparing these fractions with the elution of the native material or by using a simple assay, the particular fraction having the correct linkage for maximum biological potency is easily determined. However, because of the dilution resulting from the presence of other fractions of less biopotency, a somewhat higher dosage may be required.

The peptides are synthesized by a suitable method, such as by exclusively solid-phase techniques, by partial solid-phase techniques, by fragment condensation or by classical solution couplings.

In conventional solution phase peptide synthesis, the peptide chain can be prepared by a series of coupling reactions in which constituent amino acids are added to the growing peptide chain in the desired sequence. Use of various coupling reagents, e.g., dicyclohexylcarbodiimide or diisopropylcarbonyldimidazole, various active esters, e.g., esters of N-hydroxyphthalimide or N-hydroxy-succinimide, and the various cleavage reagents, to carry out reaction in solution, with subsequent isolation and purification of intermediates, is well known classical peptide methodology. Classical solution synthesis is described in detail in the treatise, “Methoden der Organischen Chemie (Houben-Weyl): Synthese von Peptiden,” (1974).

Techniques of exclusively solid-phase synthesis are set forth in the textbook, “Solid-Phase Peptide Synthesis,” (Stewart and Young, 1969), and are exemplified by the disclosure of U.S. Pat. No. 4,105,603 (Vale et al., 1978). The fragment condensation method of synthesis is exemplified in U.S. Pat. No. 3,972,859 (1976). Other available syntheses are exemplified by U.S. Pat. Nos. 3,842,067 (1974) and 3,862,925 (1975). The synthesis of peptides containing γ-carboxyglutamic acid residues is exemplified by Rivier et al. (1987), Nishiuchi et al. (1993) and Zhou et al. (1996).

Common to such chemical syntheses is the protection of the labile side chain groups of the various amino acid moieties with suitable protecting groups which will prevent a chemical reaction from occurring at that site until the group is ultimately removed. Usually also common is the protection of an α-amino group on an amino acid or a fragment while that entity reacts at the carboxyl group, followed by the selective removal of the α-amino protecting group to allow subsequent reaction to take place at that location. Accordingly, it is common that, as a step in such a synthesis, an intermediate compound is produced which includes each of the amino acid residues located in its desired sequence in the peptide chain with appropriate side-chain protecting groups linked to various ones of the residues having labile side chains.

As far as the selection of a side chain amino protecting group is concerned, generally one is chosen which is not removed during deprotection of the α-amino groups during the synthesis. However, for some amino acids, e.g., His, protection is not generally necessary. In selecting a particular side chain protecting group to be used in the synthesis of the peptides, the following general rules are followed: (a) the protecting group preferably retains its protecting properties and is not split off under coupling conditions, (b) the protecting group should be stable under the reaction conditions selected for removing the α-amino protecting group at each step of the synthesis, and (c) the side chain protecting group must be removable, upon the completion of the synthesis containing the desired amino acid sequence, under reaction conditions that will not undesirably alter the peptide chain.

It should be possible to prepare many, or even all, of these peptides using recombinant DNA technology. However, when peptides are not so prepared, they are preferably prepared using the Merrifield solid-phase synthesis, although other equivalent chemical syntheses known in the art can also be used as previously mentioned. Solid-phase synthesis is commenced from the C-terminus of the peptide by coupling a protected α-amino acid to a suitable resin. Such a starting material can be prepared by attaching an α-amino-protected amino acid by an ester linkage to a chloromethylated resin or a hydroxymethyl resin, or by an amide bond to a benzhydrylamine (BHA) resin or paramethylbenzhydrylamine (MBHA) resin. Preparation of the hydroxymethyl resin is described by Bodansky et al. (1966). Chloromethylated resins are commercially available from Bio Rad Laboratories (Richmond, Calif.) and from Lab. Systems, Inc. The preparation of such a resin is described by Stewart and Young (1969). BHA and MBHA resin supports are commercially available, and are generally used when the desired polypeptide being synthesized has an unsubstituted amide at the C-terminus. Thus, solid resin supports may be any of those known in the art, such as one having the formulae —O—CH₂-resin support, —NH BHA resin support, or —NH—MBHA resin support. When the unsubstituted amide is desired, use of a BHA or MBHA resin is preferred, because cleavage directly gives the amide. In case the N-methyl amide is desired, it can be generated from an N-methyl BHA resin. Should other substituted amides be desired, the teaching of U.S. Pat. No. 4,569,967 (Kornreich et al., 1986) can be used, or should still other groups than the free acid be desired at the C-terminus, it may be preferable to synthesize the peptide using classical methods as set forth in the Houben-Weyl text (1974).

The C-terminal amino acid, protected by Boc or Fmoc and by a side-chain protecting group, if appropriate, can be first coupled to a chloromethylated resin according to the procedure set forth in K. Horiki et al. (1978), using KF in DMF at about 60° C. for 24 hours with stirring, when a peptide having free acid at the C-terminus is to be synthesized. Following the coupling of the BOC-protected amino acid to the resin support, the α-amino protecting group is removed, as by using trifluoroacetic acid (TFA) in methylene chloride or TFA alone. The deprotection is carried out at a temperature between about 0° C. and room temperature. Other standard cleaving reagents, such as HCl in dioxane, and conditions for removal of specific α-amino protecting groups may be used as described in Schroder & Lubke (1965).

After removal of the α-amino-protecting group, the remaining α-amino- and side chain-protected amino acids are coupled step-wise in the desired order to obtain the intermediate compound defined hereinbefore, or as an alternative to adding each amino acid separately in the synthesis, some of them may be coupled to one another prior to addition to the solid phase reactor. Selection of an appropriate coupling reagent is within the skill of the art. Particularly suitable as a coupling reagent is N,N′-dicyclohexylcarbodiimide (DCC, DIC, HBTU, HATU, TBTU in the presence of HoBt or HoAt).

The activating reagents used in the solid phase synthesis of the peptides are well known in the peptide art. Examples of suitable activating reagents are carbodiimides, such as N,N′-diisopropylcarbodiimide and N-ethyl-N′-(3-dimethylaminopropyl)carbodiimide. Other activating reagents and their use in peptide coupling are described by Schroder & Lubke (1965) and Kapoor (1970).

Each protected amino acid or amino acid sequence is introduced into the solid-phase reactor in about a twofold or more excess, and the coupling may be carried out in a medium of dimethylformamide (DMF):CH₂Cl₂ (1:1) or in DMF or CH₂Cl₂ alone. In cases where intermediate coupling occurs, the coupling procedure is repeated before removal of the α-amino protecting group prior to the coupling of the next amino acid. The success of the coupling reaction at each stage of the synthesis, if performed manually, is preferably monitored by the ninhydrin reaction, as described by Kaiser et al. (1970). Coupling reactions can be performed automatically, as on a Beckman 990 automatic synthesizer, using a program such as that reported in Rivier et al. (1978).

After the desired amino acid sequence has been completed, the intermediate peptide can be removed from the resin support by treatment with a reagent, such as liquid hydrogen fluoride or TFA (if using Fmoc chemistry), which not only cleaves the peptide from the resin but also cleaves all remaining side chain protecting groups and also the -amino protecting group at the N-terminus if it was not previously removed to obtain the peptide in the form of the free acid. If Met is present in the sequence, the Boc protecting group is preferably first removed using trifluoroacetic acid (TFA)/ethanedithiol prior to cleaving the peptide from the resin with HF to eliminate potential S-alkylation. When using hydrogen fluoride or TFA for cleaving, one or more scavengers such as anisole, cresol, dimethyl sulfide and methylethyl sulfide are included in the reaction vessel.

Cyclization of the linear peptide is preferably affected, as opposed to cyclizing the peptide while a part of the peptido-resin, to create bonds between Cys residues. To effect such a disulfide cyclizing linkage, fully protected peptide can be cleaved from a hydroxymethylated resin or a chloromethylated resin support by ammonolysis, as is well known in the art, to yield the fully protected amide intermediate, which is thereafter suitably cyclized and deprotected. Alternatively, deprotection, as well as cleavage of the peptide from the above resins or a benzhydrylamine (BHA) resin or a methylbenzhydrylamine (MBHA), can take place at 0° C. with hydrofluoric acid (HF) or TFA, followed by oxidation as described above.

The peptides are also synthesized using an automatic synthesizer. Amino acids are sequentially coupled to an MBHA Rink resin (typically 100 mg of resin) beginning at the C-terminus using an Advanced Chemtech 357 Automatic Peptide Synthesizer. Couplings are carried out using 1,3-diisopropylcarbodimide in N-methylpyrrolidinone (NMP) or by 2-(1H-benzotriazole-1-yl)-1,1,3,3-tetramethyluronium hexafluorophosphate (HBTU) and diethylisopropylethylamine (DIEA). The FMOC protecting group is removed by treatment with a 20% solution of piperidine in dimethylformamide(DMF). Resins are subsequently washed with DMF (twice), followed by methanol and NMP.

Muteins, analogs or active fragments, of the foregoing conotoxin peptides are also contemplated here. See, e.g., Hammerland et al. (1992). Derivative muteins, analogs or active fragments of the conotoxin peptides may be synthesized according to known techniques, including conservative amino acid substitutions, such as outlined in U.S. Pat. Nos. 5,545,723 (see particularly col. 2, line 50—col. 3, line 8); 5,534,615 (see particularly col. 19, line 45—col 22, line 33); and 5,364,769 (see particularly col. 4, line 55—col. 7, line 26), each herein incorporated by reference.

The ω-conopeptides of the present invention are also useful to reduce neurotoxic injury associated with conditions of hypoxia, anoxia or ischemia which typically follows stroke, cerebrovascular accident, brain or spinal chord trauma, myocardial infarct, physical trauma, drownings, suffocation, perinatal asphyxia, or hypoglycemic events. To reduce neurotoxic injury, an ω-conopeptide should be administered in a therapeutically effective amount to the patient within 24 hours of the onset of the hypoxic, anoxic or ischemic condition in order for the ω-conopeptide to effectively minimize the CNS damage which the patient will experience.

The ω-conopeptides of the present invention are further useful in controlling pain, e.g., as analgesic agents, and the treatment of migraine, acute pain or persistent pain. They can be used prophylactically or to relieve the symptoms associated with a migraine episode, or to treat acute or persistent pain. For these uses, an ω-conopeptide is administered in a therapeutically effective amount to overcome or to ease the pain.

Pharmaceutical compositions containing a compound of the present invention as the active ingredient can be prepared according to conventional pharmaceutical compounding techniques. See, for example, Remington's Pharmaceutical Sciences, 18th Ed. (1990, Mack Publishing Co., Easton, Pa.). Typically, an antagonistic amount of active ingredient will be admixed with a pharmaceutically acceptable carrier. The carrier may take a wide variety of forms depending on the form of preparation desired for administration, e.g., intravenous, oral, parenteral or intrathecally. For examples of delivery methods see U.S. Pat. No. 5,844,077, incorporated herein by reference.

“Pharmaceutical composition” means physically discrete coherent portions suitable for medical administration. “Pharmaceutical composition in dosage unit form” means physically discrete coherent units suitable for medical administration, each containing a daily dose or a multiple (up to four times) or a sub-multiple (down to a fortieth) of a daily dose of the active compound in association with a carrier and/or enclosed within an envelope. Whether the composition contains a daily dose, or for example, a half, a third or a quarter of a daily dose, will depend on whether the pharmaceutical composition is to be administered once or, for example, twice, three times or four times a day, respectively.

The term “salt”, as used herein, denotes acidic and/or basic salts, formed with inorganic or organic acids and/or bases, preferably basic salts. While pharmaceutically acceptable salts are preferred, particularly when employing the compounds of the invention as medicaments, other salts find utility, for example, in processing these compounds, or where non-medicament-type uses are contemplated. Salts of these compounds may be prepared by art-recognized techniques.

Examples of such pharmaceutically acceptable salts include, but are not limited to, inorganic and organic addition salts, such as hydrochloride, sulphates, nitrates or phosphates and acetates, trifluoroacetates, propionates, succinates, benzoates, citrates, tartrates, fumarates, maleates, methane-sulfonates, isothionates, theophylline acetates, salicylates, respectively, or the like. Lower alkyl quaternary ammonium salts and the like are suitable, as well.

As used herein, the term “pharmaceutically acceptable” carrier means a non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations.

Wetting agents, emulsifiers and lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Examples of pharmaceutically acceptable antioxidants include, but are not limited to, water soluble antioxidants such as ascorbic acid, cysteine hydrochloride, sodium bisulfite, sodium metabisulfite, sodium sulfite, and the like; oil soluble antioxidants, such as ascorbyl palmitate, butylated hydroxyanisole (BHA), butylated hydroxytoluene (BHT), lecithin, propyl gallate, aloha-tocopherol and the like; and the metal chelating agents such as citric acid, ethylenediamine tetraacetic acid (EDTA), sorbitol, tartaric acid, phosphoric acid and the like.

For oral administration, the compounds can be formulated into solid or liquid preparations such as capsules, pills, tablets, lozenges, melts, powders, suspensions or emulsions.

In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier. See for example, WO 96/11698.

For parenteral administration, the compound may be dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

A variety of administration routes are available. The particular mode selected will depend of course, upon the particular drug selected, the severity of the disease state being treated and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, sublingual, topical, nasal, transdermal or parenteral routes. The term “parenteral” includes subcutaneous, intravenous, epidural, irrigation, intramuscular, release pumps, or infusion.

For example, administration of the active agent according to this invention may be achieved using any suitable delivery means, including:

(a) pump (see, e.g., Luer & Hatton (1993), Zimm et al. (1984) and Ettinger et al. (1978));

(b), microencapsulation (see, e.g., U.S. Pat. Nos. 4,352,883; 4,353,888; and 5,084,350);

(c) continuous release polymer implants (see, e.g., U.S. Pat. No. 4,883,666);

(d) macroencapsulation (see, e.g., U.S. Pat. Nos. 5,284,761, 5,158,881, 4,976,859 and 4,968,733 and published PCT patent applications WO92/19195, WO 95/05452);

(e) naked or unencapsulated cell grafts to the CNS (see, e.g., U.S. Pat. Nos. 5,082,670 and 5,618,531);

(f) injection, either subcutaneously, intravenously, intra-arterially, intramuscularly, or to other suitable site; or

(g) oral administration, in capsule, liquid, tablet, pill, or prolonged release formulation.

In one embodiment of this invention, an active agent is delivered directly into the CNS, preferably to the brain ventricles, brain parenchyma, the intrathecal space or other suitable CNS location, most preferably intrathecally.

Alternatively, targeting therapies may be used to deliver the active agent more specifically to certain types of cell, by the use of targeting systems such as antibodies or cell specific ligands. Targeting may be desirable for a variety of reasons, e.g. if the agent is unacceptably toxic, or if it would otherwise require too high a dosage, or if it would not otherwise be able to enter the target cells.

The active agents, which are peptides, can also be administered in a cell based delivery system in which a DNA sequence encoding an active agent is introduced into cells designed for implantation in the body of the patient, especially in the spinal cord region.

Suitable delivery systems are described in U.S. Pat. No. 5,550,050 and published PCT Application Nos. WO 92/19195, WO 94/25503, WO 95/01203, WO 95/05452, WO 96/02286, WO 96/02646, WO 96/40871, WO 96/40959 and WO 97/12635. Suitable DNA sequences can be prepared synthetically for each active agent on the basis of the developed sequences and the known genetic code.

The active agent is preferably administered in an therapeutically effective amount. By a “therapeutically effective amount” or simply “effective amount” of an active compound is meant a sufficient amount of the compound to treat the desired condition at a reasonable benefit/risk ratio applicable to any medical treatment. The actual amount administered, and the rate and time-course of administration, will depend on the nature and severity of the condition being treated. Prescription of treatment, e.g. decisions on dosage, timing, etc., is within the responsibility of general practitioners or spealists, and typically takes account of the disorder to be treated, the condition of the individual patient, the site of delivery, the method of administration and other factors known to practitioners. Examples of techniques and protocols can be found in Remington 's Parmaceutical Sciences.

Dosage may be adjusted appropriately to achieve desired drug levels, locally or systemically. Typically the active agents of the present invention exhibit their effect at a dosage range from about 0.001 mg/kg to about 250 mg/kg, preferably from about 0.01 mg/kg to about 100 mg/kg of the active ingredient, more preferably from a bout 0.05 mg/kg to about 75 mg/kg. A suitable dose can be administered in multiple sub-doses per day. Typically, a dose or sub-dose may contain from about 0.1 mg to about 500 mg of the active ingredient per unit dosage form. A more preferred dosage will contain from about 0.5 mg to about 100 mg of active ingredient per unit dosage form. Dosages are generally initiated at lower levels and increased until desired effects are achieved. In the event that the response in a subject is insufficient at such doses, even higher doses (or effective higher doses by a different, more localized delivery route) may be employed to the extent that patient tolerance permits. Continuous dosing over, for example 24 hours or multiple doses per day are contemplated to achieve appropriate systemic levels of compounds.

For the treatment of pain, if the route of administration is directly to the CNS, the dosage contemplated is from about 1 ng to about 100 mg per day, preferably from about 100 ng to about 10 mg per day, more preferably from about 1 μg to about 100 μg per day. If administered peripherally, the dosage contemplated is somewhat higher, from about 100 ng to about 1000 mg per day, preferably from about 10 μg to about 100 mg per day, more preferably from about 100 μg to about 10 mg per day. If the conopeptide is delivered by continuous infusion (e.g., by pump delivery, biodegradable polymer delivery or cell-based delivery), then a lower dosage is contemplated than for bolus delivery.

Advantageously, the compositions are formulated as dosage units, each unit being adapted to supply a fixed dose of active ingredients. Tablets, coated tablets, capsules, ampoules and suppositories are examples of dosage forms according to the invention.

It is only necessary that the active ingredient constitute an effective amount, i.e., such that a suitable effective dosage will be consistent with the dosage form employed in single or multiple unit doses. The exact individual dosages, as well as daily dosages, are determined according to standard medical principles under the direction of a physician or veterinarian for use humans or animals.

The pharmaceutical compositions will generally contain from about 0.0001 to 99 wt. %, preferably about 0.001 to 50 wt. %, more preferably about 0.01 to 10 wt. % of the active ingredient by weight of the total composition. In addition to the active agent, the pharmaceutical compositions and medicaments can also contain other pharmaceutically active compounds. Examples of other pharmaceutically active compounds include, but are not limited to, analgesic agents, cytokines and therapeutic agents in all of the major areas of clinical medicine. When used with other pharmaceutically active compounds, the conopeptides of the present invention may be delivered in the form of drug cocktails. A cocktail is a mixture of any one of the compounds useful with this invention with another drug or agent. In this embodiment, a common administration vehicle (e.g., pill, tablet, implant, pump, injectable solution, etc.) would contain both the instant composition in combination supplementary potentiating agent. The individual drugs of the cocktail are each administered in therapeutically effective amounts. A therapeutically effective amount will be determined by the parameters described above; but, in any event, is that amount which establishes a level of the drugs in the area of body where the drugs are required for a period of time which is effective in attaining the desired effects.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982; Sambrook et al., 1989; Ausubel et al., 1992;

Glover, 1985; Anand, 1992; Guthrie and Fink, 1991; Harlow and Lane, 1988; Jakoby and Pastan, 1979; Nucleic Acid Hybridization (B. D. Hames & S. J. Higgins eds. 1984);

Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986).

### EXAMPLES

The present invention is described by reference to the following Examples, which are offered by way of illustration and are not intended to limit the invention in any manner.

Standard techniques well known in the art or the techniques specifically described below were utilized.

### Example 1

### Isolation of ω-Conotoxins

Crude venom was extracted from venom ducts (Cruz et al., 1976), and the components were purified as previously described (Cartier et al., 1996). The crude extract from venom ducts was purified by reverse phase liquid chromatography (RPLC) using a Vydac C₁₈ semi-preparative column (10×250 mm). Further purification of bioactive peaks was done on a Vydac C₁₈ analytical column (4.6×220 mm). The effluents were monitored at 220 nm. Peaks were collected, and aliquots were assayed for activity. Throughout purification, HPLC fractions were assayed by means of intracerebral ventricular (i.c.v.) injection into mice (Clark et al., 1981).

The amino acid sequence of the purified peptides were determined by standard methods. The purified peptides were reduced and alkylated prior to sequencing by automated Edman degradation on an Applied Biosystems 477A Protein Sequencer with a 120A Analyzer (DNA/Peptide Facility, University of Utah) (Martinez et al., 1995; Shon et al., 1994).

In accordance with this method, the ω-conopeptides described as “isolated” in Table 1 were obtained. These ω-conopeptides, as well as the other ω-conopeptides and the ω-conopeptide precursors set forth in Table 1 are synthesized as described in U.S. Pat. No. 5,591,821.

### Example 2

Isolation of DNA Encoding ω-Conopeotides

DNA coding for ω-conopeptides was isolated and cloned in accordance with conventional techniques using general procedures well known in the art, such as described in Olivera et al. (1996). Alternatively, cDNA libraries was prepared from Conus venom duct using conventional techniques. DNA from single clones was amplified by conventional techniques using primers which correspond approximately to the M13 universal priming site and the M13 reverse universal priming site. Clones having a size of approximately 300-500 nucleotides were sequenced and screened for similarity in sequence to known ω-conotoxins. The DNA sequences and encoded propeptide sequences are set forth in Table 1. DNA sequences coding for the mature toxin can also be prepared on the basis of the DNA sequences set forth in Table1. An alignment of the ω-conopeptides of the present invention is set forth in Table 2.

TABLE 1
DNA and Amino Acid Sequences of ω-Conopeptides and Precursors

| | |
|---|---|
| Name: J410 | |
| Species: | |
| Cloned: Yes | |
| DNA Sequence: | |
| GGATCCATGAAACTGACGTGCATGGTGATCGTCGCCGTGCTGCTCCTGACGGCCTGT | (SEQ ID NO:1) |
| CAACTCATCACAGCTGATGACTCCAGAGGTACGCAGAAGCATCATGCCCTGAGGTC | |
| GACCACCAATTTCTCCACGTTGACTCGTCGCTGCCTTTCTCCCGGATCACGATGTCA | |
| TAAGACAATGCGTAACTGCTGCACTTCATGCTCTTCATACAAAGGGAAATGTCGGCC | |
| TCGAAAATGAACCACTCATCACCTACTCCTCTGGAGGCCTCAGAGGAATTACATTGA | |
| AATAAAAGCCGCATTACAAAAAAAAAAAAAAAAA | |
| Translation: | |
| MKLTCMVIVAVLLLTACQLITADDSRGTQKHHALRSTTNFSTLTRRCLSPGSRCHKTMR | (SEQ ID NO:2) |
| NCCTSCSSYKGKCRPRK | |
| Toxin Sequence: | |
| Cys-Leu-Ser-Xaa3-Gly-Ser-Arg-Cys-His-Lys-Thr-Met-Arg-Asn-Cys-Cys-Thr-Ser-Cys-Ser-Ser- | (SEQ ID NO:3) |
| Xaa5-Lys-Gly-Lys-Cys-Arg-Xaa3-Arg-Lys-\hat{ } | |
| Name: J411 | |
| Species: | |
| Cloned: Yes | |
| DNA Sequence: | |
| GGATCCATGAAACTGACGTGCGTGGTGATCGTCGCCGTGCTGCTCCTGACGGTCTGT | (SEQ ID NO:4) |
| CAACTCATCACAGCTGATGACTCCAGAGGTACGCAGAAGCATCATGCCCTGAGGTC | |
| GACCACCAATTTCTCCACGTCGACTCGTCGCTGCAAACCTCCCGGAAGAAAATGTCT | |
| GAATAGAAAGAATGAATGCTGCAGCAAGTTTTGCAATGAACACCTACATATGTGTG | |
| GATAAATGGCTAAAAACTGAATAAAAGCCGCATTGCAAAAAAAAAAAAAAAAAAA | |
| AA | |
| Translation: | |
| MKLTCVVIVAVLLLTVCQLITADDSRGTQKHHALRSTTNFSTSTRRCKPPGRKCLNRKN | (SEQ ID NO:5) |
| ECCSKFCNEHLHMCG | |
| Toxin Sequence: | |
| Cys-Lys-Xaa3-Xaa3-Gly-Arg-Lys-Cys-Leu-Asn-Arg-Lys-Asn-Xaa1-Cys-Cys-Ser-Lys-Phe- | (SEQ ID NO:6) |
| Cys-Asn-Xaa1-His-Leu-His-Met-Cys-# | |
| Name: J413 | |
| Species: | |
| Cloned: Yes | |
| DNA Sequence: | |
| GGATCCATGAAACTGACGTGCGTGGTGATCGTCGCCGTGCTGCTCCTGACGGCCTGT | (SEQ ID NO:7) |
| CAACTCGTCACAGCTGATGGCTCCAGAGGTATGCAGAAGCATTATGCCCTGAGGTC | |
| GACCACCAATCTCTCCATATCGTCTCGCTGCAAACCTCCCAGAAGAAAATGTCTGAA | |
| GATTAAGGATAAATGCTGCAACTTTTGCAATACACACCTAAATATGTGTGGATAAAT | |
| GGCTAAAAACTGAATAAAAGCCGCATTGCAAAAAAAAAAAAAAAAAAA | |
| Translation: | |
| MKLTCVVIVAVLLLTACQLVTADGSRGMQKHYALRSTTNLSISSRCKPPRRKCLKIKDK | (SEQ ID NO:8) |
| CCNFCNTHLNMCG | |
| Toxin Sequence: | |
| Cys-Lys-Xaa3-Xaa3-Arg-Arg-Lys-Cys-Leu-Lys-Ile-Lys-Asp-Lys-Cys-Cys-Asn-Phe-Cys-Asn- | (SEQ ID NO:9) |
| Thr-His-Leu-Asn-Met-Cys-# | |
| Name: J414 | |
| Species: | |
| Cloned: Yes | |
| DNA Sequence: | |
| GGATCCATGAAACTGACGTGTGTGGTGATCGTCGCCGTGCTGCTCCTGATGGCCTGT | (SEQ ID NO:10) |
| CAACTCGTCACAGCTGATGGCTCCAGAGGTATGCACAAGCATTATGCCCTGAGGTC | |
| GACCACCAAACTCTCCATGTCGACTCGCTGCGCAGGTCCAGGAACAATTTGTCCTAA | |
| TAGGGTATGCTGCGGTTATTGCAGTAAAAGAACACATCTATGTCATTCGCGAACTGG | |
| CTGATCTTCCCCCTTCTGCGCTCCATCCTTTTCTGCCTGAGTCCTCCATACCTGAGAA | |
| TGGTCATGAACCACTCAACACCTACTCCTCTGGAGGGCCTCAGAAGAGCTACATTG | |
| AAATAAAAGCCGCATTACAAAAAAAAAAAAAAAAAA | |
| Translation: | |
| MKLTCVVIVAVLLLMACQLVTADGSRGMHKHYALRSTTKLSMSTRCAGPGTICPNRVC | (SEQ ID NO:11) |
| CGYCSKRTHLCHSRTG | |
| Toxin Sequence: | |
| Cys-Ala-Gly-Xaa3-Gly-Thr-Ile-Cys-Xaa3-Asn-Arg-Val-Cys-Cys-Gly-Xaa5-Cys-Ser-Lys-Arg- | (SEQ ID NO:12) |
| Thr-His-Leu-Cys-His-Ser-Arg-Thr-# | |
| Name: Ar6.10 | |
| Species: arenatus | |
| Cloned: Yes | |
| DNA Sequence: | |
| GGATCCATGAAACTGACGTGCATGGTGATCATCGCCGTGCTGTTCCTGACGGCCTGT | (SEQ ID NO:13) |
| CAACTCATTACAGGTGAGCAGAAGGACCATGCTCTGAGGTCAACTGACAAAAACTC | |
| CAAGTTGACTAGGCAGTGCTCGGCTAACGGTGGATCTTGTACTCGTCATTTTCACTG | |
| CTGCAGCCTCTATTGCAATAAAGATTCCAGTGTATGTGTGGCAACCTCATACCCGTG | |
| AGTGGCCATGAACCCCTCAATACCCTCTCCTCTGGAGGCTTCAGAGGAACTGCATTG | |
| AAATAAAACCGCATTGCAATAAAAAAAAAAAAAAAAAAA | |
| Translation: | |
| MKLTCMVIIAVLFLTACQLITGEQKDHALRSTDKNSKLTRQCSANGGSCTRHFHCCSLY | (SEQ ID NO:14) |
| CNKDSSVCVATSYP | |
| Toxin Sequence: | |
| Xaa2-Cys-Ser-Ala-Asn-Gly-Gly-Ser-Cys-Thr-Arg-His-Phe-His-Cys-Cys-Ser-Leu-Xaa5-Cys- | (SEQ ID NO:15) |
| Asn-Lys-Asp-Ser-Ser-Val-Cys-Val-Ala-Thr-Ser-Xaa5-Xaa3-\hat{ } | |
| Name: Ar6.2 | |
| Species: arenatus | |
| Cloned: Yes | |
| DNA Sequence: | |
| ACCAAAACCATCATCAAAATGAAACTGACGTGCGTGTTGATTATCGCCGTGCTGTTC | (SEQ ID NO:16) |
| CTGACGGCCTGTCAACTCATTACAGCTGAGACTTACTCCAGAGGTGAGCAGAAGCA | |
| CCATGCTCTGAGGTCAACTGACAGAAACTCCAAGTTGACCAGGACATGCAACACTC | |
| CCACTGAATATTGTACTTTGCATCGACACTGCTGCAGCGGCTACTGCCATAAAACAA | |
| TCCAGGCATGTTCATAATACCGGTGAGTGGTCATGAACCACTCAATACCCTCTCCTC | |
| TGGAGGCTTCAGAGGAACTGCATTGAAATAAAAGCCGCATTGC | |
| Translation: | |
| MKLTCVLIIAVLFLTACQLITAETYSRGEQKHHALRSTDRNSKLTRTCNTPTEYCTLHRH | (SEQ ID NO:17) |
| CCSGYCHKTIQACS | |
| Toxin Sequence: | |
| Thr-Cys-Asn-Thr-Xaa3-Thr-Xaa1-Xaa5-Cys-Thr-Leu-His-Arg-His-Cys-Cys-Ser-Gly-Xaa5- | (SEQ ID NO:18) |
| Cys-His-Lys-Thr-Ile-Gln-Ala-Cys-Ser-\hat{ } | |
| Name: Ar6.3 | |
| Species: arenatus | |
| Cloned: Yes | |
| DNA Sequence: | |
| ACCAAAACCATCATCAAAATGAAACTGACGTGCGTGTTGATCATCGCCGTGCTGTTC | (SEQ ID NO:19) |
| CTGACGGCCTGTCAACTCATTACAGCTGAGACTTACTCCAGAGGTGAGCAGATGCA | |
| CCGTGCTCTGAGGTCAACTGACAAAAACTCCAAGTTGACTAGGCAGTGCACGCCTA | |
| ACGGTGGATCTTGTTCTCGTCATTTTCACTGCTGCAGCCTCTATTGCAATAAAAGTA | |
| CTGGCGTATGTATTGCAACCTCATACCCGTGAGTGGTCATGAACCACTCAATACCCT | |
| CTCCTCTGGAGGCTTCAGAGGAACTGCATTGAAATAAAAGCCGCATTGC | |
| Translation: | |
| MRLTCVLIIAVLFLTACQLITAETYSRGEQMHRALRSTDKNSKLTRQCTPNGGSCSRHF | (SEQ ID NO:20) |
| HCCSLYCNKSTGVCIATSYP | |
| Toxin Sequence: | |
| Xaa2-Cys-Thr-Xaa3-Asn-Gly-Gly-Ser-Cys-Ser-Arg-His-Phe-His-Cys-Cys-Ser-Leu-Xaa5-Cys- | (SEQ ID NO:21) |
| Asn-Lys-Ser-Thr-Gly-Val-Cys-Ile-Ala-Thr-Ser-Xaa5-Xaa3-\hat{ } | |
| Name: Ar6.4 | |
| Species: arenatus | |
| Cloned: Yes | |
| DNA Sequence: | |
| GGATCCATGAAACTGACGTGCATGGTGATTATCGCCGTGCTGTTCCTGACGGCCTGT | (SEQ ID NO:22) |
| CAACTCATTACAGCTGAGACTTACTCCAGAGGTGAGCAGAAGCACCATGCTCTGAG | |
| GTCAACTGACAAAAACTCCAAGTTGACCAGGACATGCAACACTCCCACCGAATATT | |
| GTACTTTGCATCAACACTGCTGCAGCGGCTACTGCCATAAAACAATCCAGGCATGTT | |
| CATAATACCGGTGAGTGGTCATGAACCACTCAATACCCTCTCCTCTGGAGGCTTCAG | |
| AGGAACTGCATTGAAATAAAACCGCATTACAAAAAAAAAAAAAAAAAAA | |
| Translation: | |
| MKLTCMVIIAVLFLTACQLITAETYSRGEQKHHALRSTDKNSKLTRTCNTPTEYCTLHQ | (SEQ ID NO:23) |
| HCCSGYCHKTIQACS | |
| Toxin Sequence: | |
| Thr-Cys-Asn-Thr-Xaa3-Thr-Xaa1-Xaa5-Cys-Thr-Leu-His-Gln-His-Cys-Cys-Ser-Gly-Xaa5- | (SEQ ID NO:24) |
| Cys-His-Lys-Thr-Ile-Gln-Ala-Cys-Ser- | |
| Name: Ar6.6 | |
| Species: arenatus | |
| Cloned: Yes | |
| DNA Sequence: | |
| GGATCCATGAAACTGACGTGTATGGTGATCATCGCCGTACTGTTCCTGACGGCCTGT | (SEQ ID NO:25) |
| CAACTCATTACAGCTGAGACTTACTCCAGAGGTAAGCAGATGCACCGCGCTCTGAG | |
| GTCAACTGACAAAAACTCCCAGTTGACCAGGGAATGCACACCTCCCGGTGGAGCTT | |
| GTGGTTTACCTACACACTGCTGCGGGTTTTGCGATACTGCAAACAACAGATGTCTGT | |
| AAAGCTGGTCTGGCGTCTGATATTCCCCTTCTGTGCTCTATCCTCTTTGGCCTGAGTC | |
| ATCCGTACCTGTGAGTGGTCATGAACTACTCAATACCCTCTCCTCTGGAGGCTTCAG | |
| AGGAACTACAATGAAATAAAACCCGCATTGCAGAGAAAAAAAAAAAAAAAAAA | |
| Translation: | |
| MKLTCMVIIAVLFLTACQLITAETYSRGKQMHRALRSTDKNSQLTRECTPPGGACGLPT | (SEQ ID NO:26) |
| HCCGFCDTANNRCL | |
| Toxin Sequence: | |
| Xaa1-Cys-Thr-Xaa3-Xaa3-Gly-Gly-Ala-Cys-Gly-Leu-Xaa3-Thr-His-Cys-Cys-Gly-Phe-Cys- | (SEQ ID NO:27) |
| Asp-Thr-Ala-Asn-Asn-Arg-Cys-Leu-\hat{ } | |
| Name: Ar6.7 | |
| Species: arenatus | |
| Cloned: Yes | |
| DNA Sequence: | |
| GGATCCATGAAACTGACGTGCGTGGTGATTATCGCCGTGCTGTTCCTGACGGCCTGT | (SEQ ID NO:28) |
| CAACTCATTACAGCTGAGACTTACTCCAGAGGTGAGCAGAATCACCATGTTCTGAG | |
| GTCAACTGACAAAAACTCCAAGTTGACCAGGACATGCAACACTCCCACTGAATATT | |
| GTACTTTGCATCAACACTGCTGCAGCGGCCACTGCCATAAAACAATCCAGGCATGT | |
| GCATAATACCGGTGGGTGGTCATGAACCACTCAATACCCTCTCCTCTGGAGGCTTCA | |
| GAGGAACTGCATTGAAATAAAACCGCATTGCAATGAANAAAAAAAAAAAAAAAAA | |
| AAAAAAAA | |
| Translation: | |
| MKLTCVVIIAVLFLTACQLITAETYSRGEQNHHVLRSTDKNSKLTRTCNTPTEYCTLHQ | (SEQ ID NO:29) |
| HCCSGHCHKTIQACA | |
| Toxin Sequence: | |
| Thr-Cys-Asn-Thr-Xaa3-Thr-Xaa1-Xaa5-Cys-Thr-Leu-His-Gln-His-Cys-Cys-Ser-Gly-His-Cys- | (SEQ ID NO:30) |
| His-Lys-Thr-Ile-Gln-Ala-Cys-Ala-\hat{ } | |
| Name: Ar6.8 | |
| Species: arenatus | |
| Cloned: Yes | |
| DNA Sequence: | |
| GGATCCATGAAACTGACGTGTGTGGTGATCATCGCCGTGCTGTTCCTGACGGCCTGT | (SEQ ID NO:31) |
| CAACTCACTACAGGTGAGCAGAAGGACCATGCTCTGAGGTCAACTGACAAAAACTC | |
| CAAGTTGACTAGGCAGTGCTCGCCTATCGGTGGATATTGTACTCTTCATATTCACTG | |
| CTGCAGCAACCATTGCATTAAACCTATCGGCCGATGTGTGGCAACCTGATACCCGTG | |
| CGTGGTCATGAACCCCTCAATACCCTCTCCTCTGGAGGCTTCAGAGGAACTGCATTG | |
| AAATAAAACCGCATTGCAATAAAAAAAAAAAAAAAAAA | |
| Translation: | |
| MKLTCVVIIAVLFLTACQLTTGEQKDHALRSTDKNSKLTRQCSPIGGYCTLHIHCCSNHC | (SEQ ID NO:32) |
| IKPIGRCVAT | |
| Toxin Sequence: | |
| Xaa2-Cys-Ser-Xaa3-Ile-Gly-Gly-Xaa5-Cys-Thr-Leu-His-Ile-His-Cys-Cys-Ser-Asn-His-Cys- | (SEQ ID NO:33) |
| Ile-Lys-Xaa3-Ile-Gly-Arg-Cys-Val-Ala-Thr-\hat{ } | |
| Name: Ar6.9 | |
| Species: arenatus | |
| Cloned: Yes | |
| DNA Sequence: | |
| GGATCCATGAAACTGACGTGCGTGGTGATCATCGCCGTGCTGTTCCTGACGGCCTGT | (SEQ ID NO:34) |
| CAACTCACTACAGGTGAGCAGAAGGACCATGCTCTGAGGTCAACTGACAAAAACTC | |
| CAAGTTGACTAGGCAGTGCTTGCCTAACGGTGGATATTGTACTCTTCATATTCACTG | |
| CTGCAGCGACCATTGCATTAAACCTATCGACCGATGTGTGGCAACCTGATACCCGG | |
| GCGTGGTCATGAACCCCTCAATACCCTCTCCTCTGGAGGCTTCAGAGGAACTGCATT | |
| GAAATAAAACCGCATTACAAAAAAAAAAAAAAAAA | |
| Translation: | |
| MKLTCVVIIAVLFLTACQLTTGEQKDHALRSTDKNSKLTRQCLPNGGYCTLHIHCCSDH | (SEQ ID NO:35) |
| CIKPIDRCVAT | |
| Toxin Sequence: | |
| Xaa2-Cys-Leu-Xaa3-Asn-Gly-Gly-Xaa5-Cys-Thr-Leu-His-Ile-His-Cys-Cys-Ser-Asp-His-Cys- | (SEQ ID NO:36) |
| Ile-Lys-Xaa3-Ile-Asp-Arg-Cys-Val-Ala-Thr-\hat{ } | |
| Name: Ay6.1 | |
| Species: aurisiacus | |
| Cloned: Yes | |
| DNA Sequence: | |
| ATGAAACTGACGTGTGTGGTGATCGTCGCCGTGCTGCTCCTGACGGCCTGTCAACTC | (SEQ ID NO:37) |
| ATCACAGCTGATGACTCCAGAGGTACGCAGAAGCATCGTTCCCTGAGCTCGGCCAC | |
| CAAACTCTCCATGTCGACTCGCTGCAAGGGTAAAGGAAAACCATGCAGTAGGATTT | |
| CGTATAACTGCTGCACCGGTTCTTGCAGATCAGGTAAATGTGGCTGATCCAGCGCCT | |
| GATCTTCCCCCTTCTGTGCTCTATCCTTTTCTGCCTGAGTCCTCCTTACCTGAGAGTG | |
| GTCATGAACCACTCATCACCTGCTCCTCTGGAGGCCCCAGAGGAGCTACATTGAAAT | |
| AAAAGTCGCATTGCAAAAAAAAAAAAAAAAAAA | |
| Translation: | |
| MKLTCVVIVAVLLLTACQLITADDSRGTQKHRSLSSATKLSMSTRCKGKGKPCSRISYN | (SEQ ID NO:38) |
| CCTGSCRSGKCG | |
| Toxin Sequence: | |
| Cys-Lys-Gly-Lys-Gly-Lys-Xaa3-Cys-Ser-Arg-Ile-Ser-Xaa5-Asn-Cys-Cys-Thr-Gly-Ser-Cys- | (SEQ ID NO:39) |
| Arg-Ser-Gly-Lys-Cys-# | |
| Name: Ay6.2 | |
| Species: aurisiacus | |
| Cloned: Yes | |
| DNA Sequence: | |
| ATGAAACTGACGTGTGTGGTGATCGTCGCCGTGCTGCTCCTGACGGCCTGTCAACTC | (SEQ ID NO:37) |
| ATCACAGCTGATGACTCCAGAGGTACGCAGAAGCATCGTTCCCTGAGCTCGGCCAC | |
| CAAACTCTCCATGTCGACTCGCTGCAAGGGTAAAGGAAAACCATGCAGTAGGATTT | |
| CGTATAACTGCTGCACCGGTTCTTGCAGATCAGGTAAATGTGGCTGATCCAGCGCCT | |
| GATCTTCCCCCTTCTGTGCTCTATCCTTTTCTGCCTGAGTCCTCCTTACCTGAGAGTG | |
| GTCATGAACCACTCATCACCTGCTCCTCTGGAGGCCCCAGAGGAGCTACATTGAAAT | |
| AAAAGTCGCATTGCAAAAAAAAAAAAAAAAAAA | |
| Translation: | |
| MKLTCVVIVAVLLLTACQLITADDSRGTQKHRSLSSATKLSMSTRCKGKGKPCSRISYN | (SEQ ID NO:38) |
| CCTGSCRSGKCG | |
| Toxin Sequence: | |
| Cys-Lys-Gly-Lys-Gly-Lys-Xaa3-Cys-Ser-Arg-Ile-Ser-Xaa5-Asn-Cys-Cys-Thr-Gly-Ser-Cys- | (SEQ ID NO:39) |
| Arg-Ser-Gly-Lys-Cys-# | |
| Name: Ay6.2 | |
| Species: aurisiacus | |
| Cloned: Yes | |
| DNA Sequence: | |
| ATGAAACTGACGTGTGTGGTGATCGTCGCCGTGCTGCTCCTGACGGCCTGTCAACTC | (SEQ ID NO:40) |
| ATCACAGCTGATGACTCCAGAGGTACGCAGAAGCATCGTTCCCTGAGGTCGAAGAC | |
| CAAACTCTCCATGTCGACTGGCTGCATGGAAGCCGGATCTTATTGCGGCTCTACTAC | |
| GAGAATCTGCTGCGGTTTTTGCGCTTATTTCGGCAAAAAATGTATTGACTATCCCAG | |
| CAACTGATCTTCCCCCTACTGTGCTCTATCCTTTTCTGCCTGAGTCCTCCTTACCTGA | |
| GAGTGGTCATGAACCACTCATCACCTGCTCCTCTGGAGGCCCCAGAGGAGCTACATT | |
| GAAATAAAATCGCATTGCTAAAAAAAAAAAAAAAAAAA | |
| Translation: | |
| MKLTCVVIVAVLLLTACQLITADDSRGTQKHRSLRSKTKLSMSTGCMEAGSYCGSTTRI | (SEQ ID NO:41) |
| CCGFCAYFGKKCIDYPSN | |
| Toxin Sequence: | |
| Cys-Met-Xaa1-Ala-Gly-Ser-Xaa5-Cys-Gly-Ser-Thr-Thr-Arg-Ile-Cys-Cys-Gly-Phe-Cys-Ala- | (SEQ ID NO:42) |
| Xaa5-Phe-Gly-Lys-Lys-Cys-Ile-Asp-Xaa5-Xaa3-Ser-Asn-\hat{ } | |
| Name: Ay6.3 | |
| Species: aurisiacus | |
| Cloned: Yes | |
| DNA Sequence: | |
| ACCAAAACCATCATCAAAATGAAACTGACGTGTGTGGTGATCGTCGCCGTGCTGCT | (SEQ ID NO:43) |
| CCTGACGGCCTGTCAACTCATCACAGCTGATGACTCCAGAGGTACGCAGAAGCATC | |
| GTTCCCTGAGCTCGGCCACCAAACTCTCCATGTCGACTCGCTGCAAGGCTAAAGGA | |
| AAACCATGCAGTAGGATTGCGTATAACTGCTGCACCGGTTCTTGCAGATCAGGTAA | |
| ATGTGGCTGATCCAGTGCCTGATCTTCCCCCTTCTGTGCTCTATCCTTTTCTGCCTGA | |
| GTCCTCCTTACCTGAGAGTGGTCATGAACCACTCATCACCTGCTCCTCTGGAGGCCC | |
| CAGAGGAGCTACATTGAAATAAAAGCCGCATTGC | |
| Translation: | |
| MKLTCVVIVAVLLLTACQLITADDSRGTQKHRSLSSATKLSMSTRCKAKGKPCSRIAYN | (SEQ ID NO:44) |
| CCTGSCRSGKCG | |
| Toxin Sequence: | |
| Cys-Lys-Ala-Lys-Gly-Lys-Xaa3-Cys-Ser-Arg-Ile-Ala-Xaa5-Asn-Cys-Cys-Thr-Gly-Ser-Cys- | (SEQ ID NO:45) |
| Arg-Ser-Gly-Lys-Cys-# | |
| Name: Ay6.4 | |
| Species: aurisiacus | |
| Cloned: Yes | |
| DNA Sequence: | |
| ACCAAAACCATCATCAAAATGAAACTGACGTGTGTGGTGATCGTCGCCGTGCTGCT | (SEQ ID NO:46) |
| CCTGACGACCTGTCAACTCATCACAGCTGATGACTCCAGAGGTACGCAGGAGCATC | |
| GTGCCCTGAGGTCGAAGACAAAACTCTCCATGTTAACTTTGCGCTGCGCATCTTACG | |
| GAAAACCTTGTGGTATTGACAACGACTGCTGCAATGCATGCGATCCAGGAAGAAAT | |
| ATATGTACGTAGCTGATCCAGCGCCTGATCTTCCCCCTTCTGTGCTCTATCCTTTTCT | |
| GCCCGAGTCCTCCTTACCTGAGAGTGGTCATGAACCACTCATCACCTGCTCCCTGGA | |
| GGCCTCAGAGGAGCTACAATGAAATAAAAGCCGCATTGC | |
| Translation: | |
| MKLTCVVIVAVLLLTTCQLITADDSRGTQEHRALRSKTKLSMLTLRCASYGKPCGIDND | (SEQ ID NO:47) |
| CCNACDPGRNICT | |
| Toxin Sequence: | |
| Cys-Ala-Ser-Xaa5-Gly-Lys-Xaa3 -Cys-Gly-Ile-Asp-Asn-Asp-Cys-Cys-Asn-Ala-Cys-Asp-Xaa3- | (SEQ ID NO:48) |
| Gly-Arg-Asn-Ile-Cys-Thr-\hat{ } | |
| Name: Bu6.1 | |
| Species: bullatus | |
| Cloned: Yes | |
| DNA Sequence: | |
| ACCAAAACCATCATCAAAATGAAACTGACGTGTGTGGCGATCGTCGCCGTGCTGCT | (SEQ ID NO:49) |
| CCTGACGGCCTGTCAGCTCATTACAGCTGAAGACTCCAGAGGTACGCATGAGCATC | |
| TTGCCCTGAAGTCGACCTCCAAAGTCTCCAAGTCGACTAGCTGCATGGAAGCCGGA | |
| TCTTATTGCGGACCTGCTACTACGAAAATCTGCTGCGATTTTTGCAGTCCATTCAGC | |
| GATAGATGTATGAACAATCCCAACAATTGATCTTCCCCCTTGTGTGCTCCATCCTTTT | |
| CTGCCTGAGTCCTCCTTACCTGAGAGTGGTCATGAACCACTCATCACCTACTCCTCT | |
| GGAGGCTTCAGAGGAGCTACATTGAAATAAAAGCCGCATTGC | |
| Translation: | |
| MKLTCVAIVAVLLLTACQLITAEDSRGTHEHLALKSTSKVSKSTSCMEAGSYCGPATTK | (SEQ ID NO:50) |
| ICCDFCSPFSDRCMNNPNN | |
| Toxin Sequence: | |
| Ser-Thr-Ser-Cys-Met-Xaa1-Ala-Gly-Ser-Xaa5-Cys-Gly-Xaa3-A1a-Thr-Thr-Lys-Ile-Cys-Cys- | (SEQ ID NO:51) |
| Asp-Phe-Cys-Ser-Xaa3-Phe-Ser-Asp-Arg-Cys-Met-Asn-Asn-Xaa3-Asn-Asn-\hat{ } | |
| Name: Bu6.2 | |
| Species: bullatus | |
| Cloned: Yes | |
| DNA Sequence: | |
| ACCAAAACCATCATCAAAATGAAACTGACGTGTGTGGTGATCGTCGCCGTGCTGCT | (SEQ ID NO:52) |
| CCTGACGGCCTGTCAGCTCATTACAGCTGAAGACTCCAGAGGTACGCAGTTGCATC | |
| GTGCCCTGAGGAAGGCCACCAAACACCCTGTGTCGACTCGCTGCATTACTCCAGGA | |
| ACACGATGTAAGGTTCCGAGCCAATGCTGCAGAGGTCCTTGCAAGAACGGTCGTTG | |
| TACTCCATCCCCTTCTGAATGGTAAATGTGGTTGATCCAGCGCCTGATCTTCCCCCTT | |
| CGTCGTGCTCCATCCTTTTCTGCCTGAGTCCTCCTTACCTGAGAGTGGTCATGAACC | |
| ACTCATCACCTACTCCCCTGGAGGCTTCAGAGGAGCTACATTGAAATAAAAGCCGC | |
| ATTGC | |
| Translation: | |
| MKLTCVVIVAVLLLTACQLITAEDSRGTQLHRALRKATKHPVSTRCITPGTRCKVPSQC | (SEQ ID NO:53) |
| CRGPCKNGRCTPSPSEW | |
| Toxin Sequence: | |
| Cys-Ile-Thr-Xaa3-Gly-Thr-Arg-Cys-Lys-Val-Xaa3-Ser-Gln-Cys-Cys-Arg-Gly-Xaa3-Cys-Lys- | (SEQ ID NO:54) |
| Asn-Gly-Arg-Cys-Thr-Xaa3-Ser-Xaa3-Ser-Xaa1-Xaa4-\hat{ } | |
| Name: Bu6.3 | |
| Species: bullatus | |
| Cloned: Yes | |
| DNA Sequence: | |
| ACCAAAACCATCATCAAAATGAAACTGACGTGTGTGGCGATCGTCGCCGTGCTGCT | (SEQ ID NO:55) |
| CCTGACGGCCTGTCAGCTCATTACAGCTGAGGACTCCAGAGATACGCAGAAGCATC | |
| GTGCCCTGAGGTCGGACACCAAACTCTCCATGTTGACTTTGCGCTGCGCAACTTACG | |
| GAAAACCTTGTGGTATTCAAAACGACTGCTGCAATACATGCGATCCAGCCAGAAGG | |
| ACATGTACGTAGCTGATCCGGCGTCTTGATCCTCCGCTTCTGTGCTCCATCTTTTCTG | |
| CCTGAGTCCTCCTTACCTGAGAGTGGTCATGAACCACTCATCACCTACTCCTCTGGA | |
| GGCTTTAGAGGAGCTACATTGAAATAAAAGCCGCATTGC | |
| Translation: | |
| MKLTCVAIVAVLLLTACQLITAEDSRDTQKHRALRSDTKLSMLTLRCATYGKPCGIQND | (SEQ ID NO:56) |
| CCNTCDPARRTCT | |
| Toxin Sequence: | |
| Cys-Ala-Thr-Xaa5-Gly-Lys-Xaa3-Cys-Gly-Ile-Gln-Asn-Asp-Cys-Cys-Asn-Thr-Cys-Asp-Xaa3- | (SEQ ID NO:57) |
| Ala-Arg-Arg-Thr-Cys-Thr-\hat{ } | |
| Name: Bu6.4 | |
| Species: bullatus | |
| Cloned: Yes | |
| DNA Sequence: | |
| ACCAAAACCATCATCAAAATGAAACTGACGTGTGTGGCGATCGTCGCCGTGCTGCT | (SEQ ID NO:58) |
| CCTGACGGCCTGTCAGCTCATTACAGCTGAAGACTCCAGAGGTACGCAGTTGCATC | |
| GTGCCCTGAGGAAGACCACCAAACTCTCCTTGTCGACTCGCTGCAAGGGTCCAGGA | |
| GCATCATGTATAAGGATTGCGTATAACTGCTGCAAGTATTCTTGCAGAAATGGTAAA | |
| TGTGGCTGATCCAGCGCCTGATCTTCCCCCTTGTGTGCTCCATCCTTTTCTGCCTGAG | |
| TCCTCCTTACCTGAGAGTGGTCATGAACCACTCATCACCTACTCCTCTGGAGGCTTC | |
| AGAGGAGCTACATTGAAATAAAAGCCGCATTGC | |
| Translation: | |
| MKLTCVAIVAVLLLTACQLITAEDSRGTQLHRALRKTTKLSLSTRCKGPGASCIRIAYNC | (SEQ ID NO:59) |
| CKYSCRNGKCG | |
| Toxin Sequence: | |
| Cys-Lys-Gly-Xaa3-Gly-Ala-Ser-Cys-Ile-Arg-Ile-Ala-Xaa5-Asn-Cys-Cys-Lys-Xaa5-Ser-Cys- | (SEQ ID NO:60) |
| Arg-Asn-Gly-Lys-Cys-# | |
| Name: Bu6.5 | |
| Species: bullatus | |
| Cloned: Yes | |
| DNA Sequence: | |
| ATCAAAACCATCATCAAAATGAAACTGACGTGTGTGGTGATCGTCGCCGTGCTGCTC | (SEQ ID NO:61) |
| CTGACGGCCTGTCAGCTCATTACAGCTGAAGACTCCAGAGGTACGCATGAGCATCTT | |
| GCCCTGAAGTCGACCTCCAAAGTCTCCAAGTCGACTAGCTGCATGGCAGCCGGATC | |
| TTATTGCGGACCTGCTACTACGAATATCTGCTGCGATTTTTGCAGTCCATTCAGCGA | |
| TAGATGTATGAAAAAGCCCAACAATTGATCTTCCCCCTTCTGTGCTCTATCCTTTTCT | |
| GCCTGAGTCCTCCTTACCTGAGAGTGGTCATGAACCACTCATCACCTACTCCTCTGG | |
| AGGCTTCAGAGGAGCTACATTGAAATAAAAGCCGCATTGC | |
| Translation: | |
| MKLTCVVIVAVLLLTACQLITAEDSRGTHEHLALKSTSKVSKSTSCMAAGSYCGPATTN | (SEQ ID NO:62) |
| ICCDFCSPFSDRCMKKPNN | |
| Toxin Sequence: | |
| Ser-Thr-Ser-Cys-Met-Ala-Ala-Gly-Ser-Xaa5-Cys-Gly-Xaa3-Ala-Thr-Thr-Asn-Ile-Cys-Cys- | (SEQ ID NO:63) |
| Asp-Phe-Cys-Ser-Xaa3-Phe-Ser-Asp-Arg-Cys-Met-Lys-Lys-Xaa3-Asn-Asn-\hat{ } | |
| Name: Bu6.6 | |
| Species: bullatus | |
| Cloned: Yes | |
| DNA Sequence: | |
| ACCAAAACCATCATCAAAATGAAACTGACGTGTGTGGTGATCGTCGCCGTGCTGCT | (SEQ ID NO:64) |
| CCTGACGGCCTGTCAGCTCATTATAGCTGAGGACTCCAGAGGTACGCAGTTGCATCG | |
| TGCCCTGAGGAAGGCCACCAAACTCTCCGTGTCGACTCGCTGCAAGAGTAAAGGAT | |
| CATCATGTCATAGGACTTCGTATGACTGCTGCACGGGTTCTTGCAGAAATGGTAGAT | |
| GTGGCTGATCCAGCGCCTGATCTTCCCCCTTCTGTGCTCCATCCTTTTCTGCCTGAGT | |
| CCTCCTTACCTGAGAGTGGTCATGAACCACTCATCACCTACTCCTCTGGAGGCTTCA | |
| GAGGAGCTACATTGAAATAAAAGCCGCATTGC | |
| Translation: | |
| MKLTCVVIVAVLLLTACQLIIAEDSRGTQLHRALRKATKLSVSTRCKSKGSSCHRTSYD | (SEQ ID NO:65) |
| CCTGSCRNGRCG | |
| Toxin Sequence: | |
| Cys-Lys-Ser-Lys-Gly-Ser-Ser-Cys-His-Arg-Thr-Ser-Xaa5-Asp-Cys-Cys-Thr-Gly-Ser-Cys-Arg- | (SEQ ID NO:66) |
| Asn-Gly-Arg-Cys-# | |
| Name: Ca6.4 | |
| Species: caracteristicus | |
| Cloned: Yes | |
| DNA Sequence: | |
| GGATCCATGAAACTGACGTGCGTGGTGATCATCGCCGTGCTGTTCCTGACGGCCTGT | (SEQ ID NO:67) |
| CAACTCATTACAGGTGAGCAGAAGGACCATGCTCTGAGGTCAACTGACAAAAACTC | |
| CAAGTTGACTAGGCAGTGCTCGGCTAACGGTGGATCTTGTACTCGTCATTTTCACTG | |
| CTGCAGCCTCTATTGCAATAAAGATTCCAGTGTATGTGTGGCAACCTCATACCCGTG | |
| AGTGGCCATGAACCCCTCAATACCCTCTCCTCTGGAGGCTTCAGAGGAACTGCATTG | |
| AAATAAAACCGCATTACAAAAAAAAAAAAAAAAAAAA | |
| Translation: | |
| MKLTCVVIIAVLFLTACQLITGEQKDHALRSTDKNSKLTRQCSANGGSCTRHFHCCSLY | (SEQ ID NO:68) |
| CNKDSSVCVATSYP | |
| Toxin Sequence: | |
| Xaa2-Cys-Ser-Ala-Asn-Gly-Gly-Ser-Cys-Thr-Arg-His-Phe-His-Cys-Cys-Ser-Leu-Xaa5-Cys- | (SEQ ID NO:69) |
| Asn-Lys-Asp-Ser-Ser-Val-Cys-Val-Ala-Thr-Ser-Xaa5-Xaa3-\hat{ } | |
| Name: C6.1 | |
| Species: catus | |
| Cloned: Yes | |
| DNA Sequence: | |
| Translation: | |
| CKSTGASCRRTSYDCCTGSCRSGRCG | (SEQ ID NO:70) |
| Toxin Sequence: | |
| Cys-Lys-Ser-Thr-Gly-Ala-Ser-Cys-Arg-Arg-Thr-Ser-Xaa5-Asp-Cys-Cys-Thr-Gly-Ser-Cys-Arg- | (SEQ ID NO:71) |
| Ser-Gly-Arg-Cys-# | |
| Name: C6.4 | |
| Species: catus | |
| Cloned: Yes | |
| DNA Sequence: | |
| TCGACTCGCTGCCAGGGTAGAGGAGCATCATGTCGTAAGACTATGTATAACTGCTG | (SEQ ID NO:72) |
| CAGCGGTTCTTGCAACAGAGGTAGTTGTGGCTGATCCGGCGCCTGATCTTCCCCCTT | |
| CCGTGCTCTATCCTTTTCTGCCTGATTCCTCCTTACCTGAGAGCGGTCATGAACCACT | |
| CATCACCTGCTCCTCTGGAGGCCTCAGAGGAGCTACATTGAAATAAAAGCCGCATT | |
| GC | |
| Translation: | |
| STRCQGRGASCRKTMYNCCSGSCNRGSCG | (SEQ ID NO:73) |
| Toxin Sequence: | |
| Cys-Gln-Gly-Arg-Gly-Ala-Ser-Cys-Arg-Lys-Thr-Met-Xaa5-Asn-Cys-Cys-Ser-Gly-Ser-Cys- | (SEQ ID NO:74) |
| Asn-Arg-Gly-Ser-Cys-# | |
| Name: C6.5 | |
| Species: catus | |
| Cloned: Yes | |
| DNA Sequence: | |
| TCGACACGCTGCTTGCCTGCCGGAGAGTCTTGCCTTTTTAGTAGGATTAGATGCTGC | (SEQ ID NO:75) |
| GGTACTTGCAGTTCAGTCTTAAAGTCATGTGTGAGCTGATCCAGCTGCTGATCTTCC | |
| TCCTCCTGTGCTCCATCCTTTTCTGCCTGAGTCCTCCTTATCTGAGAGTGGTCATGAA | |
| CCACTCACCACCTACTCTTCTGGAGGCTTCAGAGGAGCTACAGTGAAATAAAAGCC | |
| GCATTGC | |
| Translation: | |
| STRCLPAGESCLFSRIRCCGTCSSVLKSCVS | (SEQ ID NO:76) |
| Toxin Sequence: | |
| Cys-Leu-Xaa3-Ala-Gly-Xaa1-Ser-Cys-Leu-Phe-Ser-Arg-Ile-Arg-Cys-Cys-Gly-Thr-Cys-Ser- | (SEQ ID NO:77) |
| Ser-Val-Leu-Lys-Ser-Cys-Val-Ser-\hat{ } | |
| Name: C6.6 | |
| Species: catus | |
| Cloned: Yes | |
| DNA Sequence: | |
| TCGACACGCTGCCAGGGTAGAGGAGGACCATGTACTAAGGCTGTGTTTAACTGCTG | (SEQ ID NO:78) |
| CAGCGGTTCTTGCAACAGAGGTAGATGTGGCTGATCCAGCGCCTGATCTTCCCCCTT | |
| CTGTGCTCTATCCTTTTCTGCCTGAGTCCTCCTTACTGAGAGTAGTCATGAACCACTC | |
| ATCACCTACTCCTCTGGAGGCCTCAGAGAGCTACATTGAAATAAAAGCCGCATTGC | |
| Translation: | |
| STRCQGRGGPCTKAVFNCCSGSCNRGRCG | (SEQ ID NO:79) |
| Toxin Sequence: | |
| Cys-Gln-Gly-Arg-Gly-Gly-Xaa3-Cys-Thr-Lys-Ala-Val-Phe-Asn-Cys-Cys-Ser-Gly-Ser-Cys- | (SEQ ID NO:80) |
| Asn-Arg-Gly-Arg-Cys-# | |
| Name: C6.7 | |
| Species: catus | |
| Cloned: Yes | |
| DNA Sequence: | |
| TTAACTTTGCGCTGCGCAACTTACGGAAAACCTTGTGGTATTCAAAACGACTGCTGC | (SEQ ID NO:81) |
| AATACATGCGATCCAGCCAGAAAGACATGTACGTAGCTGATCCGGCGTCTGATCTC | |
| CCCCCTTCTGTGCTCTATCCTTTTCTGCCTGAGTCCTCCTTACCTGAGAGTGGTCATG | |
| AACCACTCATCACCTGCTCCTCTGGAGGCCTCGGGGGAGCTACATTGAAATAAAAG | |
| CCGCATTGC | |
| Translation: | |
| LTLRCATYGKPCGIQNDCCNTCDPARKTCT | (SEQ ID NO:82) |
| Toxin Sequence: | |
| Cys-Ala-Thr-Xaa5-Gly-Lys-Xaa3-Cys-Gly-Ile-Gln-Asn-Asp-Cys-Cys-Asn-Thr-Cys-Asp-Xaa3- | (SEQ ID NO:83) |
| Ala-Arg-Lys-Thr-Cys-Thr-\hat{ } | |
| Name: C6.8 | |
| Species: catus | |
| Cloned: Yes | |
| DNA Sequence: | |
| TCGACTCGCTGCCGGGGTAGAGGAGGACCATGTACTAAGGCTATGTTTAACTGCTG | (SEQ ID NO:84) |
| CAGCGGTTCTTGCAACAGAGGTAGATGTGGCTGATCCAGCGCCTGATCTTCCCCCTT | |
| CTGTGCTCTATCCTTTTCTGCCTGAGTCCTCCTTAACTGAGAGTAGTCATGAACCACT | |
| CATCACCTACTCCTCTGGAGGCCTCAGAGAAGCATCATTGAAATAAAAGCCGCATT | |
| GC | |
| Translation: | |
| STRCRGRGGPCTKAMFNCCSGSCNRGRCG | (SEQ ID NO:85) |
| Toxin Sequence: | |
| Cys-Arg-Gly-Arg-Gly-Gly-Xaa3-Cys-Thr-Lys-Ala-Met-Phe-Asn-Cys-Cys-Ser-Gly-Ser-Cys- | (SEQ ID NO:86) |
| Asn-Arg-Gly-Arg-Cys-# | |
| Name: Cr6.1 | |
| Species: circumcisus | |
| Cloned: Yes | |
| DNA Sequence: | |
| ACCAAAACCATCATCAAAATGAAACTGACGTGTGTGGTGATCGTCGCCGTGCTGCT | (SEQ ID NO:87) |
| CCTGACGACCTGTCAACTCATCACAGCTGATGACTCCAGAGGTACGCAGGAGCATC | |
| GTGCCCTGAGGTCGGACACCAAACTCCCCATGTCGACTCGCTGCAAGGGTAAAGGA | |
| GCATCATGTCGTAAGACTATGTATAACTGCTGCAGCGGTTCTTGCAGCAACGGTAGA | |
| TGTGGCTGATCCAGCGCCTGATCTTCCCCCTTCTGCTGCTCTATCCTTTTCTGCCTGA | |
| GTCCTCCTTACCTGAGAGCTGGTCATGAACCACTCATCACCTGCTCCTCTGGAGGCC | |
| CAGAGGAGCTACATTGAAATAAAAGCCGCATTGC | |
| Translation: | |
| MKLTCVVIVAVLLLTTCQLITADDSRGTQEHRALRSDTKLPMSTRCKGKGASCRKTMY | (SEQ ID NO:88) |
| NCCSGSCSNGRCG | |
| Toxin Sequence: | |
| Cys-Lys-Gly-Lys-Gly-Ala-Ser-Cys-Arg-Lys-Thr-Met-Xaa5-Asn-Cys-Cys-Ser-Gly-Ser-Cys-Ser- | (SEQ ID NO:89) |
| Asn-Gly-Arg-Cys-# | |
| Name: Cr6.2 | |
| Species: circumeisus | |
| Cloned: Yes | |
| DNA Sequence: | |
| ACCAAAACCATCATCAAAATGAAACTGACGTGTGTGGTGATCGTCGCCGTGCTGCT | (SEQ ID NO:90) |
| CCTGACGACCTGTCAACTCATCACAGCTGATGACTCCAGAGGTACGCAGAAGCATC | |
| GTGCCCTGAGGTCGGCCACCAAAGTCTCCAAGTCGACTAGCTGCATGGAAGCCGGA | |
| TCTTATTGCCGCTCTACTACGAGAACCTGCTGCGGTTATTGCTCTTATTTCAGCAAAA | |
| AATGTATTGACTTTCCCAGCAACTGATCTTCCCCCTACTGTGCTCTATCCTTTTCTGC | |
| CTGAGTCCTCCTTACCTGAGAGTGGTCATGAACCACTCATCACCCTACTCCTCTGGA | |
| GGCCCAGAGGAGCTACATTGAAATAAAAGCCGCATTGC | |
| Translation: | |
| MKLTCVVIVAVLLLTTCQLITADDSRGTQKHRALRSATKVSKSTSCMEAGSYCRSTTRT | (SEQ ID NO:91) |
| CCGYCSYFSKKCIDFPSN | |
| Toxin Sequence: | |
| Ser-Thr-Ser-Cys-Met-Xaa1-Ala-Gly-Ser-Xaa5-Cys-Arg-Ser-Thr-Thr-Arg-Thr-Cys-Cys-Gly- | (SEQ ID NO:92) |
| Xaa5-Cys-Ser-Xaa5-Phe-Ser-Lys-Lys-Cys-Ile-Asp-Phe-Xaa3-Ser-Asn-\hat{ } | |
| Name: Cr6.3 | |
| Species: circumcisus | |
| Cloned: Yes | |
| DNA Sequence: | |
| ACCAAAACCATCATCAAAATGAAACTGACGTGTGTGGTGATCGTCGCCGTGCTGCT | (SEQ ID NO:93) |
| CCTGACGACCTGTCAACTCATCACAGCTGATGACTCCAGAGGTACGCAGGAGCATC | |
| GTGCCCTGAGGTCGGACACCAAACTCCCCATGTCGACTCGCTGCAAGAGTAAAGGA | |
| GCAAAATGTTCAAGGCTTATGTATGACTGCTGCAGCGGTTCTTGCAGCAGGTACTCA | |
| GGTAGATGTGGCTGATCCAGCGCCTGATCTTCCCCCTTCTGCTGCTCTATCCTTTTCT | |
| GCCTGAGTCCTCCTTACCTGAGAGTGGTCATGAACCACTCATCACCTACTCCTCTGG | |
| AGGCCCAGAGGAGCTACATTGAAATAAAAGCCGCATTGC | |
| Translation: | |
| MKLTCVVIVAVLLLTTCQLITADDSRGTQEHRALRSDTKLPMSTRCKSKGAKCSRLMY | (SEQ ID NO:94) |
| DCCSGSCSRYSGRCG | |
| Toxin Sequence: | |
| Cys-Lys-Ser-Lys-Gly-Ala-Lys-Cys-Ser-Arg-Leu-Met-Xaa5-Asp-Cys-Cys-Ser-Gly-Ser-Cys-Ser- | (SEQ ID NO:95) |
| Arg-Xaa5-Ser-Gly-Arg-Cys-# | |
| Name: Cr6.4 | |
| Species: circumcisus | |
| Cloned: Yes | |
| DNA Sequence: | |
| ACCAAAACCATCATCAAAATGAAACTGACGTGTGTGGTGATCGTCGCCGTGCTGCT | (SEQ ID NO:96) |
| CCTGACGACCTGTCAACTCATCACAGCTGATGACTCCAGAGGTACGCAGAAGCATC | |
| GTTCCCTGACGTCGGCCACCAAAGTCTCCAAGTCGACTGGCTGCATGAAAGCCGGA | |
| TCTTATTGCCGCTCTACTACGAGAACTTGCTGCGGTTATTGCGCTTATTTCGGCAAA | |
| AAATGTATTGACTATCCCAGCAACTGATCTTCCCCCTACTGTGCTCTATCCTTTTCTG | |
| CCTAAGTCCTCCTTACCTGAGAGTGGTCATGAACCACTCATCACCCTACTCCTCTGG | |
| AGGCCCAGAGGAGCTACATTGAAATAAAAGCCGCATTGC | |
| Translation: | |
| MKLTCVVIVAVLLLTTCQLITADDSRGTQKHRSLTSATKVSKSTGCMKAGSYCRSTTRT | (SEQ ID NO:97) |
| CCGYCAYFGKKCIDYPSN | |
| Toxin Sequence: | |
| Ser-Thr-Gly-Cys-Met-Lys-Ala-Gly-Ser-Xaa5-Cys-Arg-Ser-Thr-Thr-Arg-Thr-Cys-Cys-Gly- | (SEQ ID NO:98) |
| Xaa5-Cys-Ala-Xaa5-Phe-Gly-Lys-Lys-Cys-Ile-Asp-Xaa5-Xaa3-Ser-Asn-\hat{ } | |
| Name: Cn6.1 | |
| Species: consors | |
| Cloned: Yes | |
| DNA Sequence: | |
| ATGAAACTGACGTGTGTGGTGATCGTCGCCGTGCTGCTCCTGACGGCCTGTCAACTC | (SEQ ID NO:99) |
| CTCACAGCTGATGACTCCAGAGGTACGCAGAAGCATCGTGCCCTGAAGTCTTACAC | |
| CAAACTCTCCATGTTAACTTTGCGCTGCGCATCTTACGGAAAACCTTGTGGTATTGA | |
| CAACGACTGCTGCAATACATGCGATCCAGCCAGAAAGACATGTACGTAGCTGATCC | |
| GGCGTCTGATCTTCCCCCTTCTGTGCTCTATCCTTTTCTGCCTGAGTCCTCCTTACCT | |
| GAGAGTGGTCATGAACCACTCATCACCTAGCTCCTCTGGAGGCTTCAGAGGAGCTA | |
| CAATGAAATAAAAGCGCATTGC | |
| Translation: | |
| MKLTCVVIVAVLLLTACQLLTADDSRGTQKHRALKSYTKLSMLTLRCASYGKPCGIDN | (SEQ ID NO:100) |
| DCCNTCDPARKTCT | |
| Toxin Sequence: | |
| Cys-Ala-Ser-Xaa5-Gly-Lys-Xaa3-Cys-Gly-Ile-Asp-Asn-Asp-Cys-Cys-Asn-Thr-Cys-Asp-Xaa3- | (SEQ ID NO:101) |
| Ala-Arg-Lys-Thr-Cys-Thr-\hat{ } | |
| Name: Cn6.2 | |
| Species: consors | |
| Cloned: Yes | |
| DNA Sequence: | |
| ATGAAACTGACGTGTGTGGTGATCGTCGCCGTGCTGCTCCTGACGGCCTGTCAACTC | (SEQ ID NO:102) |
| CTCACAGCTGATGACTCCAGAGGTACGCAGAAGCATCGTGCCCTGAGGTCGGACAC | |
| CAAACTCTCCATGTCGACTCGCTGCAAGGGTACAGGAAAACCATGCAGTAGGATTG | |
| CGTATAACTGCTGCACCGGTTCTTGCAGATCAGGTAAATGTGGCTGATCCAGCGCCT | |
| GATCTCCCCCC | |
| Translation: | |
| MKLTCVVIVAVLLLTACQLLTADDSRGTQKHRALRSDTKLSMSTRCKGTGKPCSRIAY | (SEQ ID NO:103) |
| NCCTGSCRSGKCG | |
| Toxin Sequence: | |
| Cys-Lys-Gly-Thr-Gly-Lys-Xaa3-Cys-Ser-Arg-Ile-Ala-Xaa5-Asn-Cys-Cys-Thr-Gly-Ser-Cys- | (SEQ ID NO:104) |
| Arg-Ser-Gly-Lys-Cys-# | |
| Name: Cn6.3 | |
| Species: consors | |
| Cloned: Yes | |
| DNA Sequence: | |
| ATGAAACTGACGTGTGTGGTGATCGTCGCCGTGCTGCTCCTGACGGCCTGTCAACTC | (SEQ ID NO:105) |
| ATCACAGCTGATGACTCCAAAGGTACGCAGAAGCATCGTTCCCTGAGGTCGACCAC | |
| CAAAGTCTCCAAGGCGACTGACTGCATTGAAGCCGGAAATTATTGCGGACCTACTG | |
| TTATGAAAATCTGCTGCGGCTTTTGCAGTCCATACAGCAAAATATGTATGAACTATC | |
| CCCAAAATTGATCTTCCCCCTTCTGTGCTCTATCCTTTTCTGCCTGAGTCCTCCTTAC | |
| CTGAGAGTGGTCATGAACCACTCATCACCTCGTCCC | |
| Translation: | |
| MKLTCVVIVAVLLLTACQLITADDSKGTQKHRSLRSTTKVSKATDCIEAGNYCGPTVM | (SEQ ID NO:106) |
| KICCGFCSPYSKICMNYPQN | |
| Toxin Sequence: | |
| Ala-Thr-Asp-Cys-Ile-Xaa1-Ala-Gly-Asn-Xaa5-Cys-Gly-Xaa3-Thr-Val-Met-Lys-Ile-Cys-Cys- | (SEQ ID NO:107) |
| Gly-Phe-Cys-Ser-Xaa3-Xaa5-Ser-Lys-Ile-Cys-Met-Asn-Xaa5-Xaa3-Gln-Asn-\hat{ } | |
| Name: Cn6.4 | |
| Species: consors | |
| Cloned: Yes | |
| DNA Sequence: | |
| ATGAAACTGACGTGTGTGGTGATCGTCGCCGTGCTGCTCCTGACGGCCTGTCAACTC | (SEQ ID NO:108) |
| CTCACAGCTGATGACTCCAGAGGTACGCAGAAGCATCGTGCCCTGAGGTCGGACAC | |
| CAAACTCTCCATGTCGACTCGCTGCAAAGGTAAAGGAGCATCATGTACAAGGCTTA | |
| TGTATGACTGCTGCCACGGTTCTTGCAGCAGCAGCAAGGGTAGATGTGGCTGATCC | |
| GGCGCCTGATCTTCCCCCTTCTGTGCTCTATCCTTTTCTGCCTGAGTCCTCCTTACCT | |
| GAGAGGTGGTCATGAACCACTCATCACCTGCTCCCCTG | |
| Translation: | |
| MKLTCVVIVAVLLLTACQLLTADDSRGTQKHRALRSDTKLSMSTRCKGKGASCTRLM | (SEQ ID NO:19) |
| YDCCHGSCSSSKGRCG | |
| Toxin Sequence: | |
| Cys-Lys-Gly-Lys-Gly-Ala-Ser-Cys-Thr-Arg-Leu-Met-Xaa5-Asp-Cys-Cys-His-Gly-Ser-Cys- | (SEQ ID NO:110) |
| Ser-Ser-Ser-Lys-Gly-Arg-Cys-# | |
| Name: Cn6.5 | |
| Species: consors | |
| Cloned: Yes | |
| DNA Sequence: | |
| GGATCCATGAAACTGACGTGCATGGTGATCGTCGCCGTGCTGCTCCTGACGGCCTGT | (SEQ ID NO:111) |
| CAACTCATCACAGCTGATGACTCCAGAGGTACGCAGAAGCATCGTGCCCTGAGGTC | |
| GGACACCAAACTCTCCATGTCAACTCGCTGCAAGGGTAAAGGAGCATCATGTCATA | |
| GGACTTCGTATGACTGCTGCACCGGTTCTTGCAACAGAGGTAAATGTGGCTGATCCG | |
| GCGCCTGATCTTCCCCCTTCTGTGCTCTATCCTTTTCTGCCTGAGTCATCCATACCTG | |
| TGCTCGAG | |
| Translation: | |
| MKLTCMVIVAVLLLTACQLITADDSRGTQKHRALRSDTKLSMSTRCKGKGASCHRTSY | (SEQ ID NO:112) |
| DCCTGSCNRGKCG | |
| Toxin Sequence: | |
| Cys-Lys-Gly-Lys-Gly-Ala-Ser-Cys-His-Arg-Thr-Ser-Xaa5-Asp-Cys-Cys-Thr-Gly-Ser-Cys-Asn- | (SEQ ID NO:113) |
| Arg-Gly-Lys-Cys-# | |
| Name: Cn6.6 | |
| Species: consors | |
| Cloned: Yes | |
| DNA Sequence: | |
| GGATCCATGAAACTGACGTGCGTGGTGATCGTCGCCGTGCTGCTCCTGACGGCCTGT | (SEQ ID NO:114) |
| CAACTCATCACAGCTGATGACTCCAGAGGTACGCAGAAGCATCGTGCCCTGAAGTC | |
| GGACACCAAACTCTCCATGTTAACTTTGCGCTGCGCATCTTACGGAAAACCTTGTGG | |
| TATTTACAACGACTGCTGCAATACATGCGATCCAGCCAGAAAGACATGTACGTAGC | |
| TGATCCGGCGTCTGATCTTCCCCCTTCTGTGCTCTATCCTTTTCTGCCTGAGTCATCC | |
| ATACCTGTGCTCGAG | |
| Translation: | |
| MKLTCVVIVAVLLLTACQLITADDSRGTQKHRALKSDTKLSMLTLRCASYGKPCGIYN | (SEQ ID NO:115) |
| DCCNTCDPARKTCT | |
| Toxin Sequence: | |
| Cys-Ala-Ser-Xaa5-Gly-Lys-Xaa3-Cys-Gly-Ile-Xaa5-Asn-Asp-Cys-Cys-Asn-Thr-Cys-Asp- | (SEQ ID NO:116) |
| Xaa3-Ala-Arg-Lys-Thr-Cys-Thr-\hat{ } | |
| Name: Cn6.7 | |
| Species: consors | |
| Cloned: Yes | |
| DNA Sequence: | |
| GGATCCATGAAACTGACGTGTGTGGTGATCGTCGCCGTGCTGCTCCTGACGGCCTGT | (SEQ ID NO:117) |
| CAACTCATCACAGCTGATGACTCCAGAGGTACGCAGAAGCATCGTGCCCTGAGGTC | |
| GGACACCAAACTCTCCATGTCGACTCGCTGCAAGGGTACAGGAAAACCATGCAGTA | |
| GGGTTGCGTATAACTGCTGCACCGGTTCTTGCAGATCAGGTAAATGTGGCTGATCCA | |
| GTGCCTGATCTTCCCCCTTCTGTGCTCTATCCTTTTCTGCCTGAGTCCTCCTTACCTG | |
| AGAGTGGTCATGAACCACTCATCACCTGCTCCTCTGGAGGCTTCAGAGGAGCTACAT | |
| TGAAATAAAAGCCGCATTGCANTGNANAAAANNNNNNNNNNNNNNNNNNNNNNN | |
| NNNNNNNNNNNNNNNNNGGAAAAAAAAAAAAAAAAAAAA | |
| Translation: | |
| MKLTCVVIVAVLLLTACQLITADDSRGTQKHRALRSDTKLSMSTRCKGTGKPCSRVAY | (SEQ ID NO:118) |
| NCCTGSCRSGKCG | |
| Toxin Sequence: | |
| Cys-Lys-Gly-Thr-Gly-Lys-Xaa3-Cys-Ser-Arg-Val-Ala-Xaa5-Asn-Cys-Cys-Thr-Gly-Ser-Cys- | (SEQ ID NO:119) |
| Arg-Ser-Gly-Lys-Cys-# | |
| Name: Cn6.8 | |
| Species: consors | |
| Cloned: Yes | |
| DNA Sequence: | |
| GGATCCATGAAACTGACGTGCATGGTGATCGTCGCCGTGCTGCTCCTGACGGCCTGT | (SEQ ID NO:120) |
| CAACTCATCACAGCTGATGACTCCAGAGGTACGCAGAAGCATCGTTCCCTGAGGTC | |
| GACCACCAAAGTCTCCAAGTCGACTAGCTGCATGAAAGCCGGGTCTTATTGCCGCTC | |
| TACTACGAGAACCTGCTGCGGTTATTGCGCTTATTTCGGCAAATTTTGTATTGACTTT | |
| CCCAGCAACTGATCTTCCCCCTACTGTGCTCTATCCTTTTCTGCCTCTGCCTGAGTCC | |
| TCCTTACCTGAGAGTGGTCATGAACCACTCATCACCTGCTCCCCTGGAGGCCTCAGA | |
| GGAGCTACAATGAAATAAAAGCCGCATTGCAAAAAAAAAAAAAAAAAAAA | |
| Translation: | |
| MKLTCMVIVAVLLLTACQLITADDSRGTQKHRSLRSTTKVSKSTSCMKAGSYCRSTTRT | (SEQ ID NO:121) |
| CCGYCAYFGKFCIDFPSN | |
| Toxin Sequence: | |
| Ser-Thr-Ser-Cys-Met-Lys-Als-Gly-Ser-Xaa5-Cys-Arg-Ser-Thr-Thr-Arg-Thr-Cys-Cys-Gly- | (SEQ ID NO:122) |
| Xaa5-Cys-Ala-Xaa5-Phe-Gly-Lys-Phe-Cys-Ile-Asp-Phe-Xaa3-Ser-Asn-\hat{ } | |
| Name: Da6.8 | |
| Species: dalli | |
| Cloned: Yes | |
| DNA Sequence: | |
| ACCAAAACCATCATCAAAATGAAACTGACGTGTGTGGTGATCGTCGCCGTGCTGTTC | (SEQ ID NO:123) |
| CTGACGGCCTGTCAACTCATCACAGCTGATGACTCCAGAAGTACGCAGAAGCATCG | |
| TGCTCTGAGGTCGACCATCAAACACTCCATGTTGACTAGGAGCTGCACGCCTCCCGG | |
| AGGACCTTGTGGTTATTATAATGACTGCTGCAGTCATCAATGCAATATAAGCAGAA | |
| ATAAATGCGAGTAGCTGATCCGGCATCTGATCTTCCCCTTCTGTGCTCGTCCTAACC | |
| TGAGAGTGGTCATGAACCATCATCACCTACTCCTCTGGAGGCTTCAGAGGAGCTAC | |
| ATGGAAATAAAAGCCGCATTGC | |
| Translation: | |
| MRLTCVVIVAVLFLTACQLITADDSRSTQKHRALRSTIKHSMLTRSCTPPGGPCGYYND | (SEQ ID NO:124) |
| CCSHQCNISRNKCE | |
| Toxin Sequence: | |
| Ser-Cys-Thr-Xaa3-Xaa3-Gly-Gly-Xaa3-Cys-Gly-Xaa5-Xaa5-Asn-Asp-Cys-Cys-Ser-His-Gln- | (SEQ ID NO:125) |
| Cys-Asn-Ile-Ser-Arg-Asn-Lys-Cys-Xaa1-\hat{ } | |
| Name: Di6.1 | |
| Species: distans | |
| Cloned: Yes | |
| DNA Sequence: | |
| ACCAAAACCATCATCAAAATGAAACTGACGTGCGTGTTGATCATCGCCGTGCTGTTC | (SEQ ID NO:126) |
| CTGACGGCCTGTCAACTCACTAGAGGAAAGCTGGAGCGTCCTGTTCTGAGGTCGAG | |
| CGACCAAACCTCCGGGTCAACGAAGAGATGCGAAGATCCTGGTGAACCTTGCGGAA | |
| GTGATCATTCCTGCTGCGGCGGTAGTTGCAACCACAACGTCTGCGCCTGAAGCTGGT | |
| CTGGCATCTGACCATTCCCCTTCTGTACTCTATCTCTATTGCCTGAGTCATCTTTACC | |
| TGTGAGTGGTCATGAATCTCTCAATACCTTCTCCCCTGGAGGCTTCAGAAGAACTAG | |
| ATTGAAATA | |
| Translation: | |
| MKLTCVLIIAVLFLTACQLTRGKLERPVLRSSDQTSGSTKRCEDPGEPCGSDHSCCGGSC | (SEQ ID NO:127) |
| NHNVCA | |
| Toxin Sequence: | |
| Cys-Xaa1-Asp-Xaa3-Gly-Xaa1-Xaa3-Cys-Gly-Ser-Asp-His-Ser-Cys-Cys-Gly-Gly-Ser-Cys- | (SEQ ID NO:128) |
| Asn-His-Asn-Val-Cys-Ala-\hat{ } | |
| Name: E6.2 | |
| Species: ermineus | |
| Cloned: Yes | |
| DNA Sequence: | |
| ATGAAACTGACGTGTGTGGTGATCGTCGCCGTGCTGCTCCTGACGGCCTGTCAACTC | (SEQ ID NO:129) |
| ATCACAGCTGACGACTCCAGACGTACGCAGAAGCATCGTGCCCTGAGGTCGACCAC | |
| CAAACGCGCCACGTCGAATCGCCCCTGCAAGCCGAAAGGACGAAAATGTTTTCCGC | |
| ATCAGAAGGACTGCTGCAATAAAACGTGCACCAGATCAAAATGTCCCTGATCTTCC | |
| CCCTTCTGTGCTGTATCCTTTTCTGCCTGAGTCCTCCTTACCTGAGAGTGGTCAGTAA | |
| CCACTCATCACCATCTCCTCTGGAGG | |
| Translation: | |
| MKLTCVVIVAVLLLTACQLITADDSRRTQKHRALRSTTKRATSNRPCKPKGRKCFPHQK | (SEQ ID NO:130) |
| DCCNKTCTRSKCP | |
| Toxin Sequence: | |
| Xaa3-Cys-Lys-Xaa3-Lys-Gly-Arg-Lys-Cys-Phe-Xaa3-His-Gln-Lys-Asp-Cys-Cys-Asn-Lys-Thr- | (SEQ ID NO:131) |
| Cys-Thr-Arg-Ser-Lys-Cys-Xaa3-\hat{ } | |
| Name: E6.3 | |
| Species: ermineus | |
| Cloned: Yes | |
| DNA Sequence: | |
| AACTCATCACAGCTGATGACTCCAGAGGTACGCAGAACGATCGTGCCCTGAGGTCG | (SEQ ID NO:132) |
| ACCACCAAACTCTCCATGCTGACTCGGGCCTGCTGGTCTTCCGGAACACCTTGTGGT | |
| ACTGATAGTTTATGCTGCGGTGGATGCAATGTATCCAAAAGTAAATGTAACTAGCTG | |
| ATTCGGCGTCTGAACTTCCCCCTTCTGTGCTCTATCCTTTTCTGCCCGAGTCCTCCAT | |
| ACCTGAGAATGGTCATGAACCACTCATCACCTACTCCTCTGGAGACCTCAGAAGAG | |
| CTACACTGAAATAAAAGCGCTTGC | |
| Translation: | |
| LITADDSRGTQNDRALRSTTKLSMLTRACWSSGTPCGTDSLCCGGCNVSKSKCN | (SEQ ID NO:133) |
| Toxin Sequence: | |
| Ala-Cys-Xaa4-Ser-Ser-Gly-Thr-Xaa3-Cys-Gly-Thr-Asp-Ser-Leu-Cys-Cys-Gly-Gly-Cys-Asn- | (SEQ ID NO:134) |
| Val-Ser-Lys-Ser-Lys-Cys-Asn-\hat{ } | |
| Name: G6.1 | |
| Species: geographus | |
| Cloned: Yes | |
| DNA Sequence: | |
| GGATCCATGAAACTGACGTGCGTGGTGATCGTCGCCGTGCTGCTCCTGACGGCCTGT | (SEQ ID NO:135) |
| CAACTCATCACAGCTGATGACTCCAGAGGTACGCAGAAGCATCGTGCCCTGGGGTC | |
| GACCACCGAACTCTCCTTGTCGACTCGCTGCAAGTCACCCGGATCTTCATGTTCACC | |
| GACTAGTTATAATTGCTGCAGGTCTTGCAATCCATACGCCAAAAGATGTTACGGCTA | |
| ATCCAGCGCCTGATCTTCCCCCTTCTGTGCTCTATCCCTTCCTGTCTGAGTCCTCCTT | |
| ACCTGAGAGTGGTCATGAACCACTCCTCACCTACTTCTCTGGAGGCTTCGGAGGAGC | |
| TACATTGAAATAAAAGCCGCATTGTAAAAAAAAAAAAAAAAAA | |
| Translation: | |
| MKLTCVVIVAVLLLTACQLITADDSRGTQKHRALGSTTELSLSTRCKSPGSSCSPTSYNC | (SEQ ID NO:136) |
| CRSCNPYAKRCYG | |
| Toxin Sequence: | |
| Cys-Lys-Ser-Xaa3-Gly-Ser-Ser-Cys-Ser-Xaa3-Thr-Ser-Xaa5-Asn-Cys-Cys-Arg-Ser-Cys-Asn- | (SEQ ID NO:137) |
| Xaa3-Xaa5-Ala-Lys-Arg-Cys-Xaa5-# | |
| Name: G6.2 | |
| Species: geographus | |
| Cloned: Yes | |
| DNA Sequence: | |
| GGATCCATGAAACTGACGTGTGTGGTGATCGTCGCCGTGCTGCTCCTGACGGCCTGT | (SEQ ID NO:138) |
| CAACTCATCACAGCTGATGACTCCAGAGGTACGCAGAAGCATCGTGCCCTGAGGTC | |
| GTCCACCAAACTCACCTTGTCGACTCGCTGCAAATCACCCGGAACTCCATGTTCAAG | |
| GGGTATGCGTGATTGCTGCACGCCTTGCTTGTTATACAGCAACAAATGTAGGCGCTA | |
| CTAACCCAGCGCCTGATCTTCCCCCTTCTGTGCTCTATTCCTTTCTGCCTGAGTCCTC | |
| CTTACCTGAAAGTGGTCATGAACCACTCATCACCTACTTCTCTGGAGGCTTCAGAAG | |
| AGCTACATTGAAATAAAAGCCGCATTGCAATGACAAAAAAAAAAAAAAAAAAAA | |
| Translation: | |
| MKLTCVVIVAVLLLTACQLITADDSRGTQKHRALRSSTKLTLSTRCKSPGTPCSRGMRD | (SEQ ID NO:139) |
| CCTPCLLYSNKCRRY | |
| Toxin Sequence: | |
| Cys-Lys-Ser-Xaa3-Gly-Thr-Xaa3-Cys-Ser-Arg-Gly-Met-Arg-Asp-Cys-Cys-Thr-Xaa3-Cys-Leu- | (SEQ ID NO:140) |
| Leu-Xaa5-Ser-Asn-Lys-Cys-Arg-Arg-Xaa5-\hat{ } | |
| Name: w-GVIA | |
| Species: geographus | |
| Cloned: Yes | |
| DNA Sequence: | |
| GGAATTCCGTTTCTGCGCTGCTTCCTTTGGCATCACCAAAACCATCATCAAAATGAA | (SEQ ID NO:141) |
| ACTGACGTGTGTGGTGATCGTCGCCGTGCTGCTCCTGACGGCCTGTCAACTCATCAC | |
| AGCTGATGACTCCAGAGGTACGCAGAAGCATCGTGCCCTGGGGTCGACCACCGAAC | |
| TCTCCTTGTCGACTCGCTGCAAGTCACCCGGATCTTCATGTTCACCGACTAGTTATA | |
| ATTGCTGCAGGTCTTGCAATCCATACACCAAAAGATGTTACGGCTAATCCAGCGCCT | |
| GATCTTCCCTGCTCTGAGTCCTCCTTACCTGAGAGTGGTCATGAACCACTCATCACC | |
| TACTTCTCTAGGCGGTTCGGAGGAGCTACATTGAAATAAAAGCCGCATTGCAAAAA | |
| AAAAAAAAAA | |
| Translation: | |
| MKLTCVVIVAVLLLTACQLITADDSRGTQKHRALGSTTELSLSTRCKSPGSSCSPTSYNC | (SEQ ID NO:142) |
| CRSCNPYTKRCYG | |
| Toxin Sequence: | |
| Cys-Lys-Ser-Xaa3-Gly-Ser-Ser-Cys-Ser-Xaa3-Thr-Ser-Xaa5-Asn-Cys-Cys-Arg-Ser-Cys-Asn- | (SEQ ID NO:143) |
| Xaa3-Xaa5-Thr-Lys-Arg-Cys-Xaa5-# | |
| Name: w-GVIB | |
| Species: geographus | |
| Isolated: Yes | |
| Toxin Sequence: | |
| Cys-Lys-Ser-Xaa3-Gly-Ser-Ser-Cys-Ser-Xaa3-Thr-Ser-Xaa5-Asn-Cys-Cys-Arg-Ser-Cys-Asn- | (SEQ ID NO:144) |
| Xaa3-Xaa5-Thr-Lys-Arg-Cys-Xaa5-Gly-# | |
| Name: w-GVIC | |
| Species: geographus | |
| Isolated: Yes | |
| Toxin Sequence: | |
| Cys-Lys-Ser-Xaa3-Gly-Ser-Ser-Cys-Ser-Xaa3-Thr-Ser-Xaa5-Asn-Cys-Cys-Arg-Ser-Cys-Asn- | (SEQ ID NO:145) |
| Xaa3-Xaa5-Thr-Lys-Arg-Cys-# | |
| Name: w-GVIIA | |
| Species: geographus | |
| Isolated: Yes | |
| Cloned: Yes | |
| DNA Sequence: | |
| CATCACAGCTGATGACTCCAGAGGTACGCAGAAGCATCGTGCCCTGAGGTCGTCCA | (SEQ ID NO:146) |
| CCAAACTCACCTTGTCGACTCGCTGCAAATCACCCGGAACTCCATGTTCAAGGGGTA | |
| TGCGTGATTGCTGCACGTCTTGCTTGTTATACAGCAACAAATGTAGGCGCTACTAAC | |
| CCAGCGCCTGATCTTCCCCCTTCTGTGCTCTATTCCTTTCTGCCTGAGTCCTCCTTAC | |
| CTGAAAGTGGTCATGAACCACTCATCACCTACTTCTCTGGAGGCTTCAGAAGAGCTA | |
| CATTGAAATAAAAGCCGCATTGCAATGAC | |
| Translation: | |
| ITADDSRGTQKHRALRSSTKLTLSTRCKSPGTPCSRGMRDCCTSCLLYSNKCRRY | (SEQ ID NO:147) |
| Toxin Sequence: | |
| Cys-Lys-Ser-Xaa3-Gly-Thr-Xaa3-Cys-Ser-Arg-Gly-Met-Arg-Asp-Cys-Cys-Thr-Ser-Cys-Leu- | (SEQ ID NO:148) |
| Leu-Xaa5-Ser-Asn-Lys-Cys-Arg-Arg-Xaa5-# | |
| Name: w-GVIIB | |
| Species: geographus | |
| Isolated: Yes | |
| Toxin Sequence: | |
| Cys-Lys-Ser-Xaa3-Gly-Thr-Xaa3-Cys-Ser-Arg-Gly-Met-Arg-Asp-Cys-Cys-Thr-Ser-Cys-Leu- | (SEQ ID NO:149) |
| Ser-Xaa5-Ser-Asn-Lys-Cys-Arg-Arg-Xaa5-# | |
| Name: La6.1 | |
| Species: laterculatus | |
| Cloned: Yes | |
| DNA Sequence: | |
| ACCAAAACCATCATCAAAATGAAACTGACGTGTGTGGTGATCGTCGCCGTGCTGCT | (SEQ ID NO:150) |
| CCTGACGGCCTGTCAACTCATCACCGCTGATGACTCCAGAGGTACGCAGAAGCATC | |
| GTGCCCTGAGGTCGACCACCAATCTCTCCATGCTGACTCGGAAGTGCTGGCCTTCCG | |
| GAAGCTATTGTCGTGCGAATAGTAAATGCTGCAGTGGATGCGATCGGAACAGAAAT | |
| AAATGTTACTAGCTGATTCGGCGTCTGAACTTCCTCCTTCTGTGCTCTATCCTTTTCT | |
| GCCCGAGTCCTCCATACCTGAGAGTGGTCATGAACCACTCAACTCCTACTCCTCTGG | |
| AGGCCTCAGAAGAGCTACATTGAAATAAAAGCCGCATTGC | |
| Translation: | |
| MKLTCVVIVAVLLLTACQLITADDSRGTQKHRALRSTTNLSMLTRKCWPSGSYCRANS | (SEQ ID NO:151) |
| KCCSGCDRNRNKCY | |
| Toxin Sequence: | |
| Lys-Cys-Xaa4-Xaa3-Ser-Gly-Ser-Xaa5-Cys-Arg-Ala-Asn-Ser-Lys-Cys-Cys-Ser-Gly-Cys-Asp- | (SEQ ID NO:152) |
| Arg-Asn-Arg-Asn-Lys-Cys-Xaa5-\hat{ } | |
| Name: La6.2 | |
| Species: laterculatus | |
| Cloned: Yes | |
| DNA Sequence: | |
| ACCAAAACCATCATCAAAATGAAACTGACGTGTGTGGTGATCGTCGCCGTGCTGCT | (SEQ ID NO:153) |
| CCTGACGGCCTGTCAACTCATCACAGCTGATGACTCCAGAGGTACGCAGAAGCATC | |
| GTGCCCTGAGGTCGACCACCAAACTCTCCATATCGACTCGCTGCCTTCCTCCCGGAT | |
| CATATTGTAAGGCGACAACGGAAGTCTGCTGCTCTTCTTGCCTTCAATTCGCTCAGA | |
| TATGTTCGGGTTGATCTTCCCTCTTCTGTGCTCTATCCTTTTCTGCCTGAGTCCTCCAT | |
| ACCTGAGAATGGTCATGAACCACTCAACATCTACTCCTCTGGAGGCCTCAGAAGAG | |
| CTATATTGAAATAAAAGCCGCATTGC | |
| Translation: | |
| MKLTCVVIVAVLLLTACQLITADDSRGTQKHRALRSTTKLSISTRCLPPGSYCKATTEVC | (SEQ ID NO:154) |
| CSSCLQFAQICSG | |
| Toxin Sequence: | |
| Cys-Leu-Xaa3-Xaa3-Gly-Ser-Xaa5-Cys-Lys-Ala-Thr-Thr-Xaa1-Val-Cys-Cys-Ser-Ser-Cys-Leu- | (SEQ ID NO:155) |
| Gln-Phe-Ala-Gln-Ile-Cys-Ser-# | |
| Name: La6.3 | |
| Species: laterculatus | |
| Cloned: Yes | |
| DNA Sequence: | |
| ACCAAAACCATCATCAAAATGAAACTGACGTGTGTGGTGATCGTCGCCGTGCTGCT | (SEQ ID NO:156) |
| CCTGACGGCCTGTCAACTCATCACAGCTGATGACTCCAGAGGTACGCAGAAGCATC | |
| GTGCCCTGAGGTCGACCACCAATCTCTCCATGTCGACTCGCTGCAAGTCTCCCGGAT | |
| CATCATGTAGCGTGTCTATGCGTAACTGCTGCACTTCTTGCAATTCACGCACCAAGA | |
| AATGTACGCGACGTGGCTGAACTTCCCCCTTCTGTGCTCTATCCTTTTCTGCCCGAGT | |
| CCTCCATACCTGAGAGTGGTCATGAACCACTCAACATCTACTCCTCTGGAGGCCTCA | |
| GAAGAGCTATATTGAAATAAAAGCCGCATTGC | |
| Translation: | |
| MKLTCVVIVAVLLLTACQLITADDSRGTQKHRALRSTTNLSMSTRCKSPGSSCSVSMRN | (SEQ ID NO:157) |
| CCTSCNSRTKKCTRRG | |
| Toxin Sequence: | |
| Cys-Lys-Ser-Xaa3-Gly-Ser-Ser-Cys-Ser-Val-Ser-Met-Arg-Asn-Cys-Cys-Thr-Ser-Cys-Asn-Ser- | (SEQ ID NO:158) |
| Arg-Thr-Lys-Lys-Cys-Thr-Arg-Arg-# | |
| Name: La6.4 | |
| Species: laterculatus | |
| Cloned: Yes | |
| DNA Sequence: | |
| ACCAAAACCATCATCAAAATGAAACTGACGTGTGTGGTGATCGTCGCCGTGCTGCT | (SEQ ID NO:159) |
| CCTGACGGCCTGTCAACTCATCACAGCTGATGACTCCAGAGGTACGCAGAAGCATC | |
| GTGCCCTGAGGTCGACAACCAAACTCTCCATGCTGACTCGGACCTGCTGGCCTTCCG | |
| GAACAGCTTGTGGTATTGATAGTAACTGCTGCAGTGGATGCAATGTATCCAGAAGT | |
| AAATGTAACTAGCTGATTCGGCGTCTAAACTTCCTCCTTCTGCCTGAGTCCTCCATA | |
| CCTGAGAGTGGTCATGAACCACATCATCACCTCATCTCTGGAGGCCTC | |
| Translation: | |
| MKLTCVVIVAVLLLTACQLITADDSRGTQKHRALRSTTKLSMLTRTCWPSGTACGIDSN | (SEQ ID NO:160) |
| CCSGCNVSRSKCN | |
| Toxin Sequence: | |
| Thr-Cys-Xaa4-Xaa3-Ser-Gly-Thr-Ala-Cys-Gly-Ile-Asp-Ser-Asn-Cys-Cys-Ser-Gly-Cys-Asn- | (SEQ ID NO:161) |
| Val-Ser-Arg-Ser-Lys-Cys-Asn-\hat{ } | |
| Name: La6.5 | |
| Species: laterculatus | |
| Cloned: Yes | |
| DNA Sequence: | |
| ACCAAAACCATCATCAAAATGAAACTGACGTGTGTGGTGATCGTCGCCGTGCTGCT | (SEQ ID NO:162) |
| CCTGACGGCCTGTCAACTCATCACAGCTGATGACTCCAGAGGTACGCAGAAGCATC | |
| GTGCCCTGAGGTCGACCACCAATCTCTCCATGCTGACTCGGAAGTGCTGGCCTTCCG | |
| GAAGCTATTGTCGTGCGAATAGTAAATGCTGCAGTGGATGCGATCGGAACAGAAGT | |
| AAATGTAACTAGCTGATTCGGCGTCTAAACTTCCTCCTTCTGCCTGAGTCCTCCATA | |
| CCTGAGAGTGGTCATGAACCACTCATCACCTACTCCTCTGGAGGCCTCAAAGGAGCT | |
| ACATTGAAATAAAAGCCGCATTGC | |
| Translation: | |
| MKLTCVVIVAVLLLTACQLITADDSRGTQKHRALRSTTNLSMLTRKCWPSGSYCRANS | (SEQ ID NO:163) |
| KCCSGCDRNRSKCN | |
| Toxin Sequence: | |
| Lys-Cys-Xaa4-Xaa3-Ser-Gly-Ser-Xaa5-Cys-Arg-Ala-Asn-Ser-Lys-Cys-Cys-Ser-Gly-Cys-Asp- | (SEQ ID NO:164) |
| Arg-Asn-Arg-Ser-Lys-Cys-Asn-\hat{ } | |
| Name: Lp6.1 | |
| Species: leopardus | |
| Cloned: Yes | |
| DNA Sequence: | |
| ATGAAACTGACGTGTGTGGTGATCGTAGCTGTGCTGTTCCTGACGGCCTGTCAACTC | (SEQ ID NO:165) |
| ACTACAGCTGACATCTCCAGAGGTACGCGGAAGCGTCGTGCTCTGAGGTCGACCAC | |
| CAAACTCTCCAGGTCGCTCTTTGAGTGCGCGCCTTCCGGTGGACGTTGTGGTTTTTTA | |
| AAGTCCTGCTGCGAAGGATATTGCGATGGGGAAAGCACTTCATGTGTGAGTGGCCC | |
| ATACAGCATCTGATCTTCCCGCCTTCAGTGCTCTATCCTTTTCTGCCTGAGTCCTCCA | |
| TACCTCTGAGCGGTCATGAACCACTCAACACCTACTCCTCTGGAGGCTTCAGGGAAC | |
| TATATTAAAATAAAGCCGCATTGCAACGAAANAAAAAAAAAAAAAAAAA | |
| Translation: | |
| MKLTCVVIVAVLFLTACQLTTADISRGTRKRRALRSTTKLSRSLFECAPSGGRCGFLKSC | (SEQ ID NO:166) |
| CEGYCDGESTSCVSGPYSI | |
| Toxin Sequence: | |
| Ser-Leu-Phe-Xaa1-Cys-Ala-Xaa3-Ser-Gly-Gly-Arg-Cys-Gly-Phe-Leu-Lys-Ser-Cys-Cys-Xaa1- | (SEQ ID NO:167) |
| Gly-Xaa5-Cys-Asp-Gly-Xaa1-Ser-Thr-Ser-Cys-Val-Ser-Gly-Xaa3-Xaa5-Ser-Ile-\hat{ } | |
| Name: Lp6.2 | |
| Species: leopardus | |
| Cloned: Yes | |
| DNA Sequence: | |
| ATGAAACTGACGTGTGTGGTGATCGTCGCTGTGCTGTTCCTGACGGCCTGTCAACTC | (SEQ ID NO:168) |
| ACTACAGCTGACATCTCCAGAGGTACGTGGAAGCATCGTGGTGTGGGGTCGACCAC | |
| CGGACTCTCCCCGTGGCCCTTGGACTGCACGGCTCCCAGTCAACCTTGTGGTTATTT | |
| TCCTAGGTGCTGTGGACATTGCGATGTACGCAGGGTATGTACGAGTGGCTGATCCG | |
| GCGTCTGATCTTTCCGCCTTCTGTGCTGTATCCTTTTCTGCCTGAGTCCTCCATACCC | |
| GTGAGTGGTCATGAACCACTCAACACCTACTCCTCTGGAGGCTTCAGAGGAACTAT | |
| ATTAAAATAAAGCCGCATTGCAATG | |
| Translation: | |
| MKLTCVVIVAVLFLTACQLTTADISRGTWKHRGVGSTTGLSPWPLDCTAPSQPCGYFPR | (SEQ ID NO:169) |
| CCGHCDVRRVCTSG | |
| Toxin Sequence: | |
| Xaa4-Xaa3-Leu-Asp-Cys-Thr-Ala-Xaa3-Ser-Gln-Xaa3-Cys-Gly-Xaa5-Phe-Xaa3-Arg-Cys-Cys- | (SEQ ID NO:170) |
| Gly-His-Cys-Asp-Val-Arg-Arg-Val-Cys-Thr-Ser-# | |
| Name: Lp6.3 | |
| Species: leopardus | |
| Cloned: Yes | |
| DNA Sequence: | |
| ATGAAACTGACGTGTGTGGTGATCGTCGCTGTGCTGTTCCTGACGGCCTGTCAACTC | (SEQ ID NO:171) |
| ACTACAGCTGACATCTCCAGAGGTACGCGGAAGCATCGTGCTCTGAGGTCGACCAC | |
| CAAACTCTCCAGGTCGCCCTCTAGGTGCATGTCTCCCGGTGGAATTTGTGGTGATTT | |
| TGGTGACTGCTGCGAAATTTGCAATGTGTACGGTATATGTGTGAGTGACTTACCCGG | |
| CATCTGATCTTTCCGCCTTCTGTGCTCTATCCTTTTCTGCCTGAGTCCTCCATACCCCT | |
| GAGTGGTCATGGACCACTCAACACCTACTCCTCTGGAGGCTTCAGAGGAACTACATT | |
| AAAATAAAGCCGCATTGCAAAAAAAAAAAAAAAAAAAAA | |
| Translation: | |
| MKLTCVVIVAVLFLTACQLTTADISRGTRKHRALRSTTKLSRSPSRCMSPGGICGDFGDC | (SEQ ID NO:172) |
| CEICNVYGICVSDLPGI | |
| Toxin Sequence: | |
| Cys-Met-Ser-Xaa3-Gly-Gly-Ile-Cys-Gly-Asp-Phe-Gly-Asp-Cys-Cys-Xaa1-Ile-Cys-Asn-Val- | (SEQ ID NO:173) |
| Xaa5-Gly-Ile-Cys-Val-Ser-Asp-Leu-Xaa3-Gly-Ile-\hat{ } | |
| Name: Lp6.4 | |
| Species: leopardus | |
| Cloned: Yes | |
| DNA Sequence: | |
| ATGAAACTGACGTGTGTGGTGATCGTCGCTGTGCTGTTCCTGACGGCCTGTCAACTC | (SEQ ID NO:174) |
| ACTACAGCTGATGATTCCAGAGGTACACGGAAGCATCGTGCTCTGAGGTCAACCAC | |
| CAAACTCTCCAGGTGGCCCAGGTACTGCGCGCCTCCCGGTGGAGCTTGTGGGTTTTT | |
| TGATCACTGCTGCGGATATTGCGAAACGTTTTACAATACGTGTAGATGAGTTGGCTG | |
| ATCCGGCGCTTGATCTTTCCGCCTTCTGTTGCTCTATCTTTTTCTGCCTGAGTCCTCCC | |
| ATACCCCGTTGAGTGGTCCATGAACCACTCCAACACCTACTCCCTCCTTGGAAGCTT | |
| CCAAAGGAAACGACATTTAAAATAAATTCCCCATTGCAATTGGAAAAAAAAAAAAA | |
| AAAAA | |
| Translation: | |
| MKLTCVVIVAVLFLTACQLTTADDSRGTRKHRALRSTTKLSRWPRYCAPPGGACGFFD | (SEQ ID NO:175) |
| HCCGYCETFYNTCR | |
| Toxin Sequence: | |
| Xaa5-Cys-Ala-Xaa3-Xaa3-Gly-Gly-Ala-Cys-Gly-Phe-Phe-Asp-His-Cys-Cys-Gly-Xaa5-Cys- | (SEQ ID NO:176) |
| Xaa1-Thr-Phe-Xaa5-Asn-Thr-Cys-Arg-\hat{ } | |
| Name: L6.1 | |
| Species: lynceus | |
| Cloned: Yes | |
| DNA Sequence: | |
| ATGAAACTGACGTGTGTGGTGATCGTCGCCGTGCTGCTCCTGACGGCCTGTCAACTC | (SEQ ID NO:177) |
| ATCACAGCTGATGACTCCAGACGTACACAGAAGCATCGTGCCCTGAGGTCGACCAC | |
| CAATCTCTCCATGTCGACTCGCTGCAAGTCTCCCGGATCACCATGTAGTGTGACATC | |
| GTATAACTGCTGCACTTTTTGCTCTTCATACACTAAGAAATGTCGGGCCTCTTTATGA | |
| ACCACTCATCACCTACTCCTCTGGAGGCCTCAGAAGAGCTACACTGAAATAAAAGC | |
| CGCATTGG | |
| Translation: | |
| MKLTCVVIVAVLLLTACQLITADDSRRTQKHRALRSTTNLSMSTRCKSPGSPCSVTSYN | (SEQ ID NO:178) |
| CCTFCSSYTKKCRASL | |
| Toxin Sequence: | |
| Cys-Lys-Ser-Xaa3-Gly-Ser-Xaa3-Cys-Ser-Val-Thr-Ser-Xaa5-Asn-Cys-Cys-Thr-Phe-Cys-Ser- | (SEQ ID NO:179) |
| Ser-Xaa5-Thr-Lys-Lys-Cys-Arg-Ala-Ser-Leu-\hat{ } | |
| Name: L6.2 | |
| Species: lynceus | |
| Cloned: Yes | |
| DNA Sequence: | |
| ATGAAACTGACGTGTGTGGTGATCGTCGCCGTGCTGCTCCTGACGGCCTGTCAACTC | (SEQ ID NO:180) |
| ATCACAGCTGATGACTCCAGAGGTACGCAGAAGCATCGTGCCCTGAGGTCGACCAC | |
| CAAACTATCCATGTATACTCGCTGCGCAGGTCCAGGAGCAATTTGTCCTAATAGGGT | |
| ATGCTGCGGTTATTGCAGTAAAAGAACACATCTATGTCATTCGCGAACTGGCTGATC | |
| TTCCCCCTTCTGTGCTCTATCCTTTTTCTGCCTGAGTCCTCCATACCTGAGAATGGTC | |
| ATGAACCACTCATCACCTACTCCTCTTGGAGACCTCAGAGGAGCTACACTGAAATA | |
| AAAGCCGCATTGGC | |
| Translation: | |
| MKLTCVVIVAVLLLTACQLITADDSRGTQKHRALRSTTKLSMYTRCAGPGAICPNRVCC | (SEQ ID NO:181) |
| GYCSKRTHLCHSRTG | |
| Toxin Sequence: | |
| Cys-Ala-Gly-Xaa3-Gly-Ala-Ile-Cys-Xaa3-Asn-Arg-Val-Cys-Cys-Gly-Xaa5-Cys-Ser-Lys-Arg- | (SEQ ID NO:182) |
| Thr-His-Leu-Cys-His-Ser-Arg-Thr-# | |
| Name: L6.3 | |
| Species: lynceus | |
| Cloned: Yes | |
| DNA Sequence: | |
| ATGAAACTGACGTGTGTGGTGATCGTCGCCGTGCTGCTGCTAGCGGCCTGTCAACTA | (SEQ ID NO:183) |
| CTACACGCTGATGACTCCAGAGGTACGCAGAAGACTGCTGCCCGAGGTCGACCACC | |
| AAAACTCTCCATGCTGACTCGGGCCTGCTGGTCTTCCGGAACACCTTGTGGTACTGA | |
| TAGTTTATGCTGCGGTGGATGCAATGTATCCAAAAGTAAATGTAACTAGCTGATTCG | |
| GCGTCTGAACTTCCCCCTTCTGTGCTCTATCCTTTTCTGCCCGAGTCCTCCATACCTG | |
| AGAATGGTCATGAACCACTCATCACCTACTCCTCTGGAGACCTCAGAAGAGCTACA | |
| CTGAAATAAAAGCGCATTGC | |
| Translation: | |
| MKLTCVVIVAVLLLAACQLLHADDSRGTQKTAAGRPPLSMLTRACCWSSGTPCGTDS | (SEQ ID NO:184) |
| LCCGGCNVSKSKCN | |
| Toxin Sequence: | |
| Ala-Cys-Xaa4-Ser-Ser-Gly-Thr-Xaa3-Cys-Gly-Thr-Asp-Ser-Leu-Cys-Cys-Gly-Gly-Cys-Asn- | (SEQ ID NO:185) |
| Val-Ser-Lys-Ser-Lys-Cys-Asn-\hat{ } | |
| Name: L6.4 | |
| Species: lynceus | |
| Cloned: Yes | |
| DNA Sequence: | |
| ATGAAACTGACGTGTGTGGTGATCGTCGCCGAGCTACTCCTAACGGCCTGTCAACTC | (SEQ ID NO:186) |
| ATCACAGCTGATGACTCCAGAGGTACGCAGAAGCATCGTGCCCTGAGGTCGACCAC | |
| CAATCTCTCCATGCTGACTCGGAAGTGCTGGTCTCCCGGAACCTATTGTCGTGCGCA | |
| TAGTAAATGCTGCCGTGGATGCGATCAGAACAGAAATAAATGTTACTAGCTGATTC | |
| GGCGTCTGAACTTCCTCCTTCTGTGCTCTATCCTTTTTCTGCCTGAGTCCTCCATACC | |
| TGAGAATGGTCATGAACCACTCATCACCTACTCCTCTGGAGGCCTCAGAGGAGCCT | |
| ACACTGAAATAAAAGCCGCATTGG | |
| Translation: | |
| MKLTCVVIVAELLLTACQLITADDSRGTQKHRALRSTTNLSMLTRKCWSPGTYCRAHS | (SEQ ID NO:187) |
| KCCRGCDQNRNKCY | |
| Toxin Sequence: | |
| Lys-Cys-Xaa4-Ser-Xaa3-Gly-Thr-Xaa5-Cys-Arg-Ala-His-Ser-Lys-Cys-Cys-Arg-Gly-Cys-Asp- | (SEQ ID NO:188) |
| Gln-Asn-Arg-Asn-Lys-Cys-Xaa5-\hat{ } | |
| Name: M6.1 | |
| Species: magus | |
| Cloned: Yes | |
| DNA Sequence: | |
| ACCAAAACCATCATCAAAATGAAACTGACGTGTGTGGTGATCGTCGCCGTGCTGCT | (SEQ ID NO:189) |
| CCTGACGGCCTGTCAACTCATCACAGCTGATGACTCCAGAGGTACGCAGAAGCATC | |
| GTGCCCTGAGGTCGGACACCAAACTCTCCATGTCGACTCGCTGCAAGGGTACAGGA | |
| AAACCATGCAGTAGGATTGCGTATAACTGCTGCACCGGTTCTTGCAGATCAGGTAA | |
| ATGTGGCTGATCCAGTGCCTGATCTTCCCCCTTCTGTGCTCTATCCTTTTTCTGCCTG | |
| AGTCCTCCTTACCTGAGAGTGGTCATGAACCACTCA | |
| Translation: | |
| MKLTCVVIVAVLLLTACQLITADDSRGTQKHRALRSDTKLSMSTRCKGTGKPCSRIAYN | (SEQ ID NO:190) |
| CCTGSCRSGKCG | |
| Toxin Sequence: | |
| Cys-Lys-Gly-Thr-Gly-Lys-Xaa3-Cys-Ser-Arg-Ile-Ala-Xaa5-Asn-Cys-Cys-Thr-Gly-Ser-Cys- | (SEQ ID NO:191) |
| Arg-Ser-Gly-Lys-Cys-# | |
| Name: M6.2 | |
| Species: magus | |
| Cloned: Yes | |
| DNA Sequence; | |
| ACCAAAACCATCATCAAAATGAAACTGACGTGTGTGGTGATCGTCGCCGTGCTGCT | (SEQ ID NO:192) |
| CCTGACGGCCTGTCAACTCATCACAGCTGATGACTCCAGAGGTACGCAGAAGCATC | |
| GTGCCCTGAAGTCGGACACCAAACTCTCCATGTTAACTTTGCGCTGCGCATCTTACG | |
| GAAAACCTTGTGGTATTTACAACGACTGCTGCAATACATGCGATCCAGCCAGAAAG | |
| ACATGTACGTAGCTGATCCGGCGTCTGATCTTCC | |
| Translation: | |
| MKLTCVVIVAVLLLTACQLITADDSRGTQKHRALKSDTRLSMLTLRCASYGKPCGIYN | (SEQ ID NO:193) |
| DCCNTCDPARKTCT | |
| Toxin Sequence: | |
| Cys-Ala-Ser-Xaa5-Gly-Lys-Xaa3-Cys-Gly-Ile-Xaa5-Asn-Asp-Cys-Cys-Asn-Thr-Cys-Asp- | (SEQ ID NO:194) |
| Xaa3-Ala-Arg-Lys-Thr-Cys-Thr-\hat{ } | |
| Name: w-MVIIB | |
| Species: magus | |
| Isolated: Yes | |
| Cloned: Yes | |
| DNA Sequence: | |
| GAATTTTCAGCATCACCAAAACCATCATCAAAATGAAACTGACGTGTGTGGTGATC | (SEQ ID NO:195) |
| GTCGCCGTGCTGCTCCTGACGGCCTGTCAACTCATCACAGCTGATGACTCCAGAGGT | |
| ACGCAGAAGCATCGTGCCCTGAGGTCGGACACCAAACTCTCCATGTCAACTCGCTG | |
| CAAGGGTAAAGGAGCATCATGTCATAGGACTTCGTATGACTGCTGCACCGGTTCTTG | |
| CAACAGAGGTAAATTTGGCTGATCCGCC | |
| Translation: | |
| MKLTCVVIVAVLLLTACQLITADDSRGTQKHRALRSDTKLSMSTRCKGKGASCHRTSY | (SEQ ID NO:196) |
| DCCTGSCNRGKFG | |
| Toxin Sequence: | |
| Cys-Lys-Gly-Lys-Gly-Ala-Ser-Cys-His-Arg-Thr-Ser-Xaa5-Asp-Cys-Cys-Thr-Gly-Ser-Cys-Asn- | (SEQ ID NO:197) |
| Arg-Gly-Lys-Cys-# | |
| Name: Mi6.1 | |
| Species: miles | |
| Cloned: Yes | |
| DNA Sequence: | |
| GGATCCATGAAACTGACGTGCGTGGTGATCATCGCCATGCTGTTCCTGACAGCCTAT | (SEQ ID NO:198) |
| CAACTCGCTACAGCTGCGAGCTACGCCAAAGGTAAACAGAAGCATCGTGCTCTGAG | |
| GCCAGCTGACAAACACCTCAGGTTGACCAAGCGTTGCAATGATCGCGGTGGAGGTT | |
| GCAGTCAACATCCTCACTGCTGCGGTGGAACTTGCAATAAGCTTATTGGCGTATGTC | |
| TGTAAAGCTGGTCTGCCGTCTGATATTCCCTTTCTGTGCTTCATCCTCTTTTGCCTGA | |
| GTCATCCATACCTGTGAATGGTTAAGAGCCACTCAATACCTATTCCTCTGGGGGCTT | |
| CAGAGGAACTACTTTAC | |
| Translation: | |
| MKLTCVVIIAMLFLTAYQLATAASYAKGKQKHRALRPADKHLRLTKRCNDRGGGCSQ | (SEQ ID NO:199) |
| HPHCCGGTCNKLIGVCL | |
| Toxin Sequence: | |
| Cys-Asn-Asp-Arg-Gly-Gly-Cys-Ser-Gln-His-Xaa3-His-Cys-Cys-Gly-Gly-Thr-Cys-Asn- | (SEQ ID NO:200) |
| Lys-Leu-Ile-Gly-Val-Cys-Leu-\hat{ } | |
| Name: Mn6.1 | |
| Species: monachus | |
| Cloned: Yes | |
| DNA Sequence: | |
| ACCAAAACCATCATCAAAATGAAACTGACGAGTGTGGTGATCGTCGCCGTGCTGCT | (SEQ ID NO:201) |
| CCTGACGGCCTGTCAACTCATCACAGCTGATGACTCCAGAGGTACGCAGAAGCATC | |
| GTGCCCTGAGGTCGGACACCAAACTCTCCATATCGACTCGCTGCAAGTCTACAGGA | |
| AAATCATGCAGTAGGATTGCGTATAACTGCTGCACCGGTTCTTGCAGATCAGGTAA | |
| ATGTGGCTGATCCAGCGCCTGATCTTCCCCCTTCTGTGCTCTATCCTTTTCTGCCTGA | |
| GTCCTCCTTA | |
| Translation: | |
| MKLTSVVIVAVLLLTACQLITADDSRGTQKHRALRSDTKLSISTRCKSTGKSCSRIAYNC | (SEQ ID NO:202) |
| CTGSCRSGKCG | |
| Toxin Sequence: | |
| Cys-Lys-Ser-Thr-Gly-Lys-Ser-Cys-Ser-Arg-Ile-Ala-Xaa5-Asn-Cys-Cys-Thr-Gly-Ser-Cys-Arg- | (SEQ ID NO:203) |
| Ser-Gly-Lys-Cys-# | |
| Name: Mn6.2 | |
| Species: monachus | |
| Cloned: Yes | |
| DNA Sequence: | |
| ACCAAAACCATCATCAAAATGAAACTGACGAGTGTGGTGATCGTCGCCGTGCTGCT | (SEQ ID NO:204) |
| CCTGACGGCCTGTCAACTCATCACAGCTGATGACTCCAGAGGTACGCAGAAGCATC | |
| GTGCCCTGAGGTCGGACACCAACCTCTCCATGTCGACTCGCTGCAAGGGTAAAGGA | |
| TCTTCATGTAGTAGGACCATGTATAACTGCTGCACCGGTTCTTGCAACAGAGGTAAA | |
| TGTGGCTGATCCAGCGCCTGATCTTCCCCCTTC | |
| Translation: | |
| MKLTSVVIVAVLLLTACQLITADDSRGTQKHRALRSDTNLSMSTRCKGKGSSCSRTMY | (SEQ ID NO:205) |
| NCCTGSCNRGKCG | |
| Toxin Sequence: | |
| Cys-Lys-Gly-Lys-Gly-Ser-Ser-Cys-Ser-Arg-Thr-Met-Xaa5-Asn-Cys-Cys-Thr-Gly-Ser-Cys- | (SEQ ID NO:206) |
| Asn-Arg-Gly-Lys-Cys-# | |
| Name: O6.1 | |
| Species: obscurus | |
| Cloned: Yes | |
| DNA Sequence: | |
| ctctctctctctctgctggacAGGTCGCCTCCCTGCATGAAAGGCGGATCGTCATGCCGCGGTAC | (SEQ ID NO:207) |
| TACGGGAGTCTGTTGCGGTTTTTGCAGTGATTTCGGCTATAAATGTAGGGACTATCC | |
| CCAAAACTGATCTTCCCCCTTCTGTGCTCTATCCTTTTCTGTCCGAGTCCTCCTGACC | |
| TGAGAGTGGTCATGAACCACTCATCACCTACCCCTCTGGGGCTTCACAGGATCTACA | |
| TTGAAATAAAAGCCGCATTGC | |
| Translation: | |
| LLDRSPPCMKGGSSCRGTTGVCCGFCSDFGYKCRDYPQN | (SEQ ID NO:208) |
| Toxin Sequence: | |
| Ser-Xaa3-Xaa3-Cys-Met-Lys-Gly-Gly-Ser-Ser-Cys-Arg-Gly-Thr-Thr-Gly-Val-Cys-Cys-Gly- | (SEQ ID NO:209) |
| Phe-Cys-Ser-Asp-Phe-Gly-Xaa5-Lys-Cys-Arg-Asp-Xaa5-Xaa3-Gln-Asn-\hat{ } | |
| Name: O6.2 | |
| Species: obscurus | |
| Cloned: Yes | |
| DNA Sequence: | |
| ctctctctctctctgctggacAGGTCGACTCGCTGCTTGCCTGACGGAACGTCTTGCCTTTTTAGT | (SEQ ID NO:210) |
| AGGATCAGATGCTGCGGTACTTGCAGTTCAATCTTAAAGTCATGTGTGAGCTGATCC | |
| AGCGGTTGATCTTCCTCCCTCTGTGCTCCATCCTTTTCTGCCTGAGTTCTCCTTACCT | |
| GAGAGTGGTCATGAACCACTCATCACCTACTCTTCTGGAGGCTTCAGAGGAGCTAC | |
| ATTGAAATAAAAGCCGCATTGC | |
| Translation: | |
| RSTRCLPDGTSCLFSRIRCCGTCSSILKSCVS | (SEQ ID NO:211) |
| Toxin Sequence: | |
| Cys-Leu-Xaa3-Asp-Gly-Thr-Ser-Cys-Leu-Phe-Ser-Arg-Ile-Arg-Cys-Cys-Gly-Thr-Cys-Ser-Ser- | (SEQ ID NO:212) |
| Ile-Leu-Lys-Ser-Cys-Val-Ser-\hat{ } | |
| Name: Pu6.2 | |
| Species: pulicarius | |
| Cloned: Yes | |
| DNA Sequence: | |
| ATGAAACTGACGTGTGTGGTGATCATCGCCGTGCTGTTCCTGACGGCCTGTCAACTC | (SEQ ID NO:213) |
| ATTACAGCTGAGACTTACTCCAGAGGTAAGCAGAAGCACCGTGCTTTGAGGTCAAC | |
| TGACAAAAACTCCAAGTTGACTAGGCAGTGCTCGCCTAACGGTGGATCTTGTTCTCG | |
| TCATTTTCACTGCTGCAGCCTCTATTGCAATAAAAATACTGGCGTATGTATTGCAAC | |
| CTAATACCCGTGTGTGGTCATGAACCACTCAATACCCTCTCCTCTGGAGGCTTCAGA | |
| GGAACTGCATTGAAATAAAACTGCATTGCNTTGACCAAAAAAAAAA | |
| Translation: | |
| MKLTCVVIIAVLFLTACQLITAETYSRGKQKHRALRSTDKNSKLTRQCSPNGGSCSRHF | (SEQ ID NO:214) |
| HCCSLYCNKNTGVCIAT | |
| Toxin Sequence: | |
| Xaa2-Cys-Ser-Xaa3-Asn-Gly-Gly-Ser-Cys-Ser-Arg-His-Phe-His-Cys-Cys-Ser-Leu-Xaa5-Cys- | (SEQ ID NO:215) |
| Asn-Lys-Asn-Thr-Gly-Val-Cys-Ile-Ala-Thr-\hat{ } | |
| Name: P6.1 | |
| Species: purpurascens | |
| Cloned: Yes | |
| DNA Sequence: | |
| ATGAAACTGACGTGTGTGGTGATCGTCGCCGTGCTGTTCCTGACGGCCTGTCAACTC | (SEQ ID NO:216) |
| ATCACAGCTGATGACTCCAGACGTACGCAGAAGCATCGTGCCCTGAGGTCGACCAC | |
| CAAAGGCGCCACGTCGAATCGCCCCTGCAAGACACCCGGACGAAAATGTTTTCCGC | |
| ATCAGAAGGACTGCTGCGGTCGAGCGTGCATCATCACAATATGTCCCTGATCTTCCC | |
| CCTTCTGTGCTGTATCCTTTTCTGCCTGAGTCTCCTTACCTGAGAGTGGTCATGAA | |
| Translation: | |
| MKLTCVVIVAVLFLTACQLITADDSRRTQKHRALRSTTKGATSNRPCKTPGRKCFPHQK | (SEQ ID NO:217) |
| DCCGRACIITICP | |
| Toxin Sequence: | |
| Xaa3-Cys-Lys-Thr-Xaa3-Gly-Arg-Lys-Cys-Phe-Xaa3-His-Gln-Lys-Asp-Cys-Cys-Gly-Arg-Ala- | (SEQ ID NO:218) |
| Cys-Ile-Ile-Thr-Ile-Cys-Xaa3-\hat{ } | |
| Name: P6.2 | |
| Species: purpurascens | |
| Isolated: Yes | |
| Cloned: Yes | |
| DNA Sequence: | |
| ACCAAAACCATCATCAAAATGAAACTGACGTGTGTGGTGATCGTCGCCGTGCTGCT | (SEQ ID NO:219) |
| CCTGACGGCCTGTCAACTCATCACAGCTGATGACTCCAGAGGTACGCAGAAGCATC | |
| GTGCCCTGAGGTCGACCACCAAACTCTTCACGTCGAAAAGCTGCAAGCTTCCCGGA | |
| GCATATTGTAATGCAGAAGATTATGACTGCTGCCTTAGATGCAAAGTTGGAGGTAC | |
| ATGTGGCTGATCCAGTGCCTGATCTTCCCCCTTCTGTGCTCTATCCTTTTCTGCCTGA | |
| GTCCTCCTTACCTAAGAGTGGTCATGAACCACTCATCACCTTCTCCTCTGGAGGCTT | |
| C | |
| Translation: | |
| MKLTCVVIVAVLLLTACQLITADDSRGTQKHRALRSTTKLFTSKSCKLPGAYCNAEDYD | (SEQ ID NO:220) |
| CCLRCKVGGTCG | |
| Toxin Sequence: | |
| Ser-Cys-Lys-Leu-Xaa3-Gly-Ala-Xaa5-Cys-Asn-Ala-Xaa1-Asp-Xaa5-Asp-Cys-Cys-Leu-Arg- | (SEQ ID NO:221) |
| Cys-Lys-Val-Gly-Gly-Thr-Cys-# | |
| Name: P6.3 | |
| Species: purpurascens | |
| Cloned: Yes | |
| DNA Sequence: | |
| ATGAAACTGACGTGTGTGGTGATCGTCGCCGTGCTGTTCCTGACGGCCTGTCAACTC | (SEQ ID NO:222) |
| ATCACAGCTGATGACTCCAGACGTACGCAGAAGCATCGTGCCCTGAGGTCGACCAC | |
| CAAACGCGCCACGTCGAATCGCCCCTGCAAGAAAACCGGACGAAAATGTTTTCCGC | |
| ATCAGAAGGACTGCTGCGGTCGAGCGTGCATCATCACAATATGTCCCTGATCTTCCC | |
| CCTTCTGTGCTGTATCCTTTTCTGCCTGAGTCCTCCTTACCTGAGAGTGGTCATGAAC | |
| CACTCATCACCTTCTCCTCTGGAGGCTTCAGAG | |
| Translation: | |
| MKLTCVVIVAVLFLTACQLITADDSRRTQKHRALRSTTKRATSNRPCKKTGRKCFPHQK | (SEQ ID NO:223) |
| DCCGRACIITICP | |
| Toxin Sequence: | |
| Xaa3-Cys-Lys-Lys-Thr-Gly-Arg-Lys-Cys-Phe-Xaa3-His-Gln-Lys-Asp-Cys-Cys-Gly-Arg-Ala- | (SEQ ID NO:224) |
| Cys-Ile-Ile-Thr-Ile-Cys-Xaa3-\hat{ } | |
| Name: R6.1 | |
| Species: radiatus | |
| Cloned: Yes | |
| DNA Sequence: | |
| GCTGATGCCTGATCTTCATCGTTCTTCCCTGTCTCCTTTGGCATCACCAAAACCATCA | (SEQ ID NO:225) |
| TCAAAATGAAACTGACGTGTGTGGTGATCGTCGCCGTGCTGGTCCTGACGGCCTGTC | |
| AACTCATCACAGCTGATGACTCCAGAGGTATGCAGAAACATCATGCCCTGGGGTCG | |
| ATCAGCAGTCTCTTTAAGTCGACCCGTCATGGCTGCAAACCCCTCAAACGTCGTTGT | |
| TTCAATGATAAAGAATGCTGCAGCAAATTTTGCAATTCAGTCCGAAAGCAGTGTGG | |
| ATAAATGGCTAAAAAACTGAATAAAAGCCGCATTGCAAAAAAAA | |
| Translation: | |
| MKLTCVVIVAVLVLTACQLITADDSRGMQKHHALGSISSLFKSTRHGCKPLKRRCFNDK | (SEQ ID NO:226) |
| ECCSKFCNSVRKQCG | |
| Toxin Sequence: | |
| His-Gly-Cys-Lys-Xaa3-Leu-Lys-Arg-Arg-Cys-Phe-Asn-Asp-Lys-Xaa1-Cys-Cys-Ser-Lys-Phe- | (SEQ ID NO:227) |
| Cys-Asn-Ser-Val-Arg-Lys-Gln-Cys-# | |
| Name: R6.2 | |
| Species: radiatus | |
| Cloned: Yes | |
| DNA Sequence: | |
| GAAATGAAACTGACGTGTGTGGTGATCGTCGCCGTGCTGGTCCTGACGGCCTGTCA | (SEQ ID NO:228) |
| ACTCATCACAGCTGATGACTCCAGAGGTATGCAGAAACATCATGCCCTGGGGTCGA | |
| TCAGCAGTCTCTTTAAGTCGACCCGTCGTGGCTGCAAACCCCTCAAACGTCGTTGTT | |
| TCAATGATAAAGAATGCTGCAGCAAATTTTGCAATTCAGTCCGAAACCAGTGTGGA | |
| TAAATGGCTAAAAACTGAATAAAAG | |
| Translation: | |
| MKLTCVVIVAVLVLTACQLITADDSRGMQKHHALGSISSLFKSTRRGCKPLKRRCFNDK | (SEQ ID NO:229) |
| ECCSKFCNSVRNQCG | |
| Toxin Sequence: | |
| Arg-Gly-Cys-Lys-Xaa3-Leu-Lys-Arg-Arg-Cys-Phe-Asn-Asp-Lys-Xaa1-Cys-Cys-Ser-Lys-Phe- | (SEQ ID NO:230) |
| Cys-Asn-Ser-Val-Arg-Asn-GIn-Cys-# | |
| Name: w-RVIA | |
| Species: radiatus | |
| Cloned: Yes | |
| DNA Sequence: | |
| GGAATTCCGCTTGCACGGCGAACCTGACTTCATCTTTCTTCCCTGCCTCCTTTGGCAT | (SEQ ID NO:231) |
| CACCAAAACCATCATCAAAATGAAACTGACGTGTGTGGTGATCGTCGCCGTGCTGG | |
| TCCTGACGGCCTGTCAACTCATCACAGCTGATGACTCCAGAGGTATGCAGAAGCAT | |
| CATGCCCTGAGGTCGATCACCAAACTCTCCCTGTCGACTCGCTGCAAACCTCCCGGA | |
| TCACCATGTAGAGTTTCTTCGTATAACTGCTGCTCTTCTTGCAAATCATACAACAAG | |
| AAATGTGGCTGAACTTCCCCTTCTGTGCTCTATCCTTTTCCTGCCCGAGTCCTCCATA | |
| CCTGAGAGTAGTCATGAACCACTGATTACCTACTCCTCTGGAGGGCCTCAGAGGAG | |
| CTACTTTGAAATAAAAGCCCGCATTGCAAAAAAAAAA | |
| Translation: | |
| MRLTCVVIVAVLVLTACQLITADDSRGMQKHHALRSITKLSLSTRCKPPGSPCRVSSYN | (SEQ ID NO:232) |
| CCSSCKSYNKKCG | |
| Toxin Sequence: | |
| Cys-Lys-Xaa3-Xaa3-Gly-Ser-Xaa3-Cys-Arg-Val-Ser-Ser-Xaa5-Asn-Cys-Cys-Ser-Ser-Cys-Lys- | (SEQ ID NO:233) |
| Ser-Xaa5-Asn-Lys-Lys-Cys-Gly-# | |
| Name: Ra6.1 | |
| Species: rattus | |
| Cloned: Yes | |
| DNA Sequence: | |
| GGATCCATGAAACTGACGTGCATGGTGATCATCGCCGTGCTGTTCCTGACAGCCTGT | (SEQ ID NO:234) |
| CAATTCGATACAGCTGCGAGCTACGACAAAGGTAAGCAGAAACCTCCTACTCTGAG | |
| GCCAGCTGACAAACACATCAGGTTGACCAAGCGTTGCAATGCTCGCAATGATGGTT | |
| GCAGTCAACATTCTCAATGCTGCAGTGGATCTTGCAATAAGACTGCAGGCGTATGTC | |
| TGTAAAGCTGGTCTGCCGTCTGATATTCCCTTTCTGTGCTTTATCCTCTTTTGCCTGA | |
| GTCATCCATACCTGTGAATGGTTAAGAGCCACTCAATACCTACTCCTCTGGGGGCTT | |
| CAGAGGAACTACATTAAATAAAGCCACATTGCAAAAAAAAAAAAAAAAAAA | |
| Translation: | |
| MKLTCMVIIAVLFLTACQFDTAASYDKGKQKPPTLRPADKHIRLTKRCNARINDGCSQH | (SEQ ID NO:235) |
| SQCCSGSCNKTAGVCL | |
| Toxin Sequence: | |
| Cys-Asn-Ala-Arg-Asn-Asp-Gly-Cys-Ser-Gln-His-Ser-Gln-Cys-Cys-Ser-Gly-Ser-Cys-Asn-Lys- | (SEQ ID NO:236) |
| Thr-Ala-Gly-Val-Cys-Leu-\hat{ } | |
| Name: Ra6.2 | |
| Species: rattus | |
| Cloned: Yes | |
| DNA Sequence: | |
| GGATCCATGAAACTGACGTGCGTGGTGATCATCGCCGTGCTGTTCCTGACAGCCTGT | (SEQ ID NO:237) |
| CAACTCGATGCAGCTGCGAGCTACGACAAAGGTAAGCAGAAACCTCCTACTCTGAG | |
| GCCAGCTGACAAACACTTCAGGTTGATCAAGCGTTGCAATGCTCGCAATAGTGGTT | |
| GCAGTCAACATCCTCAATGCTGCAGTGGATCTTGCAATAAGACTGCAGGCGTATGTC | |
| TGTAAAGCTGGTCTGCCGTCTGATATTCCCTTTCTGTGCTTTATCCTCTTTTGCCTGA | |
| GTCATCCATACCTGTGAATGGTTAAGAGCCACTCAATACCTACTCCTCTGGGGGCTT | |
| CAGAGGAACTACATTAAATAAAGCCACATTGCAACGAAAAAAAAAAAAAAAAAA | |
| Translation: | |
| MKLTCVVIIAVLFLTACQLDAAASYDKGKQKPPTLRPADKHFRLIKRCNARNSGCSQHP | (SEQ ID NO:238) |
| QCCSGSCNTAGVCL | |
| Toxin Sequence: | |
| Cys-Asn-Ala-Arg-Asn-Ser-Gly-Cys-Ser-Gln-His-Xaa3-Gln-Cys-Cys-Ser-Gly-Ser-Cys-Asn- | (SEQ ID NO:239) |
| Lys-Thr-Ala-Gly-Val-Cys-Leu-\hat{ } | |
| Name: Ra6.3 | |
| Species: rattus | |
| Cloned: Yes | |
| DNA Sequence: | |
| GGATCCATGAAACTGACGTGTGTGGTGATCATCGCCGTGCTGTTCCTGACAGCCTGT | (SEQ ID NO:240) |
| CAATTCGATACAGCTGCGAGCTACGACAAAGGTAAGCAGAAACCTCCTACTCTGAG | |
| GCCAGCTGACAAACACTTCAGGTTGATCAAGCGTTGCAATGCTCGCAATAGTGGTT | |
| GCAGTCAACATCCTCAATGCTGCAGTGGATCTTGCAATAAGACTTTGGGCGTATGTC | |
| TGTAAAGCTGGTCTGCCGTCTGATATTCCCTTTCTGTGCTTTATCCTCTTTTGCCTGA | |
| GTCATCCATACCTGTGAATGGTTAAGAGCCACTCAATACCTACTCCTCTGGGGGCTT | |
| CAGAGGAACTACATTAAATAAAGCCACATTGAAAAAAAAAAAAAAAAAA | |
| Translation: | |
| MKLTCVVIIAVLFLTACQFDTAASYDKGKQKPPTLRPADKHFRLIKRCNARNSGCSQHP | (SEQ ID NO:241) |
| QCCSGSCNKTLGVCL | |
| Toxin Sequence: | |
| Cys-Asn-Ala-Arg-Asn-Ser-Gly-Cys-Ser-Gln-His-Xaa3-Gln-Cys-Cys-Ser-Gly-Ser-Cys-Asn- | (SEQ ID NO:242) |
| Lys-Thr-Leu-Gly-Val-Cys-Leu-\hat{ } | |
| Name: Sm6.1 | |
| Species: stercusmuscarum | |
| Cloned: Yes | |
| DNA Sequence: | |
| ACCAAAACCATCATCAAAATGAAACTGACGTGTGTGGTGATCGTCGCCGTGCTGCT | (SEQ ID NO:243) |
| CCTGACGGCCTGTCAACTCATCACAGCTGATGACTCCAGAGGTACGCAGAAGCATC | |
| GTGCCCTGAGGTCGAAGACCAAACTCTCCATGTCGACTCGCTGCAAGAGTAAAGGA | |
| GCAAAATGTTCAAGGCTTATGTATGACTGCTGCAGCGGTTCTTGCAGCGGCTACACA | |
| GGTAGATGTGGCTGATCCAGCGCCTGATCTTCCCCCTTCTGTGCTCTATCCTTTTCTG | |
| CCTGGGTCCTCCTTACCTGAGAGTGGTCATGAACCACTCATCACCTACTCCTCTGGA | |
| GGCCTCAGAGGAGTTACAATGAAATAAAAGCCGCATTGC | |
| Translation: | |
| MKLTCVVIVAVLLLTACQLITADDSRGTQKHRALRSKTKLSMSTRCKSKGAKCSRLMY | (SEQ ID NO:244) |
| DCCSGSCSGYTGRCG | |
| Toxin Sequence: | |
| Cys-Lys-Ser-Lys-Gly-Ala-Lys-Cys-Ser-Arg-Leu-Met-Xaa5-Asp-Cys-Cys-Ser-Gly-Ser-Cys-Ser- | (SEQ ID NO:245) |
| Gly-Xaa5-Thr-Gly-Arg-Cys-# | |
| Name: Sm6.2 | |
| Species: stercusmuscarum | |
| Isolated: Yes | |
| Toxin Sequence: | |
| Thr-Thr-Ser-Cys-Met-Gln-Ala-Gly-Ser-Xaa5-Cys-Gly-Ser-Thr-Thr-Arg-Ile-Cys-Cys-Gly-Xaa5- | (SEQ ID NO:246) |
| Cys-Ala-Xaa5-Phe-Gly-Lys-Lys-Cys-Ile-Asp-Xaa5-Xaa3-Ser-Asn-\hat{ } | |
| Name: Sm6.3 | |
| Species: stercusmuscarum | |
| Cloned: Yes | |
| DNA Sequence: | |
| ACCAAAACCATCATCAAAATGAAACTGACGTGTGTGGTGATCGTCGCCGTGCTGCT | (SEQ ID NO:247) |
| CCTGACGACCTGTCAACTCATCACAGCTGATGACTCCAGAGGTACGCAGGAGCATC | |
| GTGCCCTGAGGTCGAAGACCAAACTCTCCATGTTAACTTTGCGCTGCGCATCTTACG | |
| GAAAACCTTGTGGTATTGACAACGACTGCTGCAATGCATGCGATCCAGCCAGAAAT | |
| ATATGTACGTAGCTGATCCGGCGTCTGATCTTCCCCCTTCTGTGCTCTATCCTTTTCT | |
| GCCTGAGTCCTCCTTACCTGAGAGTGGTCATGAACCACTCATCATCTACTCTCCTGG | |
| AGGCCTCAGAGGAGCTACAATGAAATAAAAGCCGCATTGC | |
| Translation: | |
| MKLTCVVIVAVLLLTTCQLITADDSRGTQEHRALRSKTKLSMLTLRCASYGKPCGIDND | (SEQ ID NO:248) |
| CCNACDPARNICT | |
| Toxin Sequence: | |
| Cys-Ala-Ser-Xaa5-Gly-Lys-Xaa3-Cys-Gly-Ile-Asp-Asn-Asp-Cys-Cys-Asn-Ala-Cys-Asp-Xaa3- | (SEQ ID NO:249) |
| Ala-Arg-Asn-Ile-Cys-Thr-\hat{ } | |
| Name: Sm6.4 | |
| Species: stercusmuscarum | |
| Cloned: Yes | |
| DNA Sequence: | |
| GGATCCATGAAACTGACGTGTGTGGTGATTGTCGCCGTGCTGCTCCTGACGGCCTGT | (SEQ ID NO:250) |
| CAACTCATCACAGCTGATGACTCCAGAGGTACGCAGGAGCATCGTGCCCTGAGGTC | |
| GAAGACCAAACTCTCCATGTTAACTTTGCGCTGCGTATCTTACGGAAAACCTTGTGG | |
| TATTGACAACGACTGCTGCAATGCATGCGATCCAGCCAGAAATATATGTACGTAGC | |
| TGATCCGGCGTCTGATCTTCCCCCTTCTGTGCTCTATCCTTTTCTGCCTGGGTCCTCC | |
| TTACCTGAGAGTGGTCATGAACCACTCATCACCTACTCCTCTGGAGGCCTCAGAGGA | |
| GTTACAATGAAATAAAAGCCGCATTGCAAAAAAAAAAAAAAAAAAAA | |
| Translation: | |
| MKLTCVVIVAVLLLTACQLITADDSRGTQEHRALRSKTKLSMLTLRCVSYGKLPCGIDND | (SEQ ID NO:251) |
| CCNACDPARNICT | |
| Toxin Sequence: | |
| Cys-Val-Ser-Xaa5-Gly-Lys-Xaa3-Cys-Gly-Ile-Asp-Asn-Asp-Cys-Cys-Asn-Ala-Cys-Asp-Xaa3- | (SEQ ID NO:252) |
| Ala-Arg-Asn-Ile-Cys-Thr-\hat{ } | |
| Name: S6.1 | |
| Species: striatus | |
| Cloned: Yes | |
| DNA Sequence: | |
| ACCAAAACCATCATCAAAATGAAACTGACGTGTGTGGTGATCGTCGCCGTGCTGCT | (SEQ ID NO:253) |
| CCTGACGGCCTGTCAACTCATCACAGCTGATGACTCCAGAGGTACGCAGAAGCATC | |
| GTTCCCTGAGGTCGACCACCAAAGTCTCCAAGGCGACTGACTGCATTGAAGCCGGA | |
| AATTATTGCGGACCTACTGTTATGAAAATCTGCTGCGGCTTTTGCAGTCCATACAGC | |
| AAAATATGTATGAACTATCCCAAAAATTGATCTTCCCCC | |
| Translation: | |
| MKLTCVVIVAVLLLTACQLITADDSRGTQKHRSLRSTTKVSKATDCIEAGNYCGPTVM | (SEQ ID NO:254) |
| KICCGFCSPYSKICMNYPKN | |
| Toxin Sequence: | |
| Ala-Thr-Asp-Cys-Ile-Xaa1-Ala-Gly-Asn-Xaa5-Cys-Gly-Xaa3-Thr-Val-Met-Lys-Ile-Cys-Cys- | (SEQ ID NO:255) |
| Gly-Phe-Cys-Ser-Xaa3-Xaa5-Ser-Lys-Ile-Cys-Met-Asn-Xaa5-Xaa3-Lys-Asn-\hat{ } | |
| Name: S6.2 | |
| Species: striatus | |
| Cloned: Yes | |
| DNA Sequence: | |
| GTCGACTCGCTGCAAGCTTAAAGGACAATCATGTCGTAGGACTATGTATGACTGCTG | (SEQ ID NO:256) |
| CAGCGGTTCTTGCGGCAGGAGAGGTAAATGTGGCTGATCCAGCGCCTGATCTCCCC | |
| CCTTCTGTGCTCTATCCTTTTCTGCCTGGGTCCTCCTTACCTGAGAGTGGTCATGAAC | |
| CACTCATCACCTACTCCTCTGGAGGCCTCAGAGGAGCTACAATGAAATAAAAGCCG | |
| CATTGC | |
| Translation: | |
| STRCKLKGQSCRRTMYDCCSGSCGRRGKCG | (SEQ ID NO:257) |
| Toxin Sequence: | |
| Cys-Lys-Leu-Lys-Gly-Gln-Ser-Cys-Arg-Arg-Thr-Met-Xaa5-Asp-Cys-Cys-Ser-Gly-Ser-Cys- | (SEQ ID NO:258) |
| Gly-Arg-Arg-Gly-Lys-Cys-# | |
| Name: S6.3 | |
| Species: striatus | |
| Cloned: Yes | |
| DNA Sequence: | |
| ACCAAAACCATCATCAAAATGAAACTGACGTGTGTGGTGATCGTCGCCGTGCTGCT | (SEQ ID NO:259) |
| CCTGACGGCCTGTCAACTCATCACAGCTGATGACTCCAGAGGTACGCAGAAGCATC | |
| GTGCCCTGAGGTCGGACACCAAACTCTCCATGTCGACTCGCTGCAAGGCTGCAGGA | |
| AAATCATGCAGTAGGATTGCGTATAACTGCTGCACCGGTTCTTGCAGATCAGGTAA | |
| ATGCGGCTGATCCAGCGCCTGATCTTCCCCCTTCTGTGCTCTATCCTTTCTGCCTGAG | |
| TCCTCTTACCTGAGAGTGGTCATGAACC | |
| Translation: | |
| MKLTCVVIVAVLLLTACQLITADDSRGTQKHRALRSDTKLSMSTRCKAAGKSCSRIAYN | (SEQ ID NO:260) |
| CCTGSCRSGKCG | |
| Toxin Sequence: | |
| Cys-Lys-Ala-Ala-Gly-Lys-Ser-Cys-Ser-Arg-Ile-Ala-Xaa5-Asn-Cys-Cys-Thr-Gly-Ser-Cys-Arg- | (SEQ ID NO:261) |
| Ser-Gly-Lys-Cys-# | |
| Name: S6.6 | |
| Species: striatus | |
| Cloned: Yes | |
| DNA Sequence: | |
| ACCAAAACCATCATCAAAATGAAACTGACGTGTGTGGTGATCGTCGCCGTGCTGCT | (SEQ ID NO:262) |
| CCTGACGGCCTGTCAACTCATCACAGCTGATGACTCCAGAGGTACGCAGGAGCATC | |
| GTGCCCTGAGGTCGGACACCAAACTCTCCATGTTAACTTTGCGCTGCGAATCTTACG | |
| GAAAACCTTGTGGTATTTACAACGACTGCTGCAATGCATGCGATCCAGCCAAAAAG | |
| ACATGTACGTAGCTGATCCGGCGTCTGATCT | |
| Translation: | |
| MKLTCVVIVAVLLLTACQLITADDSRGTQEHRALRSDTKLSMLTLRCESYGKPCGIYND | (SEQ ID NO:263) |
| CCNACDPAKKTCT | |
| Toxin Sequence: | |
| Cys-Xaa1-Ser-Xaa5-Gly-Lys-Xaa3-Cys-Gly-Ile-Xaa5-Asn-Asp-Cys-Cys-Asn-Ala-Cys-Asp- | (SEQ ID NO:264) |
| Xaa3-Ala-Lys-Lys-Thr-Cys-Thr-\hat{ } | |
| Name: w-SVIA | |
| Species: striatus | |
| Cloned: Yes | |
| DNA Sequence: | |
| ACTAGGTCCTCCGGCAGCCCCTGTGGTGTTACTAGTATATGCTGTGGTAGATGCTAT | (SEQ ID NO:265) |
| AGGGGTAAATGTACGTAGCTCATCGGGCGTCTGATCTTCCCCCTTCTGTGCTCCATC | |
| CTTTTCTGCCTGAGTCCTCCTTACCTGAGAGTGGTCGTGAACCACTCATCGCCTACTC | |
| CTCTGGAGGCTTCAGAGGGGCTACACTAAAATAAAAGCTATATTGCAATGAAAAA | |
| A | |
| Translation: | |
| CRSSGSPCGVTSICCGRCYRGKCT | (SEQ ID NO:266) |
| Toxin Sequence: | |
| Cys-Arg-Ser-Ser-Gly-Ser-Xaa3-Cys-Gly-Val-Thr-Ser-Ile-Cys-Cys-Gly-Arg-Cys-Xaa5-Arg- | (SEQ ID NO:267) |
| Gly-Lys-Cys-Thr-# | |
| Name: w-SVIB | |
| Species: striatus | |
| Isolated: Yes | |
| Toxin Sequence: | |
| Cys-Lys-Leu-Lys-Gly-Gln-Ser-Cys-Arg-Lys-Thr-Ser-Xaa5-Asp-Cys-Cys-Ser-Gly-Ser-Cys-Gly- | (SEQ ID NO:268) |
| Arg-Ser-Gly-Lys-Cys-# | |
| Name: Sx6.1 | |
| Species: striolatus | |
| Cloned: Yes | |
| DNA Sequence: | |
| ACCAAAACCATCATCAAAATGAAACTGACGTGTGTGGTGATCGTCGTCTTGCTGCTC | (SEQ ID NO:269) |
| CTCACGACCTGTCGTCTCATCACAGCTGATGACTCCAGAGGTACGCAGAAGCATCG | |
| TTCCCTGAGGTCGACTACTAAAGTCTCCATGTCGACTCGCTGCAAGGGTAAAGGAG | |
| CATCATGTCTTAGGACTGCGTATGACTGCTGCACCGGTTCTTGCAACAGAGGTAGAT | |
| GTGGCTGATCCAGCGTCTGATCTTCCCCCTTCTGTGCTCTATCCTTTTCTGCTTGAGT | |
| CCTCCTTA | |
| Translation: | |
| MKLTCVVIVVLLLLTTCRLITADDSRGTQKHRSLRSTTKVSMSTRCKGKGASCLRTAYD | (SEQ ID NO:270) |
| CCTGSCNRGRCG | |
| Toxin Sequence: | |
| Cys-Lys-Gly-Lys-Gly-Ala-Ser-Cys-Leu-Arg-Thr-Ala-Xaa5-Asp-Cys-Cys-Thr-Gly-Ser-Cys- | (SEQ ID NO:271) |
| Asn-Arg-Gly-Arg-Cys-# | |
| Name: Sx6.2 | |
| Species: striolatus | |
| Cloned: Yes | |
| DNA Sequence: | |
| ACCAAAACCATCATCAAAATGAAACTGACGTGTGTGGTGATCGTCGCCGTTCTGCTG | (SEQ ID NO:272) |
| ACGGCGTGTCAACTCATCACAGCTGAGGACTCCAGAGGTACACAGAAGCATCGTAC | |
| CCTGAGGTCGACCGTCAGACGCTCCAAGTCCGAGTTGACTACGAGATGCAGGCCTT | |
| CAGGATCCAACTGTGGTAATATTAGTATCTGCTGTGGTAGATGCGTTAACAGAAGAT | |
| GTACGTAGCTCATCGGGCGTCTGATCTTTCCCC | |
| Translation: | |
| MKLTCVVIVAVLLTACQLITAEDSRGTQKHRTLRSTVRRSKSELTTRCRPSGSNCGNISI | (SEQ ID NO:273) |
| CCGRCVNRRCT | |
| Toxin Sequence: | |
| Cys-Arg-Xaa3-Ser-Gly-Ser-Asn-Cys-Gly-Asn-Ile-Ser-Ile-Cys-Cys-Gly-Arg-Cys-Val-Asn-Arg- | (SEQ ID NO:274) |
| Arg-Cys-Thr-\hat{ } | |
| Name: Sx6.3 | |
| Species: striolatus | |
| Cloned: Yes | |
| DNA Sequence: | |
| ACCAAAACCATCATCAAAATGAAACTGACGTGTGTGGTGATCGTCGCCGTTCTGTTC | (SEQ ID NO:275) |
| CTGACGGCGTGTCAACTCATCACAGCTGAGGACTCCAGAGGTACACAGAAGCATCG | |
| TTCCCTGAGGTCGACTACCAAAGTCTCCAAGTCGACTAGCTGCATGAAAGCCGGGT | |
| CTTATTGCGTCGCTACTACGAGAATCTGCTGCGGTTATTGCGCTTATTTCGGCAAAA | |
| TATGTATTGACTATCCCAAAAACTGATCTTCCCCCTACTGTGCTCTATCCTTTT | |
| Translation: | |
| MKLTCVVIVAVLFLTACQLITAEDSRGTQKHRSLRSTTKVSKSTSCMKAGSYCVATTRI | (SEQ ID NO:276) |
| CCGYCAYFGKICIDYPKN | |
| Toxin Sequence: | |
| Ser-Thr-Ser-Cys-Met-Lys-Ala-Gly-Ser-Xaa5-Cys-Val-Ala-Thr-Thr-Arg-Ile-Cys-Cys-Gly-Xaa5- | (SEQ ID NO:277) |
| Cys-Ala-Xaa5-Phe-Gly-Lys-Ile-Cys-Ile-Asp-Xaa5-Xaa3-Lys-Asn-\hat{ } | |
| Name: Tx6.15 | |
| Species: textile | |
| Cloned: Yes | |
| DNA Sequence: | |
| GTTGACTCGGTACTGCACGCCTCATGGAGGACATTGTGGTTATCATAATGACTGCTG | (SEQ ID NO:278) |
| CAGTCATCAATGCAATATAAACAGAAATAAATGTGAGTAGCTGATCTGGCATCTGA | |
| TCTGTGCTCGTCCTTACCTGAGAGTGGTCATGAACCACTCATCACCTACTCCTCTGG | |
| AGGC | |
| Translation: | |
| LTRYCTPHGGHCGYHNDCCSHQCNINRNKCE | (SEQ ID NO:279) |
| Toxin Sequence: | |
| Xaa5-Cys-Thr-Xaa3-His-Gly-Gly-His-Cys-Gly-Xaa5-His-Asn-Asp-Cys-Cys-Ser-His-Gln-Cys- | (SEQ ID NO:280) |
| Asn-Ile-Asn-Arg-Asn-Lys-Cys-Xaa1-\hat{ } | |
| Name: w-Tx | |
| Species: textile | |
| Isolated: Yes | |
| Toxin Sequence: | |
| Xaa5-Cys-Thr-Xaa3-Xaa5-Gly-Gly-His-Cys-Gly-Xaa5-His-Asn-Asp-Cys-Cys-Ser-His-Gln- | (SEQ ID NO:281) |
| Cys-Asn-Ile-Asn-Arg-Asn-Lys-Cys-Xaa1-\hat{ } | |
| Name: C. tulipa w2 | |
| Species: tulipa | |
| Cloned: Yes | |
| DNA Sequence: | |
| ACCAAAACCATCATCAAAATGAAACTGACGTGTGTGGTGATCGTCGCCGTGCTGCT | (SEQ ID NO:282) |
| CCTGACGGCCTGTCAGCTCATCACAGCTCTGCACTCCAGAGGTACGCAGAAGCATC | |
| GTGCCCTGGGGCGGACCACCAAACTCACCTTGTCGACTCGCTGCAAATCACCCGGA | |
| TCTCCATGTTCACCGACTAGTTATAATTGCTGCTGGTCTTGCAGTCCATACAGAAAA | |
| AAATGTAGGGGCTAATCCAGCGCCTGATTTTCCCCCTTCTGTGCTCTATTCCTTTCTG | |
| CCTGAGTCCTCCTTACCTGAAAGTGGTCATGAACCACTCATCACCTACTTCTCTGGA | |
| GGCTTCGGAGGAGCTACATTGAAATAAAAGCCGCATTGC | |
| Translation: | |
| MKLTCVVIVAVLLLTACQLITALHSRGTQKHRALGRTTKLTLSTRCKSPGSPCSPTSYNC | (SEQ ID NO:283) |
| CWSCSPYRKKCRG | |
| Toxin Sequence: | |
| Cys-Lys-Ser-Xaa3-Gly-Ser-Xaa3-Cys-Ser-Xaa3-Thr-Ser-Xaa5-Asn-Cys-Cys-Xaa4-Ser-Cys- | (SEQ ID NO:284) |
| Ser-Xaa3-Xaa5-Arg-Lys-Lys-Cys-Arg-# | |
| Name: w-TVIA | |
| Species: tulipa | |
| Cloned: Yes | |
| DNA Sequence: | |
| ACCAAAACCATCATCAAAATGAAACTGACGTGTGTGGTGATCGTCGCCGTGCTGCT | (SEQ ID NO:285) |
| CCTGACGGCCTGTCAGCTCATCACAGCTCTGCACTCCAGAGGTACGCAGAAGCATC | |
| GTGCCCTGGGGTCGACCACCAAACTCACCTTGTCGACTCGCTGCTTGTCACCCGGAT | |
| CTTCATGTTCACCGACTAGTTATAATTGCTGCAGGTCTTGCAATCCATACAGCAGAA | |
| AATGTAGGGGCTAATCCAGCGCCTGATCTTCCCCCTTCTGTGCTCTATTCCTTTCTGC | |
| CTGAGTCCTCCTTACCTGAAAGTGGTCATGAACCACTCATCACCTACTTCTCTGGAG | |
| GCTTCGGAGGAGCTACATTGAAATAAAAGCCGCATTGC | |
| Translation: | |
| MKLTCVVIVAVLLLTACQLITALHSRGTQKHRALGSTTKLTLSTRCLSPGSSCSPTSYNC | (SEQ ID NO:286) |
| CRSCNPYSRKCRG | |
| Toxin Sequence: | |
| Cys-Leu-Ser-Xaa3-Gly-Ser-Ser-Cys-Ser-Xaa3-Thr-Ser-Xaa5-Asn-Cys-Cys-Arg-Ser-Cys-Asn- | (SEQ ID NO:287) |
| Xaa3-Xaa5-Ser-Arg-Lys-Cys-Arg-# | |
| Name: Vi6.1 | |
| Species: viola | |
| Cloned: Yes | |
| DNA Sequence: | |
| ACCAAAACCATCATCAAAATGAAACTGACGTGTGTGGTGATCGTCGCCGTGCTGCT | (SEQ ID NO:288) |
| CCTGACGGCCTGTCAGCTCATTACAGCTGATGACTCCAGAGGTACGCAGTTGCATCG | |
| TGCCCTGAGGAAGGCCACCAAACTCCCCGTGTCGACTCGCTGCATTACTTTAGGAAC | |
| ACGATGTAAGGTTCCGAGTCAATGCTGCAGATCTTCTTGCAAGAACGGTCGTTGTGC | |
| TCCATCCCCTGAAGAATGGTAAATGTGGCTGATCCAGCGCCTGATCTTCCCCCTTCT | |
| GACTGTCTCCGACCTTTTCTGCCTGAGTCCTCCTTACCTGAGAGGTGTCATGAACCA | |
| CTCATCACCTACTCCCCTGGAAGCTTCAGAGGAGCTACATTGAAATAAAAGCCGCA | |
| TTGC | |
| Translation: | |
| MKLTCVVIVAVLLLTACQLITADDSRGTQLHRALRKATKLPVSTRCITLGTRCKVPSQC | (SEQ ID NO:289) |
| CRSSCKNGRCAPSPEEW | |
| Toxin Sequence: | |
| Cys-Ile-Thr-Leu-Gly-Thr-Arg-Cys-Lys-Val-Xaa3-Ser-Gln-Cys-Cys-Arg-Ser-Ser-Cys-Lys-Asn- | (SEQ ID NO:290) |
| Gly-Arg-Cys-Ala-Xaa3-Ser-Xaa3-Xaa1-Xaa1-Xaa4-\hat{ } | |
| Name: Vi6.2 | |
| Species: viola | |
| Cloned: Yes | |
| DNA Sequence: | |
| ACCAAAACCATCATCAAAATGAAACTGACGTGTGTGGTGATCGTCGCCGTGCTGCT | (SEQ ID NO:291) |
| CCTGACGGCCTGTCAGCTCATTATAGCTGGGGACTCCAGAGGTACGCAGTTGCATCG | |
| TGCCCTGAGGAAGGCCACCAAACTCTCCGTGTCGACTCGCTGCAAGAGTAGAGGAT | |
| CATCATGTCGTAGGACTTCGTATGACTGCTGCACGGGTTCTTGCAGAAATGGTAAAT | |
| GTGGCTGATCCAGCGCCTGATCTTCCCCCTTCTGTGCTCCATCCTTTTCTGCCTGAGT | |
| CCTCCTTACCTGAGAGTGGGCATGAACCACTCATCACCTACTCCCTGGAAGCTTCAG | |
| AGGAGCTACATTGAAATAAAAGCCGCATTGC | |
| Translation: | |
| MRLTCVVIVAVLLLTACQLIIAGDSRGTQLHRALRKATKLSVSTRCKSRGSSCRRTSYD | (SEQ ID NO:292) |
| CCTGSCRNGKCG | |
| Toxin Sequence: | |
| Cys-Lys-Ser-Arg-Gly-Ser-Ser-Cys-Arg-Arg-Thr-Ser-Xaa5-Asp-Cys-Cys-Thr-Gly-Ser-Cys-Arg- | (SEQ ID NO:293) |
| Asn-Gly-Lys-Cys-# | |
| Name: Vi6.3 | |
| Species: viola | |
| Cloned: Yes | |
| DNA Sequence: | |
| ACCAAAACCATCATCAAAATGAAACTGACGTGTGTGGCGATCGTCGCCGTGCTGCT | (SEQ ID NO:294) |
| CCTGACGGCCTGTCAGCTCATTACAGCTGAAGACTCCAGAGGTACGCATGAGCATC | |
| TTGCCCTGAAGTCGACCTCCAAAGTCTCCAAGTCGACTAGCTGCATGGAAGCCAGA | |
| TCTTATTGCGGACCTGCTACTACGAAAATCTGCTGCGATTTTTGCAGTCCATTCAGC | |
| GATAGATGTATGAACAATCCCAACAATTGATCTTCCCCCTTGTGTGCTCCATCTTTTC | |
| TGCCTGAGTCCTCCTTACCTGAGAGTGGTCATGAACCACTCATCACCTACTCCTCTG | |
| GAGGCTTCAGAGGAGTTACATTGAAATAAAAGCCGCATGC | |
| Translation: | |
| MKLTCVAIVAVLLLTACQLITAEDSRGTHEHLALKSTSKVSKSTSCMEARSYCGPATTKI | (SEQ ID NO:295) |
| CCDFCSPFSDRCMNNPNN | |
| Toxin Sequence: | |
| Ser-Thr-Ser-Cys-Met-Xaa1-Ala-Arg-Ser-Xaa5-Cys-Gly-Xaa3-Ala-Thr-Thr-Lys-Ile-Cys-Cys- | (SEQ ID NO:296) |
| Asp-Phe-Cys-Ser-Xaa3-Phe-Ser-Asp-Arg-Cys-Met-Asn-Asn-Xaa3-Asn-Asn-\hat{ } | |
| Name: Vi6.4 | |
| Species: viola | |
| Cloned: Yes | |
| DNA Sequence: | |
| ACCAAAACCATCATCAAAATGAAACTGACGTGTGTGGTGATCGTCGCCGTGCTGCT | (SEQ ID NO:297) |
| CCTGACGGCCTGTCAGCTCATTACAGCTGAGGACTCCAGAGGTACGCAGTTGCATC | |
| GTGCCCTGAGGAAGACCACCAAACTCTCCTTGTCGACTCGCTGCAAGGGTCCAGGA | |
| GCCATATGTATAAGGATTGCGTATAACTGCTGCAAGTATTCTTGCGGAAATGGTAAA | |
| TGTGGCTGATCCAGCGCCTGATCTTCCCCCTTGTGTGCTCCATCCTTTTTCTGCCTGA | |
| GTCCTCCTTACCTGAGAGTGGTCATGAACCACTCATCACCTACTCCTCTGGAGGCTT | |
| CAGAGGAGCTACATTGAAATAAAAGCCGCATGC | |
| Translation: | |
| MKLTCVVIVAVLLLTACQLITAEDSRGTQLHRALRKTTKLSLSTRCKGPGAICIRIAYNC | (SEQ ID NO:298) |
| CKYSCGNGKCG | |
| Toxin Sequence: | |
| Cys-Lys-Gly-Xaa3-Gly-Ala-Ile-Cys-Ile-Arg-Ile-Ala-Xaa5-Asn-Cys-Cys-Lys-Xaa5-Ser-Cys- | (SEQ ID NO:299) |
| Gly-Asn-Gly-Lys-Cys-# | |
| Name: Vi6.5 | |
| Species: viola | |
| Cloned: Yes | |
| DNA Sequence: | |
| ACCAAAACCATCATCAAAATGAAACTGACGTGTGTGGTGATCGTCGCCGTGCTGTTC | (SEQ ID NO:300) |
| CTGACGGCCTGTCAATTCATCACAGCTGATGACTCCAGAAGTACGCAGAAGCATCG | |
| TGCCCTGAGGTCGACCACCAAACACTTTATGTTGACTTGGTACTGCACGCCTTATGG | |
| AGGACATTGTGGTTATTATAATGACTGCTGCAGTCATCAATGCAATATAAACAGAA | |
| ATAAATGTGAGTAGCTGATCCGGCATCTGATCTGTGCTCGCCCTAACCTGAGAGTGG | |
| TCATGAACCACTCATCATCTACTCCTCTGGAGGCTTCAGAGGAGCTACATGGAAATA | |
| AAAGCCGCATTGC | |
| Translation: | |
| MKLTCVVIVAVLFLTACQFITADDSRSTQKHRALRSTTKHFMLTWYCTPYGGHCGYYN | (SEQ ID NO:301) |
| DCCSHQCNINRNKCE | |
| Toxin Sequence: | |
| Xaa5-Cys-Thr-Xaa3-Xaa5-Gly-Gly-His-Cys-Gly-Xaa5-Xaa5-Asn-Asp-Cys-Cys-Ser-His-Gln- | (SEQ ID NO:302) |
| Cys-Asn-Ile-Asn-Arg-Asn-Lys-Cys-Xaa1-\hat{ } | |
| Name: Pu6.4 | |
| Species: pulicarius | |
| Cloned: Yes | |
| DNA Sequence: | |
| GGATCCATGAAACTGACGTGCGTGGTGATTATCGCCGTGCTGTTCCTGACGGCCTGT | (SEQ ID NO:303) |
| CAACTCATTACAGCTGAGACTTACTCCAGAGGTAAGCAGATGCACCGTGCTCTGAG | |
| GTCAACTGACAAAAACTCCAAGTTGACCAGGGAATGCACACCTCCAGATGGAGCTT | |
| GTGGTTTACCTACACACTGCTGCGGGTTTTGCGATATGGCAAACAACAGATGTCTGT | |
| AAAGCGTCTGATATTCCCCTTCTGTGCTCTATCCTCTTTGGCCTGAGTCATCCATACC | |
| TGTGCTCGAG | |
| Translation: | |
| MKLTCVVIIAVLFLTACQLITAETYSRGKQMHRALRSTDKNSKLTRECTPPDGACGLPT | (SEQ ID NO:304) |
| HCCGFCDMANNRCL | |
| Toxin Sequence: | |
| Xaa1-Cys-Thr-Xaa3-Xaa3-Asp-Gly-Ala-Cys-Gly-Leu-Xaa3-Thr-His-Cys-Cys-Gly-Phe-Cys- | (SEQ ID NO:305) |
| Asp-Met-Ala-Asn-Arg-Cys-Leu-\hat{ } | |
| Name: Pu6.6 | |
| Species: pulicarius | |
| Cloned: Yes | |
| DNA Sequence: | |
| GGATCCATGAAACTGACGTGCGTGGTGATTATCGCCGTGCTGTTCCTGACGGCCTGT | (SEQ ID NO:306) |
| CAACTCATTACAGCTGAGACTTACTCCAGAGGTAAGCAGATGCACCGTGCTCTGAG | |
| GTCAACTGACAAAAACTCCCAGTTGACCAGGGAATGCACACCTCCAGGTGGAGCTT | |
| GTGGTTTACCTACACACTGCTGCGGGTTTTGCGATATGGCAAACAACAGATGTCTGT | |
| AAAGCGTCTGATATTCCCCTTCTGTGCTCTATCCTCTTTGGCCTGAGTCATCCATACC | |
| TGTGCTCGAG | |
| Translation: | |
| MKLTCVVIIAVLFLTACQLITAETYSRGKQMHRALRSTDKNSQLTRECTPPGGACGLPT | (SEQ ID NO:307) |
| HCCGFCDMANNRCL | |
| Toxin Sequence: | |
| Xaa1-Cys-Thr-Xaa3-Xaa3-Gly-Gly-Ala-Cys-Gly-Leu-Xaa3-Thr-His-Cys-Cys-Gly-Phe-Cys- | (SEQ ID NO:308) |
| Asp-Met-Ala-Asn-Asn-Arg-Cys-Leu-\hat{ } | |
| Name: Ra6.4 | |
| Species: rattus | |
| Cloned: Yes | |
| DNA Sequence: | |
| GGATCCATGAAACTGACGTGTGTGGTGATCATCGCCGTGCTGTTCCTGGCAGCCTGT | (SEQ ID NO:309) |
| CAACCTGTTACAACTGAGACTTTCTCCAGAGGTAAGGAGAAGCGTCGTGCTCTGAG | |
| GTCAACTGACGGCAACTCCCGGTTGACCAGGGCATGCACGCCTGAAGGTGGAGCCT | |
| GTAGTAGTGGGCGTCACTGCTGCGGCTTTTGCGATAACGTGTCCCACACGTGCTATG | |
| GTGAAACACCATCTCTCCACTGATGTTTCCCCTTCTGTGCTCTATCTTCTTTTGCCTG | |
| AGTCATCCATACCTGTGCTCGAG | |
| Translation: | |
| MKLTCVVIIAVLFLAACQPVTTETFSRGKEKRRALRSTDGNSRLTRACTPEGGACSSGR | (SEQ ID NO:310) |
| HCCGFCDNVSHTCYGETPSLH | |
| Toxin Sequence: | |
| Ala-Cys-Thr-Xaa3-Xaa1-Gly-Gly-Ala-Cys-Ser-Ser-Gly-Arg-His-Cys-Cys-Gly-Phe-Cys-Asp- | (SEQ ID NO:311) |
| Asn-Val-Ser-His-Thr-Cys-Xaa5-Gly-Xaa1-Thr-Xaa3-Ser-Leu-His-\hat{ } | |
| Name: Sm6.7 | |
| Species: stercusmuscarum | |
| Cloned: Yes | |
| DNA Sequence: | |
| AGATCCATGAAACTGACGTGCGTGGTGATCGTCGCCGTGCTGCTCCTGACGGCCTGT | (SEQ ID NO:312) |
| CAACTCATCACAGCTGATGACTCCAGAGGTACGCAGGAGCATCGTGCCCTGAGGTC | |
| GGACACCAAACTCCCCATATCGACTCGCTGCAAGGGTAAAGGAGCATCATGTCATA | |
| AGACTATGTATGACTGCTGCAGCGGTTCCTGCACCAGAGGTAGATGTGGCTGATCC | |
| AGCGCCTGATCTTCCCCCTTCTGTGCTCTATCCTTTTCTGCCTGAGTCATCATACCTG | |
| TGCTCGAGCGTTACTAGTGGATCCGAGCTCGGTACCAAGCTTGGCGTAATCATAAA | |
| ANC | |
| Translation: | |
| MKLTCVVIVAVLLLTACQLITADDSRGTQEHRALRSDTKLPISTRCKGKGASCHKTMYD | (SEQ ID NO:313) |
| CCSGSCTRGRCG | |
| Toxin Sequence: | |
| Cys-Lys-Gly-Lys-Gly-Ala-Ser-Cys-His-Lys-Thr-Met-Xaa5-Asp-Cys-Cys-Ser-Gly-Ser-Cys-Thr- | (SEQ ID NO:314) |
| Arg-Gly-Arg-Cys-# | |

Xaa1 = Glu or γ-Carboxy Glu
Xaa2 = Gln or pyroGlu
Xaa3 = Pro or Hydroxy Pro
Xaa4 = Trp or Bromo Trp
Xaa5 = Tyr, ¹²⁵I-Tyr, mono-iodo-Tyr or di-iodo-Tyr or O-sulpho-Tyr or O-Phospho-Tyr
\hat{ } = Free-carboxyl C-term or Amidated C-term, preferably Free-carboxyl
# = Free-carboxyl C-term or Amidated C-term, preferably Amidated

TABLE 2
+HZ,40
Alignment of ω-Conopeptides (SEQ ID NO:)

| | | |
|---|---|---|
| Ar6.10 (F170) | ---QCSANGGSC-TRHFH---CCSLYCNKDSSVCVATSYP\hat{ } | (315) |
| Ar6.2 (F074) | ---TCNTPTEYC-TLHRH---CCSGYCHKTIQACS\hat{ } | (316) |
| Ar6.3 | ---QCTPNGGSC-SRHFH---CCSLYCNKSTGVCIATSYP\hat{ } | (317) |
| Ar6.4 (F009) | ---TCNTPTEYC-TLHQH---CCSGYCHKTIQACS\hat{ } | (318) |
| Ar6.6 (F069) | ---ECTPPGGACGLPT-H---CC-GFCDTANNRCL\hat{ } | (319) |
| Ar6.7 (F073) | ---TCNTPTEYC-TLHQH---CCSGHCHKTIQACA\hat{ } | (320) |
| Ar6.8 (F169) | ---QCSPIGGYC-TLHIH---CCSNHCIKPIGRCVAT\hat{ } | (321) |
| Ar6.9 (F171) | ---QCLPNGGYC-TLHIH---CCSDHCIKPIDRCVAT\hat{ } | (322) |
| Ay6.1 (A653) | ----CKGKGKPCSRISYN---CCTGSCRS--GKC# | (323) |
| Ay6.2 (A654) | ----CMEAGSYCG-STTR--ICC-GFCAYFGKKCIDYPSN\hat{ } | (324) |
| Ay6.3 (J419) | ----CKAKGKPCSRIAYN---CCTGSCRS--GKC# | (325) |
| Ay6.4 | ----CASYGKPCGIDN-D---CCNA-CDPGRNICT\hat{ } | (326) |
| Bu6.1 | -STSCMEAGSYCGPATTK--ICC-DFCSPFSDRCMNNPNN\hat{ } | (327) |
| Bu6.2 | ----CITPGTRCKVPS-Q---CCRGPCKNGR--CTPSPSEW\hat{ } | (328) |
| Bu6.3 | ----CATYGKPCGIQN-D---CC-NTCDPARRTCT\hat{ } | (329) |
| Bu6.4 | ----CKGPGASCIRIAYN---CCKYSCRN---GKC# | (330) |
| Bu6.5 | -STSCMAAGSYCGPATTN--ICC-DFCSPFSDRCMKKPNN\hat{ } | (331) |
| Bu6.6 | ----CKSKGSSCHRTSYD---CCTGSCRN--GRC# | (332) |
| C6.1 | ----CKSTGASCRRTSYD---CCTGSCRS--GRC# | (333) |
| C6.4 | ----CQGRGASCRKTMYN---CCSGSCN--RGSC# | (334) |
| C6.5 | ----CLPAGESCLFSRIR---CC-GTCSSVLKSCVS\hat{ } | (335) |
| C6.6 | ----CQGRGGPCTKAVFN---CCSGSCN--RGRC# | (336) |
| C6.7 | ----CATYGKPCGIQN-D---CC-NTCDPARKTCT\hat{ } | (337) |
| C6.8 | ----CRGRGGPCTKAMFN---CCSGSCN--RGRC# | (338) |
| Ca6.4 (F168) | ---QCSANGGSC-TRHFH---CCSLYCNKDSSVCVATSYP\hat{ } | (339) |
| Cn6.1 | ----CASYGKPCGIDN-D---CC-NTCDPARKTCT\hat{ } | (340) |
| Cn6.2 (I583) | ----CKGTGKPCSRIAYN---CCTGSCRS--GKC# | (341) |
| Cn6.3 | -ATDCIEAGNYCGPTVMK--ICC-GFCSPYSKICMNYPQN\hat{ } | (342) |
| Cn6.4 | ----CKGKGASCTRLMYD---CCHGSCSSSKGRC# | (343) |
| Cn6.5 (I590) | ----CKGKGASCHRTSYD---CCTGSCN--RGKC# | (344) |
| Cn6.6 (I584) | ----CASYGKPCGIYN-D---CC-NTCDPARKTCT\hat{ } | (345) |
| Cn6.7 (J409) | ----CKGTGKPCSRVAYN---CCTGSCRS--GKC# | (346) |
| Cn6.8 (J407) | -STSCMKAGSYCR-STTR--TCC-GYCAYFGKFCIDFPSN\hat{ } | (347) |
| Cr6.1 | ----CKGKGASCRKTMYN---CCSGSCSN--GRC# | (348) |
| Cr6.2 | -STSCMEAGSYCR-STTR--TCC-GYCSYFSKKCIDFPSN\hat{ } | (349) |
| Cr6.3 | ----CKSKGAKCSRLMYD---CCSGSCSRYSGRC# | (350) |
| Cr6.4 | -STGCMKAGSYCR-STTR--TCC-GYCAYFGKKCIDYPSN\hat{ } | (351) |
| Da6.8 | ---SCTPPGGPCGYYN-D---CCSHQCNISRNKCE\hat{ } | (352) |
| Di6.1 | ----CEDOGEOCGSDH-S---CCGGSCN--HNVCA\hat{ } | (353) |
| E6.2 | ---PCKPKGRKCFPHQKD---CCNKTCT--RSKCP\hat{ } | (354) |
| E6.3 | ---ACWSSGTPCGTDS-L---CCGG-CNVSKSKCN\hat{ } | (355) |
| G6.1 (J420) | ----CKSPGSSCSPTSYN---CCR-SCNPYAKRCY# | (356) |
| G6.2 (J423) | ----CKSPGTPCSRGMRD---CCT-PCLLYSNKC-R--RY\hat{ } | (357) |
| J410 | ----CLSPGSRCHKTMRN---CCT-SCSSYKGKCRP--RK\hat{ } | (358) |
| J411 | ----CKPPGRKCLNRKNE---CCSKFCNEHLHMC# | (359) |
| J413 | ----CKPPRRKCLKIKDK---CC-NFCNTHLNMC# | (360) |
| J414 | ----CAGPGTIC--PNRV---CC-GYCSKRTHLCHS---RT# | (361) |
| La6.1 | ---KCWPSGSYCRANS-K---CCSG-CDRNRNKCY\hat{ } | (362) |
| La6.2 | ----CLPPGSYCK-ATTE--VCCS-SCLQFAQIC-----S# | (363) |
| L6.1 | ----CKSPGSPCSVTSYN---CCT-FCSSYTKKCRA--SL\hat{ } | (364) |
| L6.2 | ----CAGPGAIC--PNRV---CC-GYCSKRTHLCHS---RT# | (365) |
| L6.3 | ---ACWSSGTPCGTDS-L---CCGG-CNVSKSKCN\hat{ } | (366) |
| L6.4 | ---KCWSPGTYCRAHS-K---CCRG-CDQNRNKCY\hat{ } | (367) |
| La6.3 | ----CKSPGSSCSVSMRN---CCT-SCNSRTKKCTR--R# | (368) |
| La6.4 | ---TCWPSGTACGIDS-N---CCSG-CNVSRSKCN\hat{ } | (369) |
| La6.5 | ---KCWPSGSYCRANS-K---CCSG-CDRNRSKCN\hat{ } | (370) |
| Lp6.1 (JG4) | SLFECAPSGGRCGFLK-S---CCEGYCDGESTSCVSGPYSI\hat{ } | (371) |
| Lp6.2 (JG5) | WPLDCTAPSQPCGYFP-R---CCG-HCDV-RRVCTS# | (372) |
| Lp6.3 (JG7) | ----CMSPGGICGDFG-D---CCE-ICNV-YGICVSDLPGI\hat{ } | (373) |
| Lp6.4 (JG15) | ---YCAPPGGACGFFD-H---CC-GYCETFYNTC-R\hat{ } | (374) |
| M6.1 | ----CKGTGKPCSRIAYN---CCTGSCRS-GKC# | (375) |
| M6.2 | ----CASYGKPCGIYN-D---CC-NTCDPARKTCT\hat{ } | (376) |
| Mi6.1 (F157) | ----CNDRGGGC-SQHPH---CCGGTCNKLIGVCL\hat{ } | (377) |
| Mn6.1 | ----CKSTGKSCSRIAYN---CCTGSCRS--GKC# | (378) |
| Mn6.2 | ----CKGKGSSCSRTMYN---CCTGSCN--RGKC# | (379) |
| O6.1 | -SPPCMKGGSSCR-GTTG--VCC-GFCSDFGYKCRDYPQN\hat{ } | (380) |
| O6.2 | ----CLPDGTSCLFSRIR---CC-GTCSSILKSCVS\hat{ } | (381) |
| P6.1 | ---OCKTOGRKCFOHQKD---CCGRACI--ITICP\hat{ } | (382) |
| P6.2 | ---SCKLOGAYCNAXDYD---CCLR-CKV-GGTC# | (383) |
| P6.3 | ---PCKKTGRKCFPHQKD---CCGRACI--ITICP\hat{ } | (384) |
| Pu6.2 (JG28) | ---QCSPNGGSC-SRHFH---CCSLYCNKNTGVCIAT\hat{ } | (385) |
| Pu6.4 (AA678) | ---ECTPPDGACGLPT-H---CC-GFCDMANNRCL\hat{ } | (386) |
| Pu6.6 (AAG81) | ---ECTPPGGACGLPT-H---CC-GFCDMANNRCL\hat{ } | (387) |
| R6.1 | --HGCKPLKRRCFNDKE----CCSKFCNSVRKQC# | (388) |
| R6.2 | --RGCKPLKRRCFNDKE----CCSKFCNSVRNQC# | (389) |
| Ra6.1 (F206) | ----CNARNDGC-SQHSQ---CCSGSCNKTAGVCL\hat{ } | (390) |
| Ra6.2 (F205) | ----CNARNSGC-SQHPQ---CCSGSCNKTAGVCL\hat{ } | (392) |
| Ra6.3 (F207) | ----CNARNSGC-SQHPQ---CCSGSCNKTLGVCL\hat{ } | (393) |
| Ra6.4 (AA688) | ---ACTPEGGACSSGR-H---CC-GFCDNVSHTCYGETPSLH\hat{ } | (394) |
| S6.1 | -ATDCIEAGNYCGPTVMK--ICC-GFCSPYSKICMNYPKN\hat{ } | (395) |
| S6.2 | ----CKLKGQSCRRTMYD---CCSGSCGR-RGKC# | (396) |
| S6.3 | ----CKAAGKSCSRIAYN---CCTGSCRS--GKC# | (397) |
| S6.6 | ----CESYGKPCGIYN-D---CC-NACDPAKKTCT\hat{ } | (398) |
| Sm6.1 (J428) | ----CKSKGAKCSRLMYD---CCSGSCSGYTGRC# | (399) |
| Sm6.2 | -TTSCMQAGSYCG-STTR--ICC-GYCAYFGKKCIDYPSN\hat{ } | (400) |
| Sm6.3 (J429) | ----CASYGKPCGIDN-D---CC-NACDPARNICT\hat{ } | (401) |
| Sm6.4 (J431) | ----CVSYGKPCGIDN-D---CC-NACDPARNICT\hat{ } | (402) |
| Sm6.7 (AAG89) | ----CKGKGASCHKTMYD---CCSGSCTRG--RC# | (403) |
| Sx6.1 | ----CKGKGASCLRTAYD---CCTGSCN--RGRC# | (404) |
| Sx6.2 | ----CRPSGSNCGNIS-I---CCGR-CVN--RRCT\hat{ } | (405) |
| Sx6.3 | -STSCMKAGSYCV-ATTR--ICC-GYCAYFGKICIDYPKN\hat{ } | (406) |
| Tx6.15 | ---YCTPHGGHC-GYHND---CCSHQCNINRNKCE\hat{ } | (407) |
| Vi6.1 | ----CITLGTRCKVPS-Q---CCRSSCKN--GRCAPSPEEW\hat{ } | (408) |
| Vi6.2 | ----CKSRGSSCRRTSYD---CCTGSCRN--GKC# | (409) |
| Vi6.3 | -STSCMEARSYCGPATTK--ICC-DFCSPFSDRCMNNPNN\hat{ } | (410) |
| Vi6.4 | ----CKGPGAICIRIAYN---CCKYSCGN--GKC# | (411) |
| Vi6.5 | ---YCTPYGGHCGYYN-D---CCSHQCNINRNKCE\hat{ } | (412) |
| ω-Tx | ----CTPYGGHCGYNH-D---CCSHQCNINRNKCE\hat{ } | (413) |
| C. tulipa ω2 | ----CKSWGSOCSOTSTN---CCW-SCSPYRKKC-R# | (414) |

### Example 3

### In vivo Activity of ω-Conopeptide

### Frings Audiogenic Seizure Susceptible Mice

In vivo anticonvulsant activity of ω-conopeptides is analyzed in Frings audiogenic seizure susceptible mice as described by White et al. (1992). The ω-conopeptides are found to have anticonvulsant activity in this assay.

### Example 4

### In vivo Activity of ω-Conopeotides in CF No. 1 Mice

In vivo anticonvulsant activity of o)conopeptides is analyzed in CF No. 1 mice as described by White et al. (1995), using the maximal electroshock, subcutaneous pentylenetetrazole (Metrazol) seizure threshold and threshold tonic extension test. ω-Conopeptides are found to have anticonvulsant activity.

### Example 5

### In Vivo Activity of ω-Conopeptides in

### Pentylenetetrazole-Induced Threshold Seizure Model

The in vivo activity of ω-conopeptides is analyzed using timed intravenous infusion of pentylenetetrazole (White et al., 1995). At time to peak effect, the convulsant solution (0.5% pentylenetetrazole in 0.9% saline containing 10 U.S.P. units/ml heparin sodium) is infused into the tail vein at a constant rate of 0.34 ml/min. The time in seconds from the start of the infusion to the appearance of the first twitch and the onset of clonus is recorded for each drug treated or control animal. The times to each endpoint are converted to mg/kg of pentylenetetrazole for each mouse, and mean and standard error of the mean are calculated. It is found that ω-conopeptides elevate the i.v. pentylenetetrazole seizure threshold.

### Example 6

### In vivo Activity of ω-Conopeptides in Pain Models

The anti-pain activity of ω-conopeptides is shown in several animal models.

These models include the nerve injury model (Chaplan, et al., 1997), the nocioceptive response to s.c. formalin injection in rats (Codene, 1993) and an NMDA-induced persistent pain model (Liu, et al., 1997). In each of these models it is seen that the ω-conopeptides and ω-conopeptides derivatives have analgesic properties.

More specifically, this study evaluates the effect of intrathecal administration of ω-conopeptides in mice models of nocioceptive and neuropathic pain. For nocioceptive pain, the effect of the ω-conopeptides is studied in two different tests of inflammatory pain. The first is the formalin test, ideal because it produces a relatively short-lived, but reliable pain behavior that is readily quantified. There are two phases of pain behavior, the second of which is presumed to result largely from formalin-evoked inflammation of the hind paw. An ω-conopeptide is administered 10 minutes prior to injection of formalin. The number of flinches and/or the duration of licking produced by the injection is monitored. Since the first phase is presumed to be due to direct activation of primary afferents, and thus less dependent on long term changes in the spinal cord, ω-conopeptides are presumed to have greatest effect on the magnitude of pain behavior in the second phase.

The mechanical and thermal thresholds in animals that received an injection of complete Freund's adjuvant into the hind paw are also studied. This produces a localized inflammation including swelling of the hind paw and a profound decrease in mechanical and thermal thresholds, that are detected within 24 hours after injection. The changes in thresholds in rats that receive ω-conopeptides are compared with those of rats that receive vehicle intrathecal injections.

An important issue is whether the drugs are effective when administered after the pain model has been established, or whether they are effective only if used as a pretreatment. Clearly, the clinical need is for drugs that are effective after the pain has developed. To address this issue, animals are studied in which ω-conopeptides are administered repeatedly, after the inflammation (CFA) or nerve injury has been established. In these experiments, an ω-conopeptide is injected daily by the intrathecal (i.t.) route. The mechanical and thermal thresholds (measured, respectively, with von Frey hairs in freely moving animals and with the Hargreave's test, also in freely moving animals) are repeated for a 2 to 4 week period after the injury is induced and the changes in pain measured monitored over time.

### Example 7

### Effect of -Conotoxins in a Pain Model

Analgesic activity of ω-conotoxins is also tested in pain models as follows.

Persistent pain (formalin test). Intrathecal (it) drug injections are performed as described by Hylden and Wilcox (1980). An ω-conopeptide or vehicle is administered in a volume of 5 1. Fifteen minutes after the i.t. injection, the right hindpaw is injected with 20 l of 5% formalin. Animals are placed in clear plexiglass cylinders backed by mirrors to facilitate observation. Animals are closely observed for 2 minutes per 5 minute period, and the amount of time the animal spent licking the injected paw is recorded in this manner for a total of 45-50 minutes. Results are expressed as licking time in seconds per five minutes. At the end of the experiment, all animals are placed on an accelerating rotorod and the latency to first fall was recorded. ω-Conopeptides are found to be active in this model which is predictive of efficacy for treating neuropathic pain.

Acute pain (tail-flick). An ω-conopeptide or saline is administered intrathecally (i.t.) according to the method of Hylden and Wilcox (1980) in a constant volume of 5 μl. Mice are gently wrapped in a towel with the tail exposed. At various time-points following the i.t. injection, the tail is dipped in a water bath maintained at 54 C. and the time to a vigorous tail withdrawal is recorded. If there is no withdrawal by 8 seconds, the tail is removed to avoid tissue damage.

Neuropathic pain. The partial sciatic nerve ligation model is used to assess the efficacy of Marl in neuropathic pain. Nerve injury is produced according to the methods of Malmberg and Basbaum (1998). Animals are anesthetized with a ketamine/xylazine solution, the sciatic nerve is exposed and tightly ligated with 8-0 silk suture around ⅓ to ½ of the nerve. In sham-operated mice the nerve is exposed, but not ligated. Animals are allowed to recover for at least 1 week before testing is performed. On the testing day, mice are placed in plexiglass cylinders on a wire mesh frame and allowed to habituate for at least 60 minutes. Mechanical allodynia is assessed with calibrated von Frey filaments using the up-down method as described by Chaplan et al. (1994), and the 50% withdrawal threshold is calculated. Animals that did not respond to any of the filaments in the series are assigned a maximal value of 3.6 grams, which is the filament that typically lifted the hindlimb without bending, and corresponds to approximately 1/10 the animal's body weight.

The data obtained demonstrate that ω-conopeptides have potent analgesic properties in three commonly used models of pain: acute, persistent/inflammatory and neuropathic pain models.

### Example 8

### Calcium-Channel Antagonist Activity

Inhibition of Ionic Currents

Ionic currents through calcium channels are examined in cells that are voltage-clamped by a single patch-clamp electrode. These whole-cell patch-clamp studies are performed mainly on N1E115 mouse neuroblastoma cells, although a variety of cell types, including human neuroblastoma cell line IMR-32, are also examined.

Most measurements are obtained using a bath saline that allowed examination of the calcium currents in the absence of other ionic currents. These solutions contained 80 mM NMDG (as a sodium replacement), 30 mM TEACl (to block potassium currents), 10 mM BaCl₂ (as a charge-carrier through the calcium channels), and 10 mM HEPES at pH 7.3. Some solutions also contained 2 mM quinidine (to block potassium currents) and 3 μM tetrodotoxin (to block sodium currents). Normal bath saline is (mnM): 140 NaCl, 10 glucose, 3 KCl, 2 CaCl₂, 1 MgCl₂, 10 mM HEPES pH 7.3. Intracellular solutions contained (mM): 150 CsCl, 0.5 CaCl₂, 5 EGTA, 5 MgCl₂, 2 K₂ATP at pH 7.3-7.4. Bath saline and all internal solutions are filtered before use.

Pipets are made from Coming 7052 glass (Garner Glass Company, Claremont, Calif. 91711), coated with Sylgard (Dow Coming, Midland, Mich. 48640) and fire-polished before use. Bubble numbers are typically 5 to 6, with pipet resistances typically 2-5 MOhms.

Corning 8161, Kimble, and other glasses are also used without noticeable effect on the calcium currents observed.

Recordings are carried out at room temperature with an Axopatch 1-C amplifier (Axon Instruments, Foster City, Calif. 94404) and analyzed with pCLAMP software (Axon Instruments). Data are filtered at 1000 Hz for a typical sampling rate of 0.1 kHz; in all cases data are filtered at a frequency at most ⅕ of the sampling rate to avoid biasing. Data are collected on-line by the software. Analysis is performed on-screen with print-out via a Hewlett-Packard LaserJet Printer (Hewlett-Packard, Palo Alto, Calif. 94306).

The typical experiment is conducted as follows: after seal formation followed by series resistance compensation and capacitative transient cancellation, a voltage clamp protocol is performed wherein the cell potential is stepped from the holding potential (typically −100 mV) to test potentials that ranged from −60 mV to +20 mV in 10 mV increments. The cell is held at the holding potential for 5 seconds between pulses. Protocols starting from other holding potentials usually covered the same range of test potentials. ω-Conopeptides are found to have calcium channel blocking activity in such cell lines.

It will be appreciated that the methods and compositions of the instant invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as restrictive.

### BIBLIOGRAPHY

Abiko, H. et al. (1986). Brain Res. 38:328-335.

Aldrete, J. A. et al. (1979). Crit. Care Med. 7:466-470.

Barnay, G. et al. (2000). J. Med. Chem.

Bitan, G. et al. (1997). J. Peptide Res. 49:421-426.

Bodansky et al. (1966). Chem. Ind. 38:1597-98.

Cartier, G. E. et al. (1996). J. Biol. Chem. 271:7522-7528.

Chandler, P. et al. (1993). J Biol. Chem. 268:17173-17178.

Chaplan S. R. (1997). J Pharmacol. Exp. Ther. 280:829-838.

Clark, C. et al. (1981). Toxicon 19:691-699.

Codere, T. J. (1993). Eur. J. Neurosci. 5:390-393.

Cruz, L. J. at al. (1976). Verliger 18:302-308.

Ettinger, L. J. et al. (1978). Cancer 41:1270-1273.

Hammerland et al. (1992). Eur. J. Pharmacol. 226:239-244.

Heading, C. (1999). Curr. Opin. CPNS Invest. Drugs 1:153-166

Horiki, K. et al. (1978). Chemistry Letters 165-68.

Hubry, V. et al. (1994). Reactive Polymers 22:231-241.

Hylden, J. L. K.and Wilcox, G. (1980). Eur. J. Pharmacol. 67:313-316.

Kaiser et al. (1970). Anal. Biochem. 34:595.

Kapoor (1970). J. Pharm. Sci. 59:1-27.

Kornreich, W. D. et al. (1986). U.S. Pat. No. 4,569,967.

Luer, M. S. & Hatton, J. (1993). Annals Pharmcotherapy 27:912-921.

Liu, H. et al. (1997). Nature 386:721-724.

Martinez, J. S. et al. (1995). Biochem. 34:14519-14526.

McIntosh, J. M. et al. (1998). Methods Enzymol. 294:605-624.

The Merck Manual of Diagnosis and Therapy, 16 Ed., Berkow, R. et al., eds., Merck Research Laboratories, Rahway, N.J., pp. 1436-1445 (1992).

Methoden der Organischen Chemie (Houben-Weyl): Synthese von Peptiden, E. Wunsch (Ed.), Georg Thieme Verlag, Stuttgart, Ger. (1974).

Nehlig, A. et al. (1990). Effects of phenobarbital in the developing rat brain. In Neonatal Seizures, Wasterlain, C. G. and Vertt, P. (eds.), Raven Press, New York, pp. 285-194.

Nishiuchi, Y. et al. (1993). Int. J. Pept. Protein Res. 42:533-538.

Olivera, B. M. et al. (1984). U.S. Pat. No. 4,447,356.

Olivera, B. M. et al. (1985). Science 230:1338-1343.

Olivera, B. M. et al. (1990). Science 249:257-263.

Olivera, B. M. et al. (1996). U.S. Pat. No. 5,514,774.

Ornstein, et al. (1993). Biorganic Medicinal Chemistry Letters 3:43-48.

Rall T. W. and Schleifer, L. S. in Goodman and Gilman's The Pharmacological Basis of Therapeutics, Seventh Ed., Gilman, A.G. et al., eds., Macmillan Publishing Co., New York, pp. 446-472 (1985).

Remington's Pharmaceutical Sciences, 18th Ed. (1990, Mack Publishing Co., Easton, Pa.).

Rivier, J. R. et al. (1978). Biopolymers 17:1927-38.

Rivier, J. R. et al. (1987). Biochem. 26:8508-8512.

Sambrook, J. et al. (1989). Molecular Cloning: A Laboratory Manual, 2nd Ed., Cold Spring Harbor Laboratory, Cold Spring Harbor, N.Y.

Shon, K. -J. et al. (1994). Biochemistry 33:11420-11425.

Stewart and Young, Solid-Phase Peptide Synthesis, Freeman & Co., San Francisco, Calif. (1969).

Vale et al. (1978). U.S. Pat. No. 4,105,603.

Troupin, A. S. et al. (1986). MK-801. In New Anticonvulsant Drugs, Current Problems in Epilepsy 4, Meldrum, B. S. and Porter, R. J. (eds.), John Libbey, London, pp. 191-202.

Van de Steen, P. et al. (1998). Critical Rev. in Biochem. and Mol. Biol. 33:151-208.

White, H. S., et al. (1992). Epilepsy Res. 12:217-226.

White, H. S., et al. (1995). Experimental Selection, Quantification, and Evaluation of Antiepilep-tic Drugs. In Antiepileptic Drugs, 4th Ed., Levy, R.H., eds., Raven Press, N.Y., pp. 99-110.

Wong, E. H. P. et al. (1986). Proc. Natl. Acad. Sci. USA 83:7104-7108.

Zhou L. M., et al. (1996). J. Neurochem. 66:620-628.

Zimm, S. et al. (1984). Cancer Res. 44:1698-1701.

U.S. Pat. No. 3,842,067.

U.S. Pat. No. 3,862,925.

U.S. Pat. No. 3,972,859.

U.S. Pat. No. 5,514,774.

U.S. Pat. No. 5,550,050.

U.S. Pat. No. 5,591,821.

U.S. Pat. No. 5,719,264.

U.S. Pat. No. 5,844,077 (1998).

Published PCT Application WO 92/19195.

Published PCT Application WO 94/25503.

Published PCT Application WO 95/01203.

Published PCT Application WO 95/05452.

Published PCT Application WO 96/02286.

Published PCT Application WO 96/02646.

Published PCT Application WO 96/40871.

Published PCT Application WO 96/40959.

Published PCT Application WO 97/12635.

Published PCT Application WO 98/03189.

Published PCT Application WO 00/23092.

What is claimed is:

1. An isolated peptide selected from the group consisiting of:
(a) a peptide set forth in Table 2
(b) a derivative of the peptide in (a); and
(c) a propeptide set forth in Table 1 .

2. The isolated peptide of claim 1, wherein Xaa₁ is Lys, Xaa2 is Tyr, Xaa3 is Glu and Xaa₅ is Trp.

3. An isolated nucleic acid encoding an ω-conopeptide propeptide having an amino acid sequence set forth in Table 1.

4. The isolated nucleic acid of claim 3, wherein the nucleic acid comprises a nucleotide sequence set forth in Table 1.

5. An isolated conopeptide propeptide having an amino acid sequence set forth in Table 1.

6. A method for treating or preventing disorders associated with voltage gated ion channel disorders in which comprises administering to a patient in need thereof a therapeutically effective amount of an active agent selected from the group consisting of peptides (a) and (b) of claim 1 or a pharmaceutically acceptible salt thereof.

7. The method of claim 6, wherein said disorder is a neurologic disorder.

8. The method of claim 7, wherein said neurologic disorder is a seizure.

9. The method of claim 8, wherein said seizure is seizure is associated with epilepsy.

10. The method of claim 7, wherein said neurologic disorder is a neurotoxic injury associated with conditions of hypoxia, anoxia or ischemia.

11. The method of claim 10, wherein said neurotoxic injury is associated with stroke, cerebrovascular accident, brain or spinal cord trauma, myocardial infarct, physical trauma, drownings, suffocation, perinatal asphyxia, or hypoglycemic events.

12. The method of claim 6, wherein said disorder is pain.

13. The method of claim 12, wherein said pain is migraine, acute pain, persistent pain, neuropathic pain or nociceptive pain.

14. The method of claim 6, wherein said disorder is inflammation.

15. The method of claim 6, wherein said disorder is a cardiovascular disorder.


SEQUENCE LISTING (413 sequences)

SEQ ID NO: 1 - DNA, 318 bp, Unknown
  unknown Conus species
ggatccatgaaactgacgtgcatggtgatcgtcgccgtgctgctcctgacggcctgtcaactcatcacagctgatgactccagaggtacgcagaagcatcatgccctgaggtcgaccaccaatttctccacgttgactcgtcgctgcctttctcccggatcacgatgtcataagacaatgcgtaactgctgcacttcatgctcttcatacaaagggaaatgtcggcctcgaaaatgaaccactcatcacctactcctctggaggcctcagaggaattacattgaaataaaagccgcattacaaaaaaaaaaaaaaaaa

SEQ ID NO: 2 - protein, 76 aa, Unknown
  unknown Conus species
MKLTCMVIVAVLLLTACQLITADDSRGTQKHHALRSTTNFSTLTRRCLSPGSRCHKTMRNCCTSCSSYKGKCRPRK

SEQ ID NO: 3 - protein, 30 aa, Unknown
  unknown Conus species
CLSXGSRCHKTMRNCCTSCSSXKGKCRXRK

SEQ ID NO: 4 - DNA, 283 bp, Unknown
  unknown Conus species
ggatccatgaaactgacgtgcgtggtgatcgtcgccgtgctgctcctgacggtctgtcaactcatcacagctgatgactccagaggtacgcagaagcatcatgccctgaggtcgaccaccaatttctccacgtcgactcgtcgctgcaaacctcccggaagaaaatgtctgaatagaaagaatgaatgctgcagcaagttttgcaatgaacacctacatatgtgtggataaatggctaaaaactgaataaaagccgcattgcaaaaaaaaaaaaaaaaaaaaa

SEQ ID NO: 5 - protein, 74 aa, Unknown
  unknown Conus species
MKLTCVVIVAVLLLTVCQLITADDSRGTQKHHALRSTTNFSTSTRRCKPPGRKCLNRKNECCSKFCNEHLHMCG

SEQ ID NO: 6 - protein, 27 aa, Unknown
  unknown Conus species
CKXXGRKCLNRKNXCCSKFCNXHLHMC

SEQ ID NO: 7 - DNA, 275 bp, Unknown
  unknown Conus species
ggatccatgaaactgacgtgcgtggtgatcgtcgccgtgctgctcctgacggcctgtcaactcgtcacagctgatggctccagaggtatgcagaagcattatgccctgaggtcgaccaccaatctctccatatcgtctcgctgcaaacctcccagaagaaaatgtctgaagattaaggataaatgctgcaacttttgcaatacacacctaaatatgtgtggataaatggctaaaaactgaataaaagccgcattgcaaaaaaaaaaaaaaaaaaa

SEQ ID NO: 8 - protein, 72 aa, Unknown
  unknown Conus species
MKLTCVVIVAVLLLTACQLVTADGSRGMQKHYALRSTTNLSISSRCKPPRRKCLKIKDKCCNFCNTHLNMCG

SEQ ID NO: 9 - protein, 26 aa, Unknown
  unknown Conus species
CKXXRRKCLKIKDKCCNFCNTHLNMC

SEQ ID NO: 10 - DNA, 377 bp, Unknown
  unknown Conus species
ggatccatgaaactgacgtgtgtggtgatcgtcgccgtgctgctcctgatggcctgtcaactcgtcacagctgatggctccagaggtatgcacaagcattatgccctgaggtcgaccaccaaactctccatgtcgactcgctgcgcaggtccaggaacaatttgtcctaatagggtatgctgcggttattgcagtaaaagaacacatctatgtcattcgcgaactggctgatcttcccccttctgcgctccatccttttctgcctgagtcctccatacctgagaatggtcatgaaccactcaacacctactcctctggagggcctcagaagagctacattgaaataaaagccgcattacaaaaaaaaaaaaaaaaaa

SEQ ID NO: 11 - protein, 74 aa, Unknown
  unknown Conus species
MKLTCVVIVAVLLLMACQLVTADGSRGMHKHYALRSTTKLSMSTRCAGPGTICPNRVCCGYCSKRTHLCHSRTG

SEQ ID NO: 12 - protein, 28 aa, Unknown
  unknown Conus species
CAGXGTICXNRVCCGXCSKRTHLCHSRT

SEQ ID NO: 13 - DNA, 323 bp, Conus arenatus
ggatccatgaaactgacgtgcatggtgatcatcgccgtgctgttcctgacggcctgtcaactcattacaggtgagcagaaggaccatgctctgaggtcaactgacaaaaactccaagttgactaggcagtgctcggctaacggtggatcttgtactcgtcattttcactgctgcagcctctattgcaataaagattccagtgtatgtgtggcaacctcatacccgtgagtggccatgaacccctcaataccctctcctctggaggcttcagaggaactgcattgaaataaaaccgcattgcaataaaaaaaaaaaaaaaaaaa

SEQ ID NO: 14 - protein, 73 aa, Conus arenatus
MKLTCMVIIAVLFLTACQLITGEQKDHALRSTDKNSKLTRQCSANGGSCTRHFHCCSLYCNKDSSVCVATSYP

SEQ ID NO: 15 - protein, 33 aa, Conus arenatus
  PEPTIDE (1)..(33): Xaa at residue 1 is Gln or pyro-Glu; Xaa at residue 33 is Pro or Hyp; Xaa at residue 19 and 32 is Tyr, 125I-Tyr, mono-iodo-Tyr, di-iodo-Tyr, O-sulpho-Tyr or O-phospho-Tyr
XCSANGGSCTRHFHCCSLXCNKDSSVCVATSXX

SEQ ID NO: 16 - DNA, 326 bp, Conus arenatus
accaaaaccatcatcaaaatgaaactgacgtgcgtgttgattatcgccgtgctgttcctgacggcctgtcaactcattacagctgagacttactccagaggtgagcagaagcaccatgctctgaggtcaactgacagaaactccaagttgaccaggacatgcaacactcccactgaatattgtactttgcatcgacactgctgcagcggctactgccataaaacaatccaggcatgttcataataccggtgagtggtcatgaaccactcaataccctctcctctggaggcttcagaggaactgcattgaaataaaagccgcattgc

SEQ ID NO: 17 - protein, 74 aa, Conus arenatus
MKLTCVLIIAVLFLTACQLITAETYSRGEQKHHALRSTDRNSKLTRTCNTPTEYCTLHRHCCSGYCHKTIQACS

SEQ ID NO: 18 - protein, 28 aa, Conus arenatus
  PEPTIDE (1)..(28): Xaa at residue 7 is Glu or gamma-carboxy Glu; Xaa at residue 5 is Pro or Hyp; Xaa at residue 8 and 19 is Tyr, 125I-Tyr, mono-iodo-Tyr, di-iodo-Tyr, O-sulpho-Tyr or O-phospho-Tyr
TCNTXTXXCTLHRHCCSGXCHKTIQACS

SEQ ID NO: 19 - DNA, 332 bp, Conus arenatus
accaaaaccatcatcaaaatgaaactgacgtgcgtgttgatcatcgccgtgctgttcctgacggcctgtcaactcattacagctgagacttactccagaggtgagcagatgcaccgtgctctgaggtcaactgacaaaaactccaagttgactaggcagtgcacgcctaacggtggatcttgttctcgtcattttcactgctgcagcctctattgcaataaaagtactggcgtatgtattgcaacctcatacccgtgagtggtcatgaaccactcaataccctctcctctggaggcttcagaggaactgcattgaaataaaagccgcattgc

SEQ ID NO: 20 - protein, 79 aa, Conus arenatus
MKLTCVLIIAVLFLTACQLITAETYSRGEQMHRALRSTDKNSKLTRQCTPNGGSCSRHFHCCSLYCNKSTGVCIATSYP

SEQ ID NO: 21 - protein, 33 aa, Conus arenatus
  PEPTIDE (1)..(33): Xaa at residue 1 is Gln or pyro-Glu; Xaa at residue 4 and 33 is Pro or Hyp; Xaa at residue 19 and 32 is Tyr, 125I-Tyr, mono-iodo-Tyr, di-iodo-Tyr, O-sulpho-Tyr or O-phospho-Tyr
XCTXNGGSCSRHFHCCSLXCNKSTGVCIATSXX

SEQ ID NO: 22 - DNA, 332 bp, Conus arenatus
ggatccatgaaactgacgtgcatggtgattatcgccgtgctgttcctgacggcctgtcaactcattacagctgagacttactccagaggtgagcagaagcaccatgctctgaggtcaactgacaaaaactccaagttgaccaggacatgcaacactcccaccgaatattgtactttgcatcaacactgctgcagcggctactgccataaaacaatccaggcatgttcataataccggtgagtggtcatgaaccactcaataccctctcctctggaggcttcagaggaactgcattgaaataaaaccgcattacaaaaaaaaaaaaaaaaaaa

SEQ ID NO: 23 - protein, 74 aa, Conus arenatus
MKLTCMVIIAVLFLTACQLITAETYSRGEQKHHALRSTDKNSKLTRTCNTPTEYCTLHQHCCSGYCHKTIQACS

SEQ ID NO: 24 - protein, 28 aa, Conus arenatus
  PEPTIDE (1)..(28): Xaa at residue 7 is Glu or gamma-carboxy Glu; Xaa at residue 5 is Pro or Hyp; Xaa at residue 8 and 19 is Tyr, 125I-Tyr, mono-iodo-Tyr, di-iodo-Tyr, O-sulpho-Tyr or O-phospho-Tyr
TCNTXTXXCTLHQHCCSGXCHKTIQACS

SEQ ID NO: 25 - DNA, 394 bp, Conus arenatus
ggatccatgaaactgacgtgtatggtgatcatcgccgtactgttcctgacggcctgtcaactcattacagctgagacttactccagaggtaagcagatgcaccgcgctctgaggtcaactgacaaaaactcccagttgaccagggaatgcacacctcccggtggagcttgtggtttacctacacactgctgcgggttttgcgatactgcaaacaacagatgtctgtaaagctggtctggcgtctgatattccccttctgtgctctatcctctttggcctgagtcatccgtacctgtgagtggtcatgaactactcaataccctctcctctggaggcttcagaggaactacaatgaaataaaacccgcattgcagagaaaaaaaaaaaaaaaaaa

SEQ ID NO: 26 - protein, 73 aa, Conus arenatus
MKLTCMVIIAVLFLTACQLITAETYSRGKQMHRALRSTDKNSQLTRECTPPGGACGLPTHCCGFCDTANNRCL

SEQ ID NO: 27 - protein, 27 aa, Conus arenatus
  PEPTIDE (1)..(27): Xaa at residue 1 is Glu or gamma-carboxy Glu; Xaa at residue 4, 5 and 12 is Pro or Hyp
XCTXXGGACGLXTHCCGFCDTANNRCL

SEQ ID NO: 28 - DNA, 345 bp, Conus arenatus
  misc_feature (1)..(345): n may be any nucldeotide
ggatccatgaaactgacgtgcgtggtgattatcgccgtgctgttcctgacggcctgtcaactcattacagctgagacttactccagaggtgagcagaatcaccatgttctgaggtcaactgacaaaaactccaagttgaccaggacatgcaacactcccactgaatattgtactttgcatcaacactgctgcagcggccactgccataaaacaatccaggcatgtgcataataccggtgggtggtcatgaaccactcaataccctctcctctggaggcttcagaggaactgcattgaaataaaaccgcattgcaatgaanaaaaaaaaaaaaaaaaaaaaaaaaa

SEQ ID NO: 29 - protein, 74 aa, Conus arenatus
MKLTCVVIIAVLFLTACQLITAETYSRGEQNHHVLRSTDKNSKLTRTCNTPTEYCTLHQHCCSGHCHKTIQACA

SEQ ID NO: 30 - protein, 28 aa, Conus arenatus
  PEPTIDE (1)..(28): Xaa at residue 7 is Glu or gamma-carboxy Glu; Xaa at residue 5 is Pro or Hyp; Xaa at residue8  is Tyr, 125I-Tyr, mono-iodo-Tyr, di-iodo-Tyr, O-sulpho-Tyr or O-phospho-Tyr
TCNTXTXXCTLHQHCCSGHCHKTIQACA

SEQ ID NO: 31 - DNA, 322 bp, Conus arenatus
ggatccatgaaactgacgtgtgtggtgatcatcgccgtgctgttcctgacggcctgtcaactcactacaggtgagcagaaggaccatgctctgaggtcaactgacaaaaactccaagttgactaggcagtgctcgcctatcggtggatattgtactcttcatattcactgctgcagcaaccattgcattaaacctatcggccgatgtgtggcaacctgatacccgtgcgtggtcatgaacccctcaataccctctcctctggaggcttcagaggaactgcattgaaataaaaccgcattgcaataaaaaaaaaaaaaaaaaa

SEQ ID NO: 32 - protein, 70 aa, Conus arenatus
MKLTCVVIIAVLFLTACQLTTGEQKDHALRSTDKNSKLTRQCSPIGGYCTLHIHCCSNHCIKPIGRCVAT

SEQ ID NO: 33 - protein, 30 aa, Conus arenatus
  PEPTIDE (1)..(30): Xaa at residue 1 is Gln or pyro-Glu; Xaa at residue 4 and 23 is Pro or Hyp; Xaa at residue 8 is Tyr, 125I-Tyr, mono-iodo-Tyr, di-iodo-Tyr, O-sulpho-Tyr or O-phospho-Tyr
XCSXIGGXCTLHIHCCSNHCIKXIGRCVAT

SEQ ID NO: 34 - DNA, 318 bp, Conus arenatus
ggatccatgaaactgacgtgcgtggtgatcatcgccgtgctgttcctgacggcctgtcaactcactacaggtgagcagaaggaccatgctctgaggtcaactgacaaaaactccaagttgactaggcagtgcttgcctaacggtggatattgtactcttcatattcactgctgcagcgaccattgcattaaacctatcgaccgatgtgtggcaacctgatacccgggcgtggtcatgaacccctcaataccctctcctctggaggcttcagaggaactgcattgaaataaaaccgcattacaaaaaaaaaaaaaaaaa

SEQ ID NO: 35 - protein, 70 aa, Conus arenatus
MKLTCVVIIAVLFLTACQLTTGEQKDHALRSTDKNSKLTRQCLPNGGYCTLHIHCCSDHCIKPIDRCVAT

SEQ ID NO: 36 - protein, 30 aa, Conus arenatus
  PEPTIDE (1)..(30): Xaa at residue 1 is Gln or pyro-Glu; Xaa at residue 4 and 23 is Pro or Hyp; Xaa at residue 8 is Tyr, 125I-Tyr, mono-iodo-Tyr, di-iodo-Tyr, O-sulpho-Tyr or O-phospho-Tyr
XCLXNGGXCTLHIHCCSDHCIKXIDRCVAT

SEQ ID NO: 37 - DNA, 374 bp, Conus aurisiacus
atgaaactgacgtgtgtggtgatcgtcgccgtgctgctcctgacggcctgtcaactcatcacagctgatgactccagaggtacgcagaagcatcgttccctgagctcggccaccaaactctccatgtcgactcgctgcaagggtaaaggaaaaccatgcagtaggatttcgtataactgctgcaccggttcttgcagatcaggtaaatgtggctgatccagcgcctgatcttcccccttctgtgctctatccttttctgcctgagtcctccttacctgagagtggtcatgaaccactcatcacctgctcctctggaggccccagaggagctacattgaaataaaagtcgcattgcaaaaaaaaaaaaaaaaaaa

SEQ ID NO: 38 - protein, 71 aa, Conus aurisiacus
MKLTCVVIVAVLLLTACQLITADDSRGTQKHRSLSSATKLSMSTRCKGKGKPCSRISYNCCTGSCRSGKCG

SEQ ID NO: 39 - protein, 25 aa, Conus aurisiacus
  PEPTIDE (1)..(25): Xaa at residue 7 is Pro or Hyp; Xaa at residue 13 is Tyr, 125I-Tyr, mono-iodo-Tyr, di-iodo-Tyr, O-sulpho-Tyr or O-phospho-Tyr
CKGKGKXCSRISXNCCTGSCRSGKC

SEQ ID NO: 40 - DNA, 380 bp, Conus aurisiacus
atgaaactgacgtgtgtggtgatcgtcgccgtgctgctcctgacggcctgtcaactcatcacagctgatgactccagaggtacgcagaagcatcgttccctgaggtcgaagaccaaactctccatgtcgactggctgcatggaagccggatcttattgcggctctactacgagaatctgctgcggtttttgcgcttatttcggcaaaaaatgtattgactatcccagcaactgatcttccccctactgtgctctatccttttctgcctgagtcctccttacctgagagtggtcatgaaccactcatcacctgctcctctggaggccccagaggagctacattgaaataaaatcgcattgctaaaaaaaaaaaaaaaaaaa

SEQ ID NO: 41 - protein, 77 aa, Conus aurisiacus
MKLTCVVIVAVLLLTACQLITADDSRGTQKHRSLRSKTKLSMSTGCMEAGSYCGSTTRICCGFCAYFGKKCIDYPSN

SEQ ID NO: 42 - protein, 32 aa, Conus aurisiacus
  PEPTIDE (1)..(32): Xaa at residue 3 is Glu or gamma-carboxy Glu; Xaa at residue 30 is Pro or Hyp; Xaa at residue 7, 21 and 29 is Tyr, 125I-Tyr, mono-iodo-Tyr, di-iodo-Tyr, O-sulpho-Tyr or O-phospho-Tyr
CMXAGSXCGSTTRICCGFCAXFGKKCIDXXSN

SEQ ID NO: 43 - DNA, 373 bp, Conus aurisiacus
accaaaaccatcatcaaaatgaaactgacgtgtgtggtgatcgtcgccgtgctgctcctgacggcctgtcaactcatcacagctgatgactccagaggtacgcagaagcatcgttccctgagctcggccaccaaactctccatgtcgactcgctgcaaggctaaaggaaaaccatgcagtaggattgcgtataactgctgcaccggttcttgcagatcaggtaaatgtggctgatccagtgcctgatcttcccccttctgtgctctatccttttctgcctgagtcctccttacctgagagtggtcatgaaccactcatcacctgctcctctggaggccccagaggagctacattgaaataaaagccgcattgc

SEQ ID NO: 44 - protein, 71 aa, Conus aurisiacus
MKLTCVVIVAVLLLTACQLITADDSRGTQKHRSLSSATKLSMSTRCKAKGKPCSRIAYNCCTGSCRSGKCG

SEQ ID NO: 45 - protein, 25 aa, Conus aurisiacus
  PEPTIDE (1)..(25): Xaa at residue 7 is Pro or Hyp; Xaa at residue 13 is Tyr, 125I-Tyr, mono-iodo-Tyr, di-iodo-Tyr, O-sulpho-Tyr or O-phospho-Ty
CKAKGKXCSRIAXNCCTGSCRSGKC

SEQ ID NO: 46 - DNA, 379 bp, Conus aurisiacus
accaaaaccatcatcaaaatgaaactgacgtgtgtggtgatcgtcgccgtgctgctcctgacgacctgtcaactcatcacagctgatgactccagaggtacgcaggagcatcgtgccctgaggtcgaagacaaaactctccatgttaactttgcgctgcgcatcttacggaaaaccttgtggtattgacaacgactgctgcaatgcatgcgatccaggaagaaatatatgtacgtagctgatccagcgcctgatcttcccccttctgtgctctatccttttctgcccgagtcctccttacctgagagtggtcatgaaccactcatcacctgctccctggaggcctcagaggagctacaatgaaataaaagccgcattgc

SEQ ID NO: 47 - protein, 72 aa, Conus aurisiacus
MKLTCVVIVAVLLLTTCQLITADDSRGTQEHRALRSKTKLSMLTLRCASYGKPCGIDNDCCNACDPGRNICT

SEQ ID NO: 48 - protein, 26 aa, Conus aurisiacus
  PEPTIDE (1)..(26): Xaa at residue 7 and 20 is Pro or Hyp; Xaa at residue 4 is Tyr, 125I-Tyr, mono-iodo-Tyr, di-iodo-Tyr, O-sulpho-Tyr or O-phospho-Tyr
CASXGKXCGIDNDCCNACDXGRNICT

SEQ ID NO: 49 - DNA, 382 bp, Conus bullatus
accaaaaccatcatcaaaatgaaactgacgtgtgtggcgatcgtcgccgtgctgctcctgacggcctgtcagctcattacagctgaagactccagaggtacgcatgagcatcttgccctgaagtcgacctccaaagtctccaagtcgactagctgcatggaagccggatcttattgcggacctgctactacgaaaatctgctgcgatttttgcagtccattcagcgatagatgtatgaacaatcccaacaattgatcttcccccttgtgtgctccatccttttctgcctgagtcctccttacctgagagtggtcatgaaccactcatcacctactcctctggaggcttcagaggagctacattgaaataaaagccgcattgc

SEQ ID NO: 50 - protein, 78 aa, Conus bullatus
MKLTCVAIVAVLLLTACQLITAEDSRGTHEHLALKSTSKVSKSTSCMEAGSYCGPATTKICCDFCSPFSDRCMNNPNN

SEQ ID NO: 51 - protein, 36 aa, Conus bullatus
  PEPTIDE (1)..(36): Xaa at residue 6 is Glu or gamma-carboxy Glu; Xaa at residue 13,25 and 34 is Pro or Hyp; Xaa at residue10  is Tyr, 125I-Tyr, mono-iodo-Tyr, di-iodo-Tyr, O-sulpho-Tyr or O-phospho-Tyr
STSCMXAGSXCGXATTKICCDFCSXFSDRCMNNXNN

SEQ ID NO: 52 - DNA, 400 bp, Conus bullatus
accaaaaccatcatcaaaatgaaactgacgtgtgtggtgatcgtcgccgtgctgctcctgacggcctgtcagctcattacagctgaagactccagaggtacgcagttgcatcgtgccctgaggaaggccaccaaacaccctgtgtcgactcgctgcattactccaggaacacgatgtaaggttccgagccaatgctgcagaggtccttgcaagaacggtcgttgtactccatccccttctgaatggtaaatgtggttgatccagcgcctgatcttcccccttcgtcgtgctccatccttttctgcctgagtcctccttacctgagagtggtcatgaaccactcatcacctactcccctggaggcttcagaggagctacattgaaataaaagccgcattgc

SEQ ID NO: 53 - protein, 76 aa, Conus bullatus
MKLTCVVIVAVLLLTACQLITAEDSRGTQLHRALRKATKHPVSTRCITPGTRCKVPSQCCRGPCKNGRCTPSPSEW

SEQ ID NO: 54 - protein, 31 aa, Conus bullatus
  PEPTIDE (1)..(31): Xaa at residue 30 is Glu or gamma-carboxy Glu; Xaa at residue 4, 11, 18, 26 and 28 is Pro or Hyp; Xaa at residue 31is Trp or Bromo Trp
CITXGTACKVXSQCCRGXCKNGRCTXSXSXX

SEQ ID NO: 55 - DNA, 379 bp, Conus bullatus
accaaaaccatcatcaaaatgaaactgacgtgtgtggcgatcgtcgccgtgctgctcctgacggcctgtcagctcattacagctgaggactccagagatacgcagaagcatcgtgccctgaggtcggacaccaaactctccatgttgactttgcgctgcgcaacttacggaaaaccttgtggtattcaaaacgactgctgcaatacatgcgatccagccagaaggacatgtacgtagctgatccggcgtcttgatcctccgcttctgtgctccatcttttctgcctgagtcctccttacctgagagtggtcatgaaccactcatcacctactcctctggaggctttagaggagctacattgaaataaaagccgcattgc

SEQ ID NO: 56 - protein, 72 aa, Conus bullatus
MKLTCVAIVAVLLLTACQLITAEDSRDTQKHRALRSDTKLSMLTLRCATYGKPCGIQNDCCNTCDPARRTCT

SEQ ID NO: 57 - protein, 26 aa, Conus bullatus
  PEPTIDE (1)..(26): Xaa at residue 7 and 20 is Pro or Hyp; Xaa at residue 4 is Tyr, 125I-Tyr, mono-iodo-Tyr, di-iodo-Tyr, O-sulpho-Tyr or O-phospho-Tyr
CATXGKXCGIQNDCCNTCDXARRTCT

SEQ ID NO: 58 - DNA, 373 bp, Conus bullatus
accaaaaccatcatcaaaatgaaactgacgtgtgtggcgatcgtcgccgtgctgctcctgacggcctgtcagctcattacagctgaagactccagaggtacgcagttgcatcgtgccctgaggaagaccaccaaactctccttgtcgactcgctgcaagggtccaggagcatcatgtataaggattgcgtataactgctgcaagtattcttgcagaaatggtaaatgtggctgatccagcgcctgatcttcccccttgtgtgctccatccttttctgcctgagtcctccttacctgagagtggtcatgaaccactcatcacctactcctctggaggcttcagaggagctacattgaaataaaagccgcattgc

SEQ ID NO: 59 - protein, 71 aa, Conus bullatus
MKLTCVAIVAVLLLTACQLITAEDSRGTQLHRALRKTTKLSLSTRCKGPGASCIRIAYNCCKYSCRNGKCG

SEQ ID NO: 60 - protein, 25 aa, Conus bullatus
  PEPTIDE (1)..(25): Xaa at residue 4 is Pro or Hyp; Xaa at residue 13 and 18 is Tyr, 125I-Tyr, mono-iodo-Tyr, di-iodo-Tyr, O-sulpho-Tyr or O-phospho-Tyr
CKGXGASCIRIAXNCCKXSCRNGKC

SEQ ID NO: 61 - DNA, 382 bp, Conus bullatus
atcaaaaccatcatcaaaatgaaactgacgtgtgtggtgatcgtcgccgtgctgctcctgacggcctgtcagctcattacagctgaagactccagaggtacgcatgagcatcttgccctgaagtcgacctccaaagtctccaagtcgactagctgcatggcagccggatcttattgcggacctgctactacgaatatctgctgcgatttttgcagtccattcagcgatagatgtatgaaaaagcccaacaattgatcttcccccttctgtgctctatccttttctgcctgagtcctccttacctgagagtggtcatgaaccactcatcacctactcctctggaggcttcagaggagctacattgaaataaaagccgcattgc

SEQ ID NO: 62 - protein, 78 aa, Conus bullatus
MKLTCVVIVAVLLLTACQLITAEDSRGTHEHLALKSTSKVSKSTSCMAAGSYCGPATTNICCDFCSPFSDRCMKKPNN

SEQ ID NO: 63 - protein, 36 aa, Conus bullatus
  PEPTIDE (1)..(36): Xaa at residue 13, 25 and 34 is Pro or Hyp; Xaa at residue 10 is Tyr, 125I-Tyr, mono-iodo-Tyr, di-iodo-Tyr, O-sulpho-Tyr or O-phospho-Tyr
STSCMAAGSXCGXATTNICCDFCSXFSDRCMKKXNN

SEQ ID NO: 64 - DNA, 373 bp, Conus bullatus
accaaaaccatcatcaaaatgaaactgacgtgtgtggtgatcgtcgccgtgctgctcctgacggcctgtcagctcattatagctgaggactccagaggtacgcagttgcatcgtgccctgaggaaggccaccaaactctccgtgtcgactcgctgcaagagtaaaggatcatcatgtcataggacttcgtatgactgctgcacgggttcttgcagaaatggtagatgtggctgatccagcgcctgatcttcccccttctgtgctccatccttttctgcctgagtcctccttacctgagagtggtcatgaaccactcatcacctactcctctggaggcttcagaggagctacattgaaataaaagccgcattgc

SEQ ID NO: 65 - protein, 71 aa, Conus bullatus
MKLTCVVIVAVLLLTACQLIIAEDSRGTQLHRALRKATKLSVSTRCKSKGSSCHRTSYDCCTGSCRNGRCG

SEQ ID NO: 66 - protein, 25 aa, Conus bullatus
  PEPTIDE (1)..(25): Xaa at residue 13 is Tyr, 125I-Tyr, mono-iodo-Tyr, di-iodo-Tyr, O-sulpho-Tyr or O-phospho-Tyr
CKSKGSSCHRTSXDCCTGSCRNGRC

SEQ ID NO: 67 - DNA, 321 bp, Conus caracteristicus
ggatccatgaaactgacgtgcgtggtgatcatcgccgtgctgttcctgacggcctgtcaactcattacaggtgagcagaaggaccatgctctgaggtcaactgacaaaaactccaagttgactaggcagtgctcggctaacggtggatcttgtactcgtcattttcactgctgcagcctctattgcaataaagattccagtgtatgtgtggcaacctcatacccgtgagtggccatgaacccctcaataccctctcctctggaggcttcagaggaactgcattgaaataaaaccgcattacaaaaaaaaaaaaaaaaaaaa

SEQ ID NO: 68 - protein, 73 aa, Conus caracteristicus
MKLTCVVIIAVLFLTACQLITGEQKDHALRSTDKNSKLTRQCSANGGSCTRHFHCCSLYCNKDSSVCVATSYP

SEQ ID NO: 69 - protein, 33 aa, Conus caracteristicus; identical to SEQ ID NO: 15
  PEPTIDE (1)..(33): Xaa at residue 1 is Gln or pyro-Glu; Xaa at residue 33 is Pro or Hyp; Xaa at residue 19 and 32 is Tyr, 125I-Tyr, mono-iodo-Tyr, di-iodo-Tyr, O-sulpho-Tyr or O-phospho- Tyr

SEQ ID NO: 70 - protein, 26 aa, Conus catus
CKSTGASCRRTSYDCCTGSCRSGRCG

SEQ ID NO: 71 - protein, 25 aa, Conus catus
  PEPTIDE (1)..(25): Xaa at residue 13 is Tyr, 125I-Tyr, mono-iodo- Tyr, di-iodo-Tyr, O-sulpho-Tyr or O-phospho-Tyr
CKSTGASCRRTSXDCCTGSCRSGRC

SEQ ID NO: 72 - DNA, 229 bp, Conus catus
tcgactcgctgccagggtagaggagcatcatgtcgtaagactatgtataactgctgcagcggttcttgcaacagaggtagttgtggctgatccggcgcctgatcttcccccttccgtgctctatccttttctgcctgattcctccttacctgagagcggtcatgaaccactcatcacctgctcctctggaggcctcagaggagctacattgaaataaaagccgcattgc

SEQ ID NO: 73 - protein, 29 aa, Conus catus
STRCQGRGASCRKTMYNCCSGSCNRGSCG

SEQ ID NO: 74 - protein, 25 aa, Conus catus
  PEPTIDE (1)..(25): Xaa at residue 13 is Tyr, 125I-Tyr, mono-iodo- Tyr, di-iodo-Tyr, O-sulpho-Tyr or O-phospho-Tyr
CQGRGASCRKTMXNCCSGSCNRGSC

SEQ ID NO: 75 - DNA, 235 bp, Conus catus
tcgacacgctgcttgcctgccggagagtcttgcctttttagtaggattagatgctgcggtacttgcagttcagtcttaaagtcatgtgtgagctgatccagctgctgatcttcctcctcctgtgctccatccttttctgcctgagtcctccttatctgagagtggtcatgaaccactcaccacctactcttctggaggcttcagaggagctacagtgaaataaaagccgcattgc

SEQ ID NO: 76 - protein, 31 aa, Conus catus
STRCLPAGESCLFSRIRCCGTCSSVLKSCVS

SEQ ID NO: 77 - protein, 28 aa, Conus catus
  PEPTIDE (1)..(28): Xaa at residue 6 is Glu or gamma-carboxy Glu; Xaa at residue 3 is Pro or Hyp
CLXAGXSCLFSRIRCCGTCSSVLKSCVS

SEQ ID NO: 78 - DNA, 227 bp, Conus catus
tcgacacgctgccagggtagaggaggaccatgtactaaggctgtgtttaactgctgcagcggttcttgcaacagaggtagatgtggctgatccagcgcctgatcttcccccttctgtgctctatccttttctgcctgagtcctccttactgagagtagtcatgaaccactcatcacctactcctctggaggcctcagagagctacattgaaataaaagccgcattgc

SEQ ID NO: 79 - protein, 29 aa, Conus catus
STRCQGRGGPCTKAVFNCCSGSCNRGRCG

SEQ ID NO: 80 - protein, 25 aa, Conus catus
  PEPTIDE (1)..(25): Xaa at residue 7 is Pro or Hyp
CQGRGGXCTKAVFNCCSGSCNRGRC

SEQ ID NO: 81 - DNA, 236 bp, Conus catus
ttaactttgcgctgcgcaacttacggaaaaccttgtggtattcaaaacgactgctgcaatacatgcgatccagccagaaagacatgtacgtagctgatccggcgtctgatctccccccttctgtgctctatccttttctgcctgagtcctccttacctgagagtggtcatgaaccactcatcacctgctcctctggaggcctcgggggagctacattgaaataaaagccgcattgc

SEQ ID NO: 82 - protein, 30 aa, Conus catus
LTLRCATYGKPCGIQNDCCNTCDPARKTCT

SEQ ID NO: 83 - protein, 26 aa, Conus catus
  PEPTIDE (1)..(26): Xaa at residue 7 and 20 is Pro or Hyp; Xaa at residue 4 is Tyr, 125I-Tyr, mono-iodo-Tyr, di-iodo- Tyr, O-sulpho-Tyr or O-phospho-Tyr
CATXGKXCGIQNDCCNTCDXARKTCT

SEQ ID NO: 84 - DNA, 229 bp, Conus catus
tcgactcgctgccggggtagaggaggaccatgtactaaggctatgtttaactgctgcagcggttcttgcaacagaggtagatgtggctgatccagcgcctgatcttcccccttctgtgctctatccttttctgcctgagtcctccttaactgagagtagtcatgaaccactcatcacctactcctctggaggcctcagagaagcatcattgaaataaaagccgcattgc

SEQ ID NO: 85 - protein, 29 aa, Conus catus
STRCRGRGGPCTKAMFNCCSGSCNRGRCG

SEQ ID NO: 86 - protein, 25 aa, Conus catus
  PEPTIDE (1)..(25): Xaa at residue 7 is Pro or Hyp
CRGRGGXCTKAMFNCCSGSCNRGRC

SEQ ID NO: 87 - DNA, 374 bp, Conus circumcisus
accaaaaccatcatcaaaatgaaactgacgtgtgtggtgatcgtcgccgtgctgctcctgacgacctgtcaactcatcacagctgatgactccagaggtacgcaggagcatcgtgccctgaggtcggacaccaaactccccatgtcgactcgctgcaagggtaaaggagcatcatgtcgtaagactatgtataactgctgcagcggttcttgcagcaacggtagatgtggctgatccagcgcctgatcttcccccttctgctgctctatccttttctgcctgagtcctccttacctgagagctggtcatgaaccactcatcacctgctcctctggaggcccagaggagctacattgaaataaaagccgcattgc

SEQ ID NO: 88 - protein, 71 aa, Conus circumcisus
MKLTCVVIVAVLLLTTCQLITADDSRGTQEHRALRSDTKLPMSTRCKGKGASCRKTMYNCCSGSCSNGRCG

SEQ ID NO: 89 - protein, 25 aa, Conus circumcisus
  PEPTIDE (1)..(25): Xaa at residue 13 is Tyr, 125I-Tyr, mono-iodo- Tyr, di-iodo-Tyr, O-sulpho-Tyr or O-phospho-Tyr
CKGKGASCRKTMXNCCSGSCSNGRC

SEQ ID NO: 90 - DNA, 379 bp, Conus circumcisus
accaaaaccatcatcaaaatgaaactgacgtgtgtggtgatcgtcgccgtgctgctcctgacgacctgtcaactcatcacagctgatgactccagaggtacgcagaagcatcgtgccctgaggtcggccaccaaagtctccaagtcgactagctgcatggaagccggatcttattgccgctctactacgagaacctgctgcggttattgctcttatttcagcaaaaaatgtattgactttcccagcaactgatcttccccctactgtgctctatccttttctgcctgagtcctccttacctgagagtggtcatgaaccactcatcaccctactcctctggaggcccagaggagctacattgaaataaaagccgcattgc

SEQ ID NO: 91 - protein, 77 aa, Conus circumcisus
MKLTCVVIVAVLLLTTCQLITADDSRGTQKHRALRSATKVSKSTSCMEAGSYCRSTTRTCCGYCSYFSKKCIDFPSN

SEQ ID NO: 92 - protein, 35 aa, Conus circumcisus
  PEPTIDE (1)..(35): Xaa at residue 6 is Glu or gamma-carboxy Glu; Xaa at residue 33 is Pro or Hyp; Xaa at residue 10, 21 and 24 is Tyr, 125I-Tyr, mono-iodo-Tyr, di-iodo-Tyr, O-sulpho-Tyr or O-phospho-Tyr
STSCMXAGSXCRSTTRTCCGXCSXFSKKCIDFXSN

SEQ ID NO: 93 - DNA, 379 bp, Conus circumcisus
accaaaaccatcatcaaaatgaaactgacgtgtgtggtgatcgtcgccgtgctgctcctgacgacctgtcaactcatcacagctgatgactccagaggtacgcaggagcatcgtgccctgaggtcggacaccaaactccccatgtcgactcgctgcaagagtaaaggagcaaaatgttcaaggcttatgtatgactgctgcagcggttcttgcagcaggtactcaggtagatgtggctgatccagcgcctgatcttcccccttctgctgctctatccttttctgcctgagtcctccttacctgagagtggtcatgaaccactcatcacctactcctctggaggcccagaggagctacattgaaataaaagccgcattgc

SEQ ID NO: 94 - protein, 73 aa, Conus circumcisus
MKLTCVVIVAVLLLTTCQLITADDSRGTQEHRALRSDTKLPMSTRCKSKGAKCSRLMYDCCSGSCSRYSGRCG

SEQ ID NO: 95 - protein, 27 aa, Conus circumcisus
  PEPTIDE (1)..(27): Xaa at residue 13 and 23 is Tyr, 125I-Tyr, mono-iodo-Tyr, di-iodo-Tyr, O-sulpho-Tyr or O-phospho-Tyr
CKSKGAKCSRLMXDCCSGSCSRXSGRC

SEQ ID NO: 96 - DNA, 379 bp, Conus circumcisus
accaaaaccatcatcaaaatgaaactgacgtgtgtggtgatcgtcgccgtgctgctcctgacgacctgtcaactcatcacagctgatgactccagaggtacgcagaagcatcgttccctgacgtcggccaccaaagtctccaagtcgactggctgcatgaaagccggatcttattgccgctctactacgagaacttgctgcggttattgcgcttatttcggcaaaaaatgtattgactatcccagcaactgatcttccccctactgtgctctatccttttctgcctaagtcctccttacctgagagtggtcatgaaccactcatcaccctactcctctggaggcccagaggagctacattgaaataaaagccgcattgc

SEQ ID NO: 97 - protein, 77 aa, Conus circumcisus
MKLTCVVIVAVLLLTTCQLITADDSRGTQKHRSLTSATKVSKSTGCMKAGSYCRSTTRTCCGYCAYFGKKCIDYPSN

SEQ ID NO: 98 - protein, 35 aa, Conus circumcisus
  PEPTIDE (1)..(35): Xaa at residue 33 is Pro or Hyp; Xaa at residue 10, 21, 24 and 32 is Tyr, 125I-Tyr, mono-iodo-Tyr, di-iodo-Tyr, O-sulpho-Tyr or O-phospho-Tyr
STGCMKAGSXCRSTTRTCCGXCAXFGKKCIDXXSN

SEQ ID NO: 99 - DNA, 362 bp, Conus consors
atgaaactgacgtgtgtggtgatcgtcgccgtgctgctcctgacggcctgtcaactcctcacagctgatgactccagaggtacgcagaagcatcgtgccctgaagtcttacaccaaactctccatgttaactttgcgctgcgcatcttacggaaaaccttgtggtattgacaacgactgctgcaatacatgcgatccagccagaaagacatgtacgtagctgatccggcgtctgatcttcccccttctgtgctctatccttttctgcctgagtcctccttacctgagagtggtcatgaaccactcatcacctagctcctctggaggcttcagaggagctacaatgaaataaaagcgcattgc

SEQ ID NO: 100 - protein, 72 aa, Conus consors
MKLTCVVIVAVLLLTACQLLTADDSRGTQKHRALKSYTKLSMLTLRCASYGKPCGIDNDCCNTCDPARKTCT

SEQ ID NO: 101 - protein, 26 aa, Conus consors
  PEPTIDE (1)..(26): Xaa at residue 7 and 20 is Pro or Hyp; Xaa at residue 4 is Tyr, 125I-Tyr, mono-iodo-Tyr, di-iodo- Tyr, O-sulpho-Tyr or O-phospho-Tyr
CASXGKXCGIDNDCCNTCDXARKTCT

SEQ ID NO: 102 - DNA, 237 bp, Conus consors
atgaaactgacgtgtgtggtgatcgtcgccgtgctgctcctgacggcctgtcaactcctcacagctgatgactccagaggtacgcagaagcatcgtgccctgaggtcggacaccaaactctccatgtcgactcgctgcaagggtacaggaaaaccatgcagtaggattgcgtataactgctgcaccggttcttgcagatcaggtaaatgtggctgatccagcgcctgatctcccccc

SEQ ID NO: 103 - protein, 71 aa, Conus consors
MKLTCVVIVAVLLLTACQLLTADDSRGTQKHRALRSDTKLSMSTRCKGTGKPCSRIAYNCCTGSCRSGKCG

SEQ ID NO: 104 - protein, 25 aa, Conus consors
  PEPTIDE (1)..(25): Xaa at residue 7 is Pro or Hyp; Xaa at residue 13 is Tyr, 125I-Tyr, mono-iodo-Tyr, di-iodo-Tyr, O-sulpho-Tyr or O-phospho-Tyr
CKGRGKXCSRIAXNCCTGSCRSGKC

SEQ ID NO: 105 - DNA, 320 bp, Conus consors
atgaaactgacgtgtgtggtgatcgtcgccgtgctgctcctgacggcctgtcaactcatcacagctgatgactccaaaggtacgcagaagcatcgttccctgaggtcgaccaccaaagtctccaaggcgactgactgcattgaagccggaaattattgcggacctactgttatgaaaatctgctgcggcttttgcagtccatacagcaaaatatgtatgaactatccccaaaattgatcttcccccttctgtgctctatccttttctgcctgagtcctccttacctgagagtggtcatgaaccactcatcacctcgtccc

SEQ ID NO: 106 - protein, 78 aa, Conus consors
MKLTCVVIVAVLLLTACQLITADDSKGTQKHRSLRSTTKVSKATDCIEAGNYCGPTVMKICCGFCSPYSKICMNYPQN

SEQ ID NO: 107 - protein, 36 aa, Conus consors
  PEPTIDE (1)..(36): Xaa at residue 6 is Glu or gamma-carboxy Glu; Xaa at residue 13, 25 and 34 is Pro or Hyp; Xaa at residue 10, 26 and 33 is Tyr, 125I-Tyr, mono-iodo-Tyr, di-iodo-Tyr, O-sulpho- Tyr or O-phospho-Tyr
ATDCIXAGNXCGXTVMKICCGFCSXXSKICMNXXQN

SEQ ID NO: 108 - DNA, 321 bp, Conus consors
atgaaactgacgtgtgtggtgatcgtcgccgtgctgctcctgacggcctgtcaactcctcacagctgatgactccagaggtacgcagaagcatcgtgccctgaggtcggacaccaaactctccatgtcgactcgctgcaaaggtaaaggagcatcatgtacaaggcttatgtatgactgctgccacggttcttgcagcagcagcaagggtagatgtggctgatccggcgcctgatcttcccccttctgtgctctatccttttctgcctgagtcctccttacctgagaggtggtcatgaaccactcatcacctgctcccctg

SEQ ID NO: 109 - protein, 73 aa, Conus consors
MKLTCVVIVAVLLLTACQLLTADDSRGTQKHRALRSDTKLSMSTRCKGKGASCTRLMYDCCHGSCSSSKGRCG

SEQ ID NO: 110 - protein, 27 aa, Conus consors
  PEPTIDE (1)..(27): Xaa at residue 13 is Tyr, 125I-Tyr, mono- iodo-Tyr, di-iodo-Tyr, O-sulpho-Tyr or O-phospho-Tyr
CKGKGASCTRLMXDCCHGSCSSSKGRC

SEQ ID NO: 111 - DNA, 292 bp, Conus consors
ggatccatgaaactgacgtgcatggtgatcgtcgccgtgctgctcctgacggcctgtcaactcatcacagctgatgactccagaggtacgcagaagcatcgtgccctgaggtcggacaccaaactctccatgtcaactcgctgcaagggtaaaggagcatcatgtcataggacttcgtatgactgctgcaccggttcttgcaacagaggtaaatgtggctgatccggcgcctgatcttcccccttctgtgctctatccttttctgcctgagtcatccatacctgtgctcgag

SEQ ID NO: 112 - protein, 71 aa, Conus consors
MKLTCMVIVAVLLLTACQLITADDSRGTQKHRALRSDTKLSMSTRCKGKGASCHRTSYDCCTGSCNRGKCG

SEQ ID NO: 113 - protein, 25 aa, Conus consors
  PEPTIDE (1)..(25): Xaa at residue 13 is Tyr, 125I-Tyr, mono- iodo-Tyr, di-iodo-Tyr, O-sulpho-Tyr or O-phospho-Tyr
CKGKGASCHRTSXDCCTGSCNRGKC

SEQ ID NO: 114 - DNA, 299 bp, Conus consors
ggatccatgaaactgacgtgcgtggtgatcgtcgccgtgctgctcctgacggcctgtcaactcatcacagctgatgactccagaggtacgcagaagcatcgtgccctgaagtcggacaccaaactctccatgttaactttgcgctgcgcatcttacggaaaaccttgtggtatttacaacgactgctgcaatacatgcgatccagccagaaagacatgtacgtagctgatccggcgtctgatcttcccccttctgtgctctatccttttctgcctgagtcatccatacctgtgctcgag

SEQ ID NO: 115 - protein, 72 aa, Conus consors
MKLTCVVIVAVLLLTACQLITADDSRGTQKHRALKSDTKLSMLTLRCASYGKPCGIYNDCCNTCDPARKTCT

SEQ ID NO: 116 - protein, 26 aa, Conus consors
  PEPTIDE (1)..(26): Xaa at residue 7 and 20 is Pro or Hyp; Xaa at residue 4 and 11 is Tyr, 125I-Tyr, mono-iodo-Tyr, di-iodo- Tyr, O-sulpho-Tyr or O-phospho-Tyr
CASXGKXCGIXNDCCNTCDXARKTCT

SEQ ID NO: 117 - DNA, 434 bp, Conus consors
  misc_feature (1)..(434): n may be any nucleotide
ggatccatgaaactgacgtgtgtggtgatcgtcgccgtgctgctcctgacggcctgtcaactcatcacagctgatgactccagaggtacgcagaagcatcgtgccctgaggtcggacaccaaactctccatgtcgactcgctgcaagggtacaggaaaaccatgcagtagggttgcgtataactgctgcaccggttcttgcagatcaggtaaatgtggctgatccagtgcctgatcttcccccttctgtgctctatccttttctgcctgagtcctccttacctgagagtggtcatgaaccactcatcacctgctcctctggaggcttcagaggagctacattgaaataaaagccgcattgcantgnanaaaannnnnnnnnnnnnnnnnnnnnnnnnnnnnnnnnnnnnnnnggaaaaaaaaaaaaaaaaaaaa

SEQ ID NO: 118 - protein, 71 aa, Conus consors
MKLTCVVIVAVLLLTACQLITADDSRGTQKHRALRSDTKLSMSTRCKGTGKPCSRVAYNCCTGSCRSGKCG

SEQ ID NO: 119 - protein, 25 aa, Conus consors
  PEPTIDE (1)..(25): Xaa at residue 7 is Pro or Hyp; Xaa at residue 13 is Tyr, 125I-Tyr, mono-iodo-Tyr, di-iodo-Tyr, O-sulpho-Tyr or O-phospho-Tyr
CKGTGKXCSRVAXNCCTGSCRSGKC

SEQ ID NO: 120 - DNA, 393 bp, Conus consors
ggatccatgaaactgacgtgcatggtgatcgtcgccgtgctgctcctgacggcctgtcaactcatcacagctgatgactccagaggtacgcagaagcatcgttccctgaggtcgaccaccaaagtctccaagtcgactagctgcatgaaagccgggtcttattgccgctctactacgagaacctgctgcggttattgcgcttatttcggcaaattttgtattgactttcccagcaactgatcttccccctactgtgctctatccttttctgcctctgcctgagtcctccttacctgagagtggtcatgaaccactcatcacctgctcccctggaggcctcagaggagctacaatgaaataaaagccgcattgcaaaaaaaaaaaaaaaaaaaa

SEQ ID NO: 121 - protein, 77 aa, Conus consors
MKLTCMVIVAVLLLTACQLITADDSRGTQKHRSLRSTTKVSKSTSCMKAGSYCRSTTRTCCGYCAYFGKFCIDFPSN

SEQ ID NO: 122 - protein, 35 aa, Conus consors
  PEPTIDE (1)..(35): Xaa at residue 33 is Pro or Hyp; Xaa at residue 10, 21 and 24 is Tyr, 125I-Tyr, mono-iodo-Tyr, di- iodo-Tyr, O-sulpho-Tyr or O-phospho-Tyr
STSCMKAGSXCRSTTRTCCGXCAXFGKFCIDFXSN

SEQ ID NO: 123 - DNA, 361 bp, Conus dalli
accaaaaccatcatcaaaatgaaactgacgtgtgtggtgatcgtcgccgtgctgttcctgacggcctgtcaactcatcacagctgatgactccagaagtacgcagaagcatcgtgctctgaggtcgaccatcaaacactccatgttgactaggagctgcacgcctcccggaggaccttgtggttattataatgactgctgcagtcatcaatgcaatataagcagaaataaatgcgagtagctgatccggcatctgatcttccccttctgtgctcgtcctaacctgagagtggtcatgaaccatcatcacctactcctctggaggcttcagaggagctacatggaaataaaagccgcattgc

SEQ ID NO: 124 - protein, 73 aa, Conus dalli
MKLTCVVIVAVLFLTACQLITADDSRSTQKHRALRSTIKHSMLTRSCTPPGGPCGYYNDCCSHQCNISRNKCE

SEQ ID NO: 125 - protein, 28 aa, Conus dalli
  PEPTIDE (1)..(28): Xaa at residue 28 is Glu or gamma-carboxy Glu; Xaa at residue 4, 5 and 8 is Pro or Hyp; Xaa at residue 11 and 12 is Tyr, 125I-Tyr, mono-iodo-Tyr, di-iodo-Tyr, O-sulpho-Tyr or O-phospho-Tyr
SCTXXGGXCGXXNDCCSHQCNISRNKCX

SEQ ID NO: 126 - DNA, 350 bp, Conus distans
accaaaaccatcatcaaaatgaaactgacgtgcgtgttgatcatcgccgtgctgttcctgacggcctgtcaactcactagaggaaagctggagcgtcctgttctgaggtcgagcgaccaaacctccgggtcaacgaagagatgcgaagatcctggtgaaccttgcggaagtgatcattcctgctgcggcggtagttgcaaccacaacgtctgcgcctgaagctggtctggcatctgaccattccccttctgtactctatctctattgcctgagtcatctttacctgtgagtggtcatgaatctctcaataccttctcccctggaggcttcagaagaactagattgaaata

SEQ ID NO: 127 - protein, 66 aa, Conus distans
MKLTCVLIIAVLFLTACQLTRGKLERPVLRSSDQTSGSTKRCEDPGEPCGSDHSCCGGSCNHNVCA

SEQ ID NO: 128 - protein, 25 aa, Conus distans
  PEPTIDE (1)..(25): Xaa at residue 2 and 6 is Glu or gamma-carboxy Glu; Xaa at residue 4 and 7 is Pro or Hyp
CXDXGXXCGSDHSCCGGSCNHNVCA

SEQ ID NO: 129 - DNA, 309 bp, Conus ermineus
atgaaactgacgtgtgtggtgatcgtcgccgtgctgctcctgacggcctgtcaactcatcacagctgacgactccagacgtacgcagaagcatcgtgccctgaggtcgaccaccaaacgcgccacgtcgaatcgcccctgcaagccgaaaggacgaaaatgttttccgcatcagaaggactgctgcaataaaacgtgcaccagatcaaaatgtccctgatcttcccccttctgtgctgtatccttttctgcctgagtcctccttacctgagagtggtcagtaaccactcatcaccatctcctctggagg

SEQ ID NO: 130 - protein, 72 aa, Conus ermineus
MKLTCVVIVAVLLLTACQLITADDSRRTQKHRALRSTTKRATSNRPCKPKGRKCFPHQKDCCNKTCTRSKCP

SEQ ID NO: 131 - protein, 27 aa, Conus ermineus
  PEPTIDE (1)..(27): Xaa at residue 1, 4, 11 and 27 is Pro or Hyp
XXKXKGRKCFXHQKDCCNKTCTRSKCX

SEQ ID NO: 132 - DNA, 308 bp, Conus ermineus
aactcatcacagctgatgactccagaggtacgcagaacgatcgtgccctgaggtcgaccaccaaactctccatgctgactcgggcctgctggtcttccggaacaccttgtggtactgatagtttatgctgcggtggatgcaatgtatccaaaagtaaatgtaactagctgattcggcgtctgaacttcccccttctgtgctctatccttttctgcccgagtcctccatacctgagaatggtcatgaaccactcatcacctactcctctggagacctcagaagagctacactgaaataaaagcgcttgc

SEQ ID NO: 133 - protein, 54 aa, Conus ermineus
LITADDSRGTQNDRALRSTTKLSMLTRACWSSGTPCGTDSLCCGGCNVSKSKCN

SEQ ID NO: 134 - protein, 27 aa, Conus ermineus
  PEPTIDE (1)..(27): Xaa at 8 residue is Pro or Hyp; Xaa at residue 3 is Trp or Bromo Trp
ACXSSGTXCGTDSLCCGGCNVSKSKCN

SEQ ID NO: 135 - DNA, 385 bp, Conus geographus
ggatccatgaaactgacgtgcgtggtgatcgtcgccgtgctgctcctgacggcctgtcaactcatcacagctgatgactccagaggtacgcagaagcatcgtgccctggggtcgaccaccgaactctccttgtcgactcgctgcaagtcacccggatcttcatgttcaccgactagttataattgctgcaggtcttgcaatccatacgccaaaagatgttacggctaatccagcgcctgatcttcccccttctgtgctctatcccttcctgtctgagtcctccttacctgagagtggtcatgaaccactcctcacctacttctctggaggcttcggaggagctacattgaaataaaagccgcattgtaaaaaaaaaaaaaaaaaa

SEQ ID NO: 136 - protein, 73 aa, Conus geographus
MKLTCVVIVAVLLLTACQLITADDSRGTQKHRALGSTTELSLSTRCKSPGSSCSPTSYNCCRSCNPYAKRCYG

SEQ ID NO: 137 - protein, 27 aa, Conus geographus
  PEPTIDE (1)..(27): Xaa at residue 4, 10 and 21 is Pro or Hyp; Xaa at residue 13, 22 and 27 is Tyr, 125I-Tyr, mono-iodo-Tyr, di-iodo- Tyr, O-sulpho-Tyr or O-phospho-Tyr
CKSXGSSCSXTSXNCCRSCNXXAKRCX

SEQ ID NO: 138 - DNA, 396 bp, Conus geographus
ggatccatgaaactgacgtgtgtggtgatcgtcgccgtgctgctcctgacggcctgtcaactcatcacagctgatgactccagaggtacgcagaagcatcgtgccctgaggtcgtccaccaaactcaccttgtcgactcgctgcaaatcacccggaactccatgttcaaggggtatgcgtgattgctgcacgccttgcttgttatacagcaacaaatgtaggcgctactaacccagcgcctgatcttcccccttctgtgctctattcctttctgcctgagtcctccttacctgaaagtggtcatgaaccactcatcacctacttctctggaggcttcagaagagctacattgaaataaaagccgcattgcaatgacaaaaaaaaaaaaaaaaaaaa

SEQ ID NO: 139 - protein, 74 aa, Conus geographus
MKLTCVVIVAVLLLTACQLITADDSRGTQKHRALRSSTKLTLSTRCKSPGTPCSRGMRDCCTPCLLYSNKCRRY

SEQ ID NO: 140 - protein, 29 aa, Conus geographus
  PEPTIDE (1)..(29): Xaa at residue 4, 7 and 18 is Pro or Hyp; Xaa at residue 22 and 29 is Tyr, 125I-Tyr, mono-iodo-Tyr, di-iodo-Tyr, O-sulpho-Tyr or O-phospho-Tyr
CKSXGTXCSRGMRDCCTXCLLXSNKCRRX

SEQ ID NO: 141 - DNA, 407 bp, Conus geographus
ggaattccgtttctgcgctgcttcctttggcatcaccaaaaccatcatcaaaatgaaactgacgtgtgtggtgatcgtcgccgtgctgctcctgacggcctgtcaactcatcacagctgatgactccagaggtacgcagaagcatcgtgccctggggtcgaccaccgaactctccttgtcgactcgctgcaagtcacccggatcttcatgttcaccgactagttataattgctgcaggtcttgcaatccatacaccaaaagatgttacggctaatccagcgcctgatcttccctgctctgagtcctccttacctgagagtggtcatgaaccactcatcacctacttctctaggcggttcggaggagctacattgaaataaaagccgcattgcaaaaaaaaaaaaaaa

SEQ ID NO: 142 - protein, 73 aa, Conus geographus
MKLTCVVIVAVLLLTACQLITADDSRGTQKHRALGSTTELSLSTRCKSPGSSCSPTSYNCCRSCNPYTKRCYG

SEQ ID NO: 143 - protein, 27 aa, Conus geographus
  PEPTIDE (1)..(27): Xaa at residue 4, 10 and 21 is Pro or Hyp; Xaa at residue 13, 22 and 27 is Tyr, 125I-Tyr, mono-iodo-Tyr, di-iodo-Tyr, O-sulpho-Tyr or O-phospho-Tyr
CKSXGSSCSXTSXNCCRSCNXXTKRCX

SEQ ID NO: 144 - protein, 28 aa, Conus geographus
  PEPTIDE (1)..(28): Xaa at residue 4, 10 and 21 is Pro or Hyp; Xaa at residue 13, 22 and 27 is Tyr, 125I-Tyr, mono-iodo-Tyr, di-iodo-Tyr, O-sulpho-Tyr or O-phospho-Tyr
CKSXGSSCSXTSXNCCRSCNXXTKRCXG

SEQ ID NO: 145 - protein, 26 aa, Conus geographus
  PEPTIDE (1)..(26): Xaa at residue 4, 10 and 21 is Pro or Hyp; Xaa at residue 13 and 22 is Tyr, 125I-Tyr, mono-iodo-Tyr, di-iodo-Tyr, O-sulpho-Tyr or O-phospho-Tyr
CKSXGSSCSXTSXNCCRSCNXXTKRC

SEQ ID NO: 146 - DNA, 314 bp, Conus geographus
catcacagctgatgactccagaggtacgcagaagcatcgtgccctgaggtcgtccaccaaactcaccttgtcgactcgctgcaaatcacccggaactccatgttcaaggggtatgcgtgattgctgcacgtcttgcttgttatacagcaacaaatgtaggcgctactaacccagcgcctgatcttcccccttctgtgctctattcctttctgcctgagtcctccttacctgaaagtggtcatgaaccactcatcacctacttctctggaggcttcagaagagctacattgaaataaaagccgcattgcaatgac

SEQ ID NO: 147 - protein, 55 aa, Conus geographus
ITADDSRGTQKHRALRSSTKLTLSTRCKSPGTPCSRGMRDCCTSCLLYSNKCRRY

SEQ ID NO: 148 - protein, 29 aa, Conus geographus
  PEPTIDE (1)..(29): Xaa at residue 4 and 7 is Pro or Hyp; Xaa at residue 22 and 29 is Tyr, 125I-Tyr, mono-iodo-Tyr, di-iodo-Tyr, O-sulpho-Tyr or O-phospho-Tyr
CKSXGTXCSRGMRDCCTSCLLXSNKCRRX

SEQ ID NO: 149 - protein, 29 aa, Conus geographus
  PEPTIDE (1)..(29): Xaa at residue 4 and 7 is Pro or Hyp; Xaa at residue 22 and 29 is Tyr, 125I-Tyr, mono-iodo-Tyr, di-iodo-Tyr, O-sulpho-Tyr or O-phospho-Tyr
CKSXGTXCSRGMRDCCTSCLSXSNKCRRX

SEQ ID NO: 150 - DNA, 380 bp, Conus laterculatus
accaaaaccatcatcaaaatgaaactgacgtgtgtggtgatcgtcgccgtgctgctcctgacggcctgtcaactcatcaccgctgatgactccagaggtacgcagaagcatcgtgccctgaggtcgaccaccaatctctccatgctgactcggaagtgctggccttccggaagctattgtcgtgcgaatagtaaatgctgcagtggatgcgatcggaacagaaataaatgttactagctgattcggcgtctgaacttcctccttctgtgctctatccttttctgcccgagtcctccatacctgagagtggtcatgaaccactcaactcctactcctctggaggcctcagaagagctacattgaaataaaagccgcattgc

SEQ ID NO: 151 - protein, 72 aa, Conus laterculatus
MKLTCVVIVAVLLLTACQLITADDSRGTQKHRALRSTTNLSMLTRKCWPSGSYCRANSKCCSGCDRNRNKCY

SEQ ID NO: 152 - protein, 27 aa, Conus laterculatus
  PEPTIDE (1)..(27): Xaa at residue 4 is Pro or Hyp; Xaa at residue 3 is Trp or Bromo Trp; Xaa at residue 8 and 27 is Tyr, 125I-Tyr, mono-iodo-Tyr, di-iodo-Tyr, O-sulpho-Tyr or O-phospho-Tyr
KCXXSGSXCRANSKCCSGCDRNRNKCX

SEQ ID NO: 153 - DNA, 367 bp, Conus laterculatus
accaaaaccatcatcaaaatgaaactgacgtgtgtggtgatcgtcgccgtgctgctcctgacggcctgtcaactcatcacagctgatgactccagaggtacgcagaagcatcgtgccctgaggtcgaccaccaaactctccatatcgactcgctgccttcctcccggatcatattgtaaggcgacaacggaagtctgctgctcttcttgccttcaattcgctcagatatgttcgggttgatcttccctcttctgtgctctatccttttctgcctgagtcctccatacctgagaatggtcatgaaccactcaacatctactcctctggaggcctcagaagagctatattgaaataaaagccgcattgc

SEQ ID NO: 154 - protein, 73 aa, Conus laterculatus
MKLTCVVIVAVLLLTACQLITADDSRGTQKHRALRSTTKLSISTRCLPPGSYCKATTEVCCSSCLQFAQICSG

SEQ ID NO: 155 - protein, 27 aa, Conus laterculatus
  PEPTIDE (1)..(27): Xaa at residue 13 is Glu or gamma-carboxy Glu; Xaa at residue 3 and 4 is Pro or Hyp; Xaa at residue 7 is Tyr, 125I-Tyr, mono-iodo-Tyr, di-iodo-Tyr, O-sulpho-Tyr or O-phospho-Tyr
CLXXGSXCKATTXVCCSSCLQFAQICS

SEQ ID NO: 156 - DNA, 373 bp, Conus laterculatus
accaaaaccatcatcaaaatgaaactgacgtgtgtggtgatcgtcgccgtgctgctcctgacggcctgtcaactcatcacagctgatgactccagaggtacgcagaagcatcgtgccctgaggtcgaccaccaatctctccatgtcgactcgctgcaagtctcccggatcatcatgtagcgtgtctatgcgtaactgctgcacttcttgcaattcacgcaccaagaaatgtacgcgacgtggctgaacttcccccttctgtgctctatccttttctgcccgagtcctccatacctgagagtggtcatgaaccactcaacatctactcctctggaggcctcagaagagctatattgaaataaaagccgcattgc

SEQ ID NO: 157 - protein, 75 aa, Conus laterculatus
MKLTCVVIVAVLLLTACQLITADDSRGTQKHRALRSTTNLSMSTRCKSPGSSCSVSMRNCCTSCNSRTKKCTRRG

SEQ ID NO: 158 - protein, 29 aa, Conus laterculatus
  PEPTIDE (1)..(29): Xaa at residue 3 is Pro or Hyp
CKSXGSSCSVSMRNCCTSCNSRTKKCTRR

SEQ ID NO: 159 - DNA, 330 bp, Conus laterculatus
accaaaaccatcatcaaaatgaaactgacgtgtgtggtgatcgtcgccgtgctgctcctgacggcctgtcaactcatcacagctgatgactccagaggtacgcagaagcatcgtgccctgaggtcgacaaccaaactctccatgctgactcggacctgctggccttccggaacagcttgtggtattgatagtaactgctgcagtggatgcaatgtatccagaagtaaatgtaactagctgattcggcgtctaaacttcctccttctgcctgagtcctccatacctgagagtggtcatgaaccacatcatcacctcatctctggaggcctc

SEQ ID NO: 160 - protein, 72 aa, Conus laterculatus
MKLTCVVIVAVLLLTACQLITADDSRGTQKHRALRSTTKLSMLTRTCWPSGTACGIDSNCCSGCNVSRSKCN

SEQ ID NO: 161 - protein, 27 aa, Conus laterculatus
  PEPTIDE (1)..(27): Xaa at residue 4 is Pro or Hyp; Xaa at residue 3 is Trp or Bromo Trp
TCXXSGTACGIDSNCCSGCNVSRSKCN

SEQ ID NO: 162 - DNA, 363 bp, Conus laterculatus
accaaaaccatcatcaaaatgaaactgacgtgtgtggtgatcgtcgccgtgctgctcctgacggcctgtcaactcatcacagctgatgactccagaggtacgcagaagcatcgtgccctgaggtcgaccaccaatctctccatgctgactcggaagtgctggccttccggaagctattgtcgtgcgaatagtaaatgctgcagtggatgcgatcggaacagaagtaaatgtaactagctgattcggcgtctaaacttcctccttctgcctgagtcctccatacctgagagtggtcatgaaccactcatcacctactcctctggaggcctcaaaggagctacattgaaataaaagccgcattgc

SEQ ID NO: 163 - protein, 72 aa, Conus laterculatus
MKLTCVVIVAVLLLTACQLITADDSRGTQKHRALRSTTNLSMLTRKCWPSGSYCRANSKCCSGCDRNRSKCN

SEQ ID NO: 164 - protein, 27 aa, Conus laterculatus
  PEPTIDE (1)..(27): Xaa at residue4  is Pro or Hyp; Xaa at residue 3 is Trp or Bromo Trp; Xaa at residue 8 is Tyr, 125I-Tyr, mono- iodo-Tyr, di-iodo-Tyr, O-sulpho-Tyr or O-phospho-Tyr
KCXXSGSXCRANSKCCSGCDRNRSKCN

SEQ ID NO: 165 - DNA, 391 bp, Conus leopardus
  misc_feature (1)..(391): n may be any nucleotide
atgaaactgacgtgtgtggtgatcgtagctgtgctgttcctgacggcctgtcaactcactacagctgacatctccagaggtacgcggaagcgtcgtgctctgaggtcgaccaccaaactctccaggtcgctctttgagtgcgcgccttccggtggacgttgtggttttttaaagtcctgctgcgaaggatattgcgatggggaaagcacttcatgtgtgagtggcccatacagcatctgatcttcccgccttcagtgctctatccttttctgcctgagtcctccatacctctgagcggtcatgaaccactcaacacctactcctctggaggcttcagggaactatattaaaataaagccgcattgcaacgaaanaaaaaaaaaaaaaaaaa

SEQ ID NO: 166 - protein, 79 aa, Conus leopardus
MKLTCVVIVAVLFLTACQLTTADISRGTRKRRALRSTTKLSRSLFECAPSGGRCGFLKSCCEGYCDGESTSCVSGPYSI

SEQ ID NO: 167 - protein, 37 aa, Conus leopardus
  PEPTIDE (1)..(37): Xaa at residue 4, 20 and 26 is Glu or gamma- carboxy Glu; Xaa at residue 7 and 34 is Pro or Hyp; Xaa at residue 22 and 35  is Tyr, 125I-Tyr, mono-iodo-Tyr, di-iodo-Tyr, O-sulpho- Tyr or O-phospho-Tyr
SLFXCAXSGGRCGFLKSCCXGXCDGXSTSCVSGXXSI

SEQ ID NO: 168 - DNA, 365 bp, Conus leopardus
atgaaactgacgtgtgtggtgatcgtcgctgtgctgttcctgacggcctgtcaactcactacagctgacatctccagaggtacgtggaagcatcgtggtgtggggtcgaccaccggactctccccgtggcccttggactgcacggctcccagtcaaccttgtggttattttcctaggtgctgtggacattgcgatgtacgcagggtatgtacgagtggctgatccggcgtctgatctttccgccttctgtgctgtatccttttctgcctgagtcctccatacccgtgagtggtcatgaaccactcaacacctactcctctggaggcttcagaggaactatattaaaataaagccgcattgcaatg

SEQ ID NO: 169 - protein, 73 aa, Conus leopardus
MKLTCVVIVAVLFLTACQLTTADISRGTWKHRGVGSTTGLSPWPLDCTAPSQPCGYFPRCCGHCDVRRVCTSG

SEQ ID NO: 170 - protein, 30 aa, Conus leopardus
  PEPTIDE (1)..(30): Xaa at residue 2, 8, 11 and 16 is Pro or Hyp; Xaa at residue 1 is Trp or Bromo Trp; Xaa at residue 14 is Tyr, 125I-Tyr, mono-iodo-Tyr, di-iodo-Tyr, O-sulpho-Tyr or O-phospho- Tyr
XXLDCTAXSQXCGXFXRCCGHCDVRRVCTS

SEQ ID NO: 171 - DNA, 381 bp, Conus leopardus
atgaaactgacgtgtgtggtgatcgtcgctgtgctgttcctgacggcctgtcaactcactacagctgacatctccagaggtacgcggaagcatcgtgctctgaggtcgaccaccaaactctccaggtcgccctctaggtgcatgtctcccggtggaatttgtggtgattttggtgactgctgcgaaatttgcaatgtgtacggtatatgtgtgagtgacttacccggcatctgatctttccgccttctgtgctctatccttttctgcctgagtcctccatacccctgagtggtcatggaccactcaacacctactcctctggaggcttcagaggaactacattaaaataaagccgcattgcaaaaaaaaaaaaaaaaaaaa

SEQ ID NO: 172 - protein, 77 aa, Conus leopardus
MKLTCVVIVAVLFLTACQLTTADISRGTRKHRALRSTTKLSRSPSRCMSPGGICGDFGDCCEICNVYGICVSDLPGI

SEQ ID NO: 173 - protein, 31 aa, Conus leopardus
  PEPTIDE (1)..(31): Xaa at residue 16 is Glu or gamma-carboxy Glu; Xaa at residue 4 and 29 is Pro or Hyp; Xaa at residue 21 is Tyr, 125I-Tyr, mono-iodo-Tyr, di-iodo-Tyr, O-sulpho-Tyr or O-phospho-Tyr
CMSXGGICGDFGDCCXICNVXGICVSDLXGI

SEQ ID NO: 174 - DNA, 404 bp, Conus leopardus
atgaaactgacgtgtgtggtgatcgtcgctgtgctgttcctgacggcctgtcaactcactacagctgatgattccagaggtacacggaagcatcgtgctctgaggtcaaccaccaaactctccaggtggcccaggtactgcgcgcctcccggtggagcttgtgggttttttgatcactgctgcggatattgcgaaacgttttacaatacgtgtagatgagttggctgatccggcgcttgatctttccgccttctgttgctctatctttttctgcctgagtcctcccataccccgttgagtggtccatgaaccactccaacacctactccctccttggaagcttccaaaggaaacgacatttaaaataaattccccattgcaattggaaaaaaaaaaaaaaaaaa

SEQ ID NO: 175 - protein, 72 aa, Conus leopardus
MKLTCVVIVAVLFLTACQLTTADDSRGTRKHRALRSTTKLSRWPRYCAPPGGACGFFDHCCGYCETFYNTCR

SEQ ID NO: 176 - protein, 27 aa, Conus leopardus
  PEPTIDE (1)..(27): Xaa at residue 20 is Glu or gamma-carboxy Glu; Xaa at residue 4 and 5 is Pro or Hyp; Xaa at residue 1, 18 and 23 is Tyr, 125I-Tyr, mono-iodo-Tyr, di-iodo-Tyr, O-sulpho-Tyr or O- phospho-Tyr
XCAXXGGACGFFDHCCGXCXTFXNTCR

SEQ ID NO: 177 - DNA, 292 bp, Conus lynceus
atgaaactgacgtgtgtggtgatcgtcgccgtgctgctcctgacggcctgtcaactcatcacagctgatgactccagacgtacacagaagcatcgtgccctgaggtcgaccaccaatctctccatgtcgactcgctgcaagtctcccggatcaccatgtagtgtgacatcgtataactgctgcactttttgctcttcatacactaagaaatgtcgggcctctttatgaaccactcatcacctactcctctggaggcctcagaagagctacactgaaataaaagccgcattgg

SEQ ID NO: 178 - protein, 75 aa, Conus lynceus
MKLTCVVIVAVLLLTACQLITADDSRRTQKHRALRSTTNLSMSTRCKSPGSPCSVTSYNCCTFCSSYTKKCRASL

SEQ ID NO: 179 - protein, 30 aa, Conus lynceus
  PEPTIDE (1)..(30): Xaa at residue 4 and 7 is Pro or Hyp; Xaa at residue 13 and 22 is Tyr, 125I-Tyr, mono-iodo-Tyr, di-iodo-Tyr, O-sulpho-Tyr or O-phospho-Tyr
CKSXGSXCSVTSXNCCTFCSSXTKKCRASL

SEQ ID NO: 180 - DNA, 355 bp, Conus lynceus
atgaaactgacgtgtgtggtgatcgtcgccgtgctgctcctgacggcctgtcaactcatcacagctgatgactccagaggtacgcagaagcatcgtgccctgaggtcgaccaccaaactatccatgtatactcgctgcgcaggtccaggagcaatttgtcctaatagggtatgctgcggttattgcagtaaaagaacacatctatgtcattcgcgaactggctgatcttcccccttctgtgctctatcctttttctgcctgagtcctccatacctgagaatggtcatgaaccactcatcacctactcctcttggagacctcagaggagctacactgaaataaaagccgcattggc

SEQ ID NO: 181 - protein, 74 aa, Conus lynceus
MKLTCVVIVAVLLLTACQLITADDSRGTQKHRALRSTTKLSMYTRCAGPGAICPNRVCCGYCSKRTHLCHSRTG

SEQ ID NO: 182 - protein, 28 aa, Conus lynceus
  PEPTIDE (1)..(28): Xaa at residue 4 and 9 is Pro or Hyp; Xaa at residue 16 is Tyr, 125I-Tyr, mono-iodo-Tyr, di-iodo-Tyr, O-sulpho- Tyr or O-phospho-Tyr
CAGXGAICXNRVCCGXCSKRTHLCHSRT

SEQ ID NO: 183 - DNA, 361 bp, Conus lynceus
atgaaactgacgtgtgtggtgatcgtcgccgtgctgctgctagcggcctgtcaactactacacgctgatgactccagaggtacgcagaagactgctgcccgaggtcgaccaccaaaactctccatgctgactcgggcctgctggtcttccggaacaccttgtggtactgatagtttatgctgcggtggatgcaatgtatccaaaagtaaatgtaactagctgattcggcgtctgaacttcccccttctgtgctctatccttttctgcccgagtcctccatacctgagaatggtcatgaaccactcatcacctactcctctggagacctcagaagagctacactgaaataaaagcgcattgc

SEQ ID NO: 184 - protein, 72 aa, Conus lynceus
MKLTCVVIVAVLLLAACQLLHADDSRGTQKTAARGRPPKLSMLTRACWSSGTPCGTDSLCCGGCNVSKSKCN

SEQ ID NO: 185 - protein, 27 aa, Conus lynceus
  PEPTIDE (1)..(27): Xaa at residue 8 is Pro or Hyp; Xaa at residue 3 is Trp or Bromo Trp
ACXSSGTXCGTDSLCCGGCNVSKSKCN

SEQ ID NO: 186 - DNA, 364 bp, Conus lynceus
atgaaactgacgtgtgtggtgatcgtcgccgagctactcctaacggcctgtcaactcatcacagctgatgactccagaggtacgcagaagcatcgtgccctgaggtcgaccaccaatctctccatgctgactcggaagtgctggtctcccggaacctattgtcgtgcgcatagtaaatgctgccgtggatgcgatcagaacagaaataaatgttactagctgattcggcgtctgaacttcctccttctgtgctctatcctttttctgcctgagtcctccatacctgagaatggtcatgaaccactcatcacctactcctctggaggcctcagaggagcctacactgaaataaaagccgcattgg

SEQ ID NO: 187 - protein, 72 aa, Conus lynceus
MKLTCVVIVAELLLTACQLITADDSRGTQKHRALRSTTNLSMLTRKCWSPGTYCRAHSKCCRGCDQNRNKCY

SEQ ID NO: 188 - protein, 27 aa, Conus lynceus
  PEPTIDE (1)..(27): Xaa at residue 5 is Pro or Hyp; Xaa at residue 3 is Trp or Bromo Trp; Xaa at residue 8 and 27 is Tyr, 125I-Tyr, mono-iodo-Tyr, di-iodo-Tyr, O-sulpho-Tyr or O-phospho-Tyr
KCXSXGTXCRAHSKCCRGCDQNRNKCX

SEQ ID NO: 189 - DNA, 318 bp, Conus magus
accaaaaccatcatcaaaatgaaactgacgtgtgtggtgatcgtcgccgtgctgctcctgacggcctgtcaactcatcacagctgatgactccagaggtacgcagaagcatcgtgccctgaggtcggacaccaaactctccatgtcgactcgctgcaagggtacaggaaaaccatgcagtaggattgcgtataactgctgcaccggttcttgcagatcaggtaaatgtggctgatccagtgcctgatcttcccccttctgtgctctatcctttttctgcctgagtcctccttacctgagagtggtcatgaaccactca

SEQ ID NO: 190 - protein, 71 aa, Conus magus
MKLTCVVIVAVLLLTACQLITADDSRGTQKHRALRSDTKLSMSTRCKGTGKPCSRIAYNCCTGSCRSGKCG

SEQ ID NO: 191 - protein, 25 aa, Conus magus
  PEPTIDE (1)..(25): Xaa at residue 7 is Pro or Hyp; Xaa at residue 13 is Tyr, 125I-Tyr, mono-iodo-Tyr, di-iodo-Tyr, O-sulpho-Tyr or O-phospho-Tyr
CKGTGKXCSRIAXNCCTGSCRSGKC

SEQ ID NO: 192 - DNA, 259 bp, Conus magus
accaaaaccatcatcaaaatgaaactgacgtgtgtggtgatcgtcgccgtgctgctcctgacggcctgtcaactcatcacagctgatgactccagaggtacgcagaagcatcgtgccctgaagtcggacaccaaactctccatgttaactttgcgctgcgcatcttacggaaaaccttgtggtatttacaacgactgctgcaatacatgcgatccagccagaaagacatgtacgtagctgatccggcgtctgatcttcc

SEQ ID NO: 193 - protein, 72 aa, Conus magus; identical to SEQ ID NO: 115

SEQ ID NO: 194 - protein, 26 aa, Conus magus
  PEPTIDE (1)..(26): Xaa at residue 7 and 20 is Pro or Hyp; Xaa at residue 4 and 11 is Tyr, 125I-Tyr, mono-iodo-Tyr, di-iodo-Tyr, O- sulpho-Tyr or O-phospho-Tyr
CASXGKXCGIXNDCCNTCDXARKTCT

SEQ ID NO: 195 - DNA, 254 bp, Conus magus
gaattttcagcatcaccaaaaccatcatcaaaatgaaactgacgtgtgtggtgatcgtcgccgtgctgctcctgacggcctgtcaactcatcacagctgatgactccagaggtacgcagaagcatcgtgccctgaggtcggacaccaaactctccatgtcaactcgctgcaagggtaaaggagcatcatgtcataggacttcgtatgactgctgcaccggttcttgcaacagaggtaaatttggctgatccgcc

SEQ ID NO: 196 - protein, 71 aa, Conus magus
MKLTCVVIVAVLLLTACQLITADDSRGTQKHRALRSDTKLSMSTRCKGKGASCHRTSYDCCTGSCNRGKFG

SEQ ID NO: 197 - protein, 25 aa, Conus magus
  PEPTIDE (1)..(25): Xaa at residue 13 is Tyr, 125I-Tyr, mono-iodo- Tyr, di-iodo-Tyr, O-sulpho-Tyr or O-phospho-Tyr
CKGKGASCHRTSXDCCTGSCNRGKC

SEQ ID NO: 198 - DNA, 358 bp, Conus miles
ggatccatgaaactgacgtgcgtggtgatcatcgccatgctgttcctgacagcctatcaactcgctacagctgcgagctacgccaaaggtaaacagaagcatcgtgctctgaggccagctgacaaacacctcaggttgaccaagcgttgcaatgatcgcggtggaggttgcagtcaacatcctcactgctgcggtggaacttgcaataagcttattggcgtatgtctgtaaagctggtctgccgtctgatattccctttctgtgcttcatcctcttttgcctgagtcatccatacctgtgaatggttaagagccactcaatacctattcctctgggggcttcagaggaactactttac

SEQ ID NO: 199 - protein, 74 aa, Conus miles
MKLTCVVIIAMLFLTAYQLATAASYAKGKQKHRALRPADKHLRLTKRCNDRGGGCSQHPHCCGGTCNKLIGVCL

SEQ ID NO: 200 - protein, 27 aa, Conus arenatus
  PEPTIDE (1)..(27): Xaa at residue 12 is Pro or Hyp
CNDRGGGCSQHXHCCGGTCNKLIGVCL

SEQ ID NO: 201 - DNA, 292 bp, Conus monachus
accaaaaccatcatcaaaatgaaactgacgagtgtggtgatcgtcgccgtgctgctcctgacggcctgtcaactcatcacagctgatgactccagaggtacgcagaagcatcgtgccctgaggtcggacaccaaactctccatatcgactcgctgcaagtctacaggaaaatcatgcagtaggattgcgtataactgctgcaccggttcttgcagatcaggtaaatgtggctgatccagcgcctgatcttcccccttctgtgctctatccttttctgcctgagtcctcctta

SEQ ID NO: 202 - protein, 71 aa, Conus monachus
MKLTSVVIVAVLLLTACQLITADDSRGTQKHRALRSDTKLSISTRCKSTGKSCSRIAYNCCTGSCRSGKCG

SEQ ID NO: 203 - protein, 25 aa, Conus monachus
  PEPTIDE (1)..(25): Xaa at residue 13 is Tyr, 125I-Tyr, mono-iodo- Tyr, di-iodo-Tyr, O-sulpho-Tyr or O-phospho-Tyr
CKSTGKSCSRIAXNCCTGSCRSGKC

SEQ ID NO: 204 - DNA, 258 bp, Conus monachus
accaaaaccatcatcaaaatgaaactgacgagtgtggtgatcgtcgccgtgctgctcctgacggcctgtcaactcatcacagctgatgactccagaggtacgcagaagcatcgtgccctgaggtcggacaccaacctctccatgtcgactcgctgcaagggtaaaggatcttcatgtagtaggaccatgtataactgctgcaccggttcttgcaacagaggtaaatgtggctgatccagcgcctgatcttcccccttc

SEQ ID NO: 205 - protein, 71 aa, Conus monachus
MKLTSVVIVAVLLLTACQLITADDSRGTQKHRALRSDTNLSMSTRCKGKGSSCSRTMYNCCTGSCNRGKCG

SEQ ID NO: 206 - protein, 25 aa, Conus monachus
  PEPTIDE (1)..(25): Xaa at residue 13 is Tyr, 125I-Tyr, mono-iodo- Tyr, di-iodo-Tyr, O-sulpho-Tyr or O-phospho-Tyr
CKGKGSSCSRTMXNCCTGSCNRGKC

SEQ ID NO: 207 - DNA, 258 bp, Conus obscurus
ctctctctctctctgctggacaggtcgcctccctgcatgaaaggcggatcgtcatgccgcggtactacgggagtctgttgcggtttttgcagtgatttcggctataaatgtagggactatccccaaaactgatcttcccccttctgtgctctatccttttctgtccgagtcctcctgacctgagagtggtcatgaaccactcatcacctacccctctggggcttcacaggatctacattgaaataaaagccgcattgc

SEQ ID NO: 208 - protein, 39 aa, Conus obscurus
LLDRSPPCMKGGSSCRGTTGVCCGFCSDFGYKCRDYPQN

SEQ ID NO: 209 - protein, 35 aa, Conus obscurus
  PEPTIDE (1)..(35): Xaa at residue 2, 3 and 33 is Pro or Hyp; Xaa at residue 27 and 32 is Tyr, 125I-Tyr, mono-iodo-Tyr, di-iodo- Tyr, O-sulpho-Tyr or O-phospho-Tyr
SXXCMKGGSSCRGTTGVCCGFCSDFGXKCRDXXQN

SEQ ID NO: 210 - DNA, 259 bp, Conus obscurus
ctctctctctctctgctggacaggtcgactcgctgcttgcctgacggaacgtcttgcctttttagtaggatcagatgctgcggtacttgcagttcaatcttaaagtcatgtgtgagctgatccagcggttgatcttcctccctctgtgctccatccttttctgcctgagttctccttacctgagagtggtcatgaaccactcatcacctactcttctggaggcttcagaggagctacattgaaataaaagccgcattgc

SEQ ID NO: 211 - protein, 32 aa, Conus obscurus
RSTRCLPDGTSCLFSRIRCCGTCSSILKSCVS

SEQ ID NO: 212 - protein, 28 aa, Conus monachus
  PEPTIDE (1)..(28): Xaa at residue 3 is Pro or Hyp
CLXDGTSCLFSRIRCCGTCSSILKSCVS

SEQ ID NO: 213 - DNA, 330 bp, Conus pulicarius
  misc_feature (1)..(330): n may be any nucleotide
atgaaactgacgtgtgtggtgatcatcgccgtgctgttcctgacggcctgtcaactcattacagctgagacttactccagaggtaagcagaagcaccgtgctttgaggtcaactgacaaaaactccaagttgactaggcagtgctcgcctaacggtggatcttgttctcgtcattttcactgctgcagcctctattgcaataaaaatactggcgtatgtattgcaacctaatacccgtgtgtggtcatgaaccactcaataccctctcctctggaggcttcagaggaactgcattgaaataaaactgcattgcnttgaccaaaaaaaaaa

SEQ ID NO: 214 - protein, 76 aa, Conus pulicarius
MKLTCVVIIAVLFLTACQLITAETYSRGKQKHRALRSTDKNSKLTRQCSPNGGSCSRHFHCCSLYCNKNTGVCIAT

SEQ ID NO: 215 - protein, 30 aa, Conus pulicarius
  PEPTIDE (1)..(30): Xaa at residue 1 is Gln or pyro-Glu; Xaa at residue 4 is Pro or Hyp; Xaa at residue 19 is Tyr, 125I-Tyr, mono-iodo-Tyr, di-iodo-Tyr, O-sulpho-Tyr or O-phospho-Tyr
XCSXNGGSCSRHFHCCSLXCNKNTGVCIAT

SEQ ID NO: 216 - DNA, 282 bp, Conus purpurascens
atgaaactgacgtgtgtggtgatcgtcgccgtgctgttcctgacggcctgtcaactcatcacagctgatgactccagacgtacgcagaagcatcgtgccctgaggtcgaccaccaaaggcgccacgtcgaatcgcccctgcaagacacccggacgaaaatgttttccgcatcagaaggactgctgcggtcgagcgtgcatcatcacaatatgtccctgatcttcccccttctgtgctgtatccttttctgcctgagtctccttacctgagagtggtcatgaa

SEQ ID NO: 217 - protein, 72 aa, Conus purpurascens
MKLTCVVIVAVLFLTACQLITADDSRRTQKHRALRSTTKGATSNRPCKTPGRKCFPHQKDCCGRACIITICP

SEQ ID NO: 218 - protein, 27 aa, Conus purpurascens
  PEPTIDE (1)..(27): Xaa at residue 1, 5, 11 and 27 is Pro or Hyp
XCKTXGRKCFXHQKDCCGRACIITICX

SEQ ID NO: 219 - DNA, 340 bp, Conus purpurascens
accaaaaccatcatcaaaatgaaactgacgtgtgtggtgatcgtcgccgtgctgctcctgacggcctgtcaactcatcacagctgatgactccagaggtacgcagaagcatcgtgccctgaggtcgaccaccaaactcttcacgtcgaaaagctgcaagcttcccggagcatattgtaatgcagaagattatgactgctgccttagatgcaaagttggaggtacatgtggctgatccagtgcctgatcttcccccttctgtgctctatccttttctgcctgagtcctccttacctaagagtggtcatgaaccactcatcaccttctcctctggaggcttc

SEQ ID NO: 220 - protein, 71 aa, Conus purpurascens
MKLTCVVIVAVLLLTACQLITADDSRGTQKHRALRSTTKLFTSKSCKLPGAYCNAEDYDCCLRCKVGGTCG

SEQ ID NO: 221 - protein, 26 aa, Conus purpurascens
  PEPTIDE (1)..(26): Xaa at residue 12 is Glu or gamma-carboxy Glu; Xaa at residue 5 is Pro or Hyp; Xaa at residue 8 and 14 is Tyr, 125I-Tyr, mono-iodo-Tyr, di-iodo-Tyr, O-sulpho-Tyr or O-phospho- Tyr
SCKLXGAXCNAXDXDCCLRCKVGGTC

SEQ ID NO: 222 - DNA, 317 bp, Conus purpurascens
atgaaactgacgtgtgtggtgatcgtcgccgtgctgttcctgacggcctgtcaactcatcacagctgatgactccagacgtacgcagaagcatcgtgccctgaggtcgaccaccaaacgcgccacgtcgaatcgcccctgcaagaaaaccggacgaaaatgttttccgcatcagaaggactgctgcggtcgagcgtgcatcatcacaatatgtccctgatcttcccccttctgtgctgtatccttttctgcctgagtcctccttacctgagagtggtcatgaaccactcatcaccttctcctctggaggcttcagag

SEQ ID NO: 223 - protein, 72 aa, Conus purpurascens
MKLTCVVIVAVLFLTACQLITADDSRRTQKHRALRSTTKRATSNRPCKKTGRKCFPHQKDCCGRACIITICP

SEQ ID NO: 224 - protein, 27 aa, Conus purpurascens
  PEPTIDE (1)..(27): Xaa at residue 1, 11 and 27 is Pro or Hyp
XCKKTGRKCFXHQKDCCGRACIITICX

SEQ ID NO: 225 - DNA, 328 bp, Conus radiatus
gctgatgcctgatcttcatcgttcttccctgtctcctttggcatcaccaaaaccatcatcaaaatgaaactgacgtgtgtggtgatcgtcgccgtgctggtcctgacggcctgtcaactcatcacagctgatgactccagaggtatgcagaaacatcatgccctggggtcgatcagcagtctctttaagtcgacccgtcatggctgcaaacccctcaaacgtcgttgtttcaatgataaagaatgctgcagcaaattttgcaattcagtccgaaagcagtgtggataaatggctaaaaaactgaataaaagccgcattgcaaaaaaaa

SEQ ID NO: 226 - protein, 74 aa, Conus radiatus
MKLTCVVIVAVLVLTACQLITADDSRGMQKHHALGSISSLFKSTRHGCKPLKRRCFNDKECCSKFCNSVRKQCG

SEQ ID NO: 227 - protein, 28 aa, Conus radiatus
  PEPTIDE (1)..(28): Xaa at residue 15 is Glu or gamma-carboxy Glu; Xaa at residue 5 is Pro or Hyp
HGCKXLKRRCFNDKXCCSKFCNSVRKQC

SEQ ID NO: 228 - DNA, 250 bp, Conus radiatus
gaaatgaaactgacgtgtgtggtgatcgtcgccgtgctggtcctgacggcctgtcaactcatcacagctgatgactccagaggtatgcagaaacatcatgccctggggtcgatcagcagtctctttaagtcgacccgtcgtggctgcaaacccctcaaacgtcgttgtttcaatgataaagaatgctgcagcaaattttgcaattcagtccgaaaccagtgtggataaatggctaaaaactgaataaaag

SEQ ID NO: 229 - protein, 74 aa, Conus radiatus
MKLTCVVIVAVLVLTACQLITADDSRGMQKHHALGSISSLFKSTRRGCKPLKRRCFNDKECCSKFCNSVRNQCG

SEQ ID NO: 230 - protein, 28 aa, Conus radiatus
  PEPTIDE (1)..(28): Xaa at residue 15 is Glu or gamma-carboxy Glu; Xaa at residue 5 is Pro or Hyp
RGCKXLKRRCFNDKXCCSKFCNSVRNQC

SEQ ID NO: 231 - DNA, 435 bp, Conus radiatus
ggaattccgcttgcacggcgaacctgacttcatctttcttccctgcctcctttggcatcaccaaaaccatcatcaaaatgaaactgacgtgtgtggtgatcgtcgccgtgctggtcctgacggcctgtcaactcatcacagctgatgactccagaggtatgcagaagcatcatgccctgaggtcgatcaccaaactctccctgtcgactcgctgcaaacctcccggatcaccatgtagagtttcttcgtataactgctgctcttcttgcaaatcatacaacaagaaatgtggctgaacttccccttctgtgctctatccttttcctgcccgagtcctccatacctgagagtagtcatgaaccactgattacctactcctctggagggcctcagaggagctactttgaaataaaagcccgcattgcaaaaaaaaaa

SEQ ID NO: 232 - protein, 72 aa, Conus radiatus
MKLTCVVIVAVLVLTACQLITADDSRGMQKHHALRSITKLSLSTRCKPPGSPCRVSSYNCCSSCKSYNKKCG

SEQ ID NO: 233 - protein, 27 aa, Conus radiatus
  PEPTIDE (1)..(27): Xaa at residue 3, 4 and 7 is Pro or Hyp; Xaa at residue 13 and 22 is Tyr, 125I-Tyr, mono-iodo-Tyr, di-iodo- Tyr, O-sulpho-Tyr or O-phospho-Tyr
CKXXGSXCRVSSXNCCSSCKSXNKKCG

SEQ ID NO: 234 - DNA, 392 bp, Conus rattus
ggatccatgaaactgacgtgcatggtgatcatcgccgtgctgttcctgacagcctgtcaattcgatacagctgcgagctacgacaaaggtaagcagaaacctcctactctgaggccagctgacaaacacatcaggttgaccaagcgttgcaatgctcgcaatgatggttgcagtcaacattctcaatgctgcagtggatcttgcaataagactgcaggcgtatgtctgtaaagctggtctgccgtctgatattccctttctgtgctttatcctcttttgcctgagtcatccatacctgtgaatggttaagagccactcaatacctactcctctgggggcttcagaggaactacattaaataaagccacattgcaaaaaaaaaaaaaaaaaaa

SEQ ID NO: 235 - protein, 74 aa, Conus rattus
MKLTCMVIIAVLFLTACQFDTAASYDKGKQKPPTLRPADKHIRLTKRCNARNDGCSQHSQCCSGSCNKTAGVCL

SEQ ID NO: 236 - protein, 27 aa, Conus rattus
CNARNDGCSQHSQCCSGSCNKTAGVCL

SEQ ID NO: 237 - DNA, 395 bp, Conus rattus
ggatccatgaaactgacgtgcgtggtgatcatcgccgtgctgttcctgacagcctgtcaactcgatgcagctgcgagctacgacaaaggtaagcagaaacctcctactctgaggccagctgacaaacacttcaggttgatcaagcgttgcaatgctcgcaatagtggttgcagtcaacatcctcaatgctgcagtggatcttgcaataagactgcaggcgtatgtctgtaaagctggtctgccgtctgatattccctttctgtgctttatcctcttttgcctgagtcatccatacctgtgaatggttaagagccactcaatacctactcctctgggggcttcagaggaactacattaaataaagccacattgcaacgaaaaaaaaaaaaaaaaaa

SEQ ID NO: 238 - protein, 74 aa, Conus rattus
MKLTCVVIIAVLFLTACQLDAAASYDKGKQKPPTLRPADKHFRLIKRCNARNSGCSQHPQCCSGSCNKTAGVCL

SEQ ID NO: 239 - protein, 27 aa, Conus rattus
  PEPTIDE (1)..(27): Xaa at residue 12 is Pro or Hyp
CNARNSGCSQHXQCCSGSCNKTAGVCL

SEQ ID NO: 240 - DNA, 390 bp, Conus rattus
ggatccatgaaactgacgtgtgtggtgatcatcgccgtgctgttcctgacagcctgtcaattcgatacagctgcgagctacgacaaaggtaagcagaaacctcctactctgaggccagctgacaaacacttcaggttgatcaagcgttgcaatgctcgcaatagtggttgcagtcaacatcctcaatgctgcagtggatcttgcaataagactttgggcgtatgtctgtaaagctggtctgccgtctgatattccctttctgtgctttatcctcttttgcctgagtcatccatacctgtgaatggttaagagccactcaatacctactcctctgggggcttcagaggaactacattaaataaagccacattgaaaaaaaaaaaaaaaaaa

SEQ ID NO: 241 - protein, 74 aa, Conus rattus
MKLTCVVIIAVLFLTACQFDTAASYDKGKQKPPTLRPADKHFRLIKRCNARNSGCSQHPQCCSGSCNKTLGVCL

SEQ ID NO: 242 - protein, 27 aa, Conus rattus
  PEPTIDE (1)..(27): Xaa at residue 12 is Pro or Hyp
CNARNSGCSQHXQCCSGSCNKTLGVCL

SEQ ID NO: 243 - DNA, 379 bp, Conus stercusmuscarum
accaaaaccatcatcaaaatgaaactgacgtgtgtggtgatcgtcgccgtgctgctcctgacggcctgtcaactcatcacagctgatgactccagaggtacgcagaagcatcgtgccctgaggtcgaagaccaaactctccatgtcgactcgctgcaagagtaaaggagcaaaatgttcaaggcttatgtatgactgctgcagcggttcttgcagcggctacacaggtagatgtggctgatccagcgcctgatcttcccccttctgtgctctatccttttctgcctgggtcctccttacctgagagtggtcatgaaccactcatcacctactcctctggaggcctcagaggagttacaatgaaataaaagccgcattgc

SEQ ID NO: 244 - protein, 73 aa, Conus stercusmuscarum
MKLTCVVIVAVLLLTACQLITADDSRGTQKHRALRSKTKLSMSTRCKSKGAKCSRLMYDCCSGSCSGYTGRCG

SEQ ID NO: 245 - protein, 27 aa, Conus stercusmuscarum
  PEPTIDE (1)..(27): Xaa at residue 13 and 23 is Tyr, 125I-Tyr, mono-iodo-Tyr, di-iodo-Tyr, O-sulpho-Tyr or O-phospho-Tyr
CKSKGAKCSRLMXDCCSGSCSGXTGRC

SEQ ID NO: 246 - protein, 35 aa, Conus stercusmuscarum
  PEPTIDE (1)..(35): Xaa at residue 33 is Pro or Hyp; Xaa at residue 10, 21, 24 and 32 is Tyr, 125I-Tyr, mono-iodo-Tyr, di-iodo-Tyr, O-sulpho-Tyr or O-phospho-Tyr
TTSCMQAGSXCGSTTRICCGXCAXFGKKCIDXXSN

SEQ ID NO: 247 - DNA, 380 bp, Conus stercusmuscarum
accaaaaccatcatcaaaatgaaactgacgtgtgtggtgatcgtcgccgtgctgctcctgacgacctgtcaactcatcacagctgatgactccagaggtacgcaggagcatcgtgccctgaggtcgaagaccaaactctccatgttaactttgcgctgcgcatcttacggaaaaccttgtggtattgacaacgactgctgcaatgcatgcgatccagccagaaatatatgtacgtagctgatccggcgtctgatcttcccccttctgtgctctatccttttctgcctgagtcctccttacctgagagtggtcatgaaccactcatcatctactctcctggaggcctcagaggagctacaatgaaataaaagccgcattgc

SEQ ID NO: 248 - protein, 72 aa, Conus stercusmuscarum
MKLTCVVIVAVLLLTTCQLITADDSRGTQEHRALRSKTKLSMLTLRCASYGKPCGIDNDCCNACDPARNICT

SEQ ID NO: 249 - protein, 26 aa, Conus stercusmuscarum
  PEPTIDE (1)..(26): Xaa at residue 7 and 20 is Pro or Hyp; Xaa at residue4  is Tyr, 125I-Tyr, mono-iodo-Tyr, di-iodo- Tyr, O-sulpho-Tyr or O-phospho-Tyr
CASXGKXCGIDNDCCNACDXARNICT

SEQ ID NO: 250 - DNA, 388 bp, Conus stercusmuscarum
ggatccatgaaactgacgtgtgtggtgattgtcgccgtgctgctcctgacggcctgtcaactcatcacagctgatgactccagaggtacgcaggagcatcgtgccctgaggtcgaagaccaaactctccatgttaactttgcgctgcgtatcttacggaaaaccttgtggtattgacaacgactgctgcaatgcatgcgatccagccagaaatatatgtacgtagctgatccggcgtctgatcttcccccttctgtgctctatccttttctgcctgggtcctccttacctgagagtggtcatgaaccactcatcacctactcctctggaggcctcagaggagttacaatgaaataaaagccgcattgcaaaaaaaaaaaaaaaaaaaa

SEQ ID NO: 251 - protein, 72 aa, Conus stercusmuscarum
MKLTCVVIVAVLLLTACQLITADDSRGTQEHRALRSKTKLSMLTLRCVSYGKPCGIDNDCCNACDPARNICT

SEQ ID NO: 252 - protein, 26 aa, Conus stercusmuscarum
  PEPTIDE (1)..(26): Xaa at residue 7 and 20 is Pro or Hyp; Xaa at residue 4 is Tyr, 125I-Tyr, mono-iodo-Tyr, di-iodo- Tyr, O-sulpho-Tyr or O-phospho-Tyr
CVSXGKXCGIDNDCCNACDXARNICT

SEQ ID NO: 253 - DNA, 264 bp, Conus striatus
accaaaaccatcatcaaaatgaaactgacgtgtgtggtgatcgtcgccgtgctgctcctgacggcctgtcaactcatcacagctgatgactccagaggtacgcagaagcatcgttccctgaggtcgaccaccaaagtctccaaggcgactgactgcattgaagccggaaattattgcggacctactgttatgaaaatctgctgcggcttttgcagtccatacagcaaaatatgtatgaactatcccaaaaattgatcttccccc

SEQ ID NO: 254 - protein, 78 aa, Conus striatus
MKLTCVVIVAVLLLTACQLITADDSRGTQKHRSLRSTTKVSKATDCIEAGNYCGPTVMKICCGFCSPYSKICMNYPKN

SEQ ID NO: 255 - protein, 36 aa, Conus striatus
  PEPTIDE (1)..(36): Xaa at residue 6 is Glu or gamma-carboxy Glu; Xaa at residue 13,25 and 34 is Pro or Hyp; Xaa at residue 10, 26 and 33 is Tyr, 125 I-Tyr, mono-iodo-Tyr, di-iodo-Tyr, O-sulpho-Tyr or O-phospho-Tyr
ATDCIXAGNXCGXTVMKICCGFCSXXSKICMNXXKN

SEQ ID NO: 256 - DNA, 233 bp, Conus striatus
gtcgactcgctgcaagcttaaaggacaatcatgtcgtaggactatgtatgactgctgcagcggttcttgcggcaggagaggtaaatgtggctgatccagcgcctgatctccccccttctgtgctctatccttttctgcctgggtcctccttacctgagagtggtcatgaaccactcatcacctactcctctggaggcctcagaggagctacaatgaaataaaagccgcattgc

SEQ ID NO: 257 - protein, 30 aa, Conus striatus
STRCKLKGQSCRRTMYDCCSGSCGRRGKCG

SEQ ID NO: 258 - protein, 26 aa, Conus striatus
  PEPTIDE (1)..(26): Xaa at residue 13 is Tyr, 125I-Tyr, mono-iodo-Tyr, di-iodo-Tyr, O-sulpho-Tyr or O-phospho-Tyr
CKLKGQSCRRTMXDCCSGSCGRRGKC

SEQ ID NO: 259 - DNA, 310 bp, Conus striatus
accaaaaccatcatcaaaatgaaactgacgtgtgtggtgatcgtcgccgtgctgctcctgacggcctgtcaactcatcacagctgatgactccagaggtacgcagaagcatcgtgccctgaggtcggacaccaaactctccatgtcgactcgctgcaaggctgcaggaaaatcatgcagtaggattgcgtataactgctgcaccggttcttgcagatcaggtaaatgcggctgatccagcgcctgatcttcccccttctgtgctctatcctttctgcctgagtcctcttacctgagagtggtcatgaacc

SEQ ID NO: 260 - protein, 71 aa, Conus striatus
MKLTCVVIVAVLLLTACQLITADDSRGTQKHRALRSDTKLSMSTRCKAAGKSCSRIAYNCCTGSCRSGKCG

SEQ ID NO: 261 - protein, 25 aa, Conus striatus
  PEPTIDE (1)..(25): Xaa at residue 13 is Tyr, 125I-Tyr, mono-iodo-Tyr, di-iodo-Tyr, O-sulpho-Tyr or O-phospho-Tyr
CKAAGKSCSRIAXNCCTGSCRSGKC

SEQ ID NO: 262 - DNA, 256 bp, Conus striatus
accaaaaccatcatcaaaatgaaactgacgtgtgtggtgatcgtcgccgtgctgctcctgacggcctgtcaactcatcacagctgatgactccagaggtacgcaggagcatcgtgccctgaggtcggacaccaaactctccatgttaactttgcgctgcgaatcttacggaaaaccttgtggtatttacaacgactgctgcaatgcatgcgatccagccaaaaagacatgtacgtagctgatccggcgtctgatct

SEQ ID NO: 263 - protein, 72 aa, Conus striatus
MKLTCVVIVAVLLLTACQLITADDSRGTQEHRALRSDTKLSMLTLRCESYGKPCGIYNDCCNACDPAKKTCT

SEQ ID NO: 264 - protein, 26 aa, Conus striatus
  PEPTIDE (1)..(26): Xaa at residue 2 is Glu or gamma-carboxy Glu; Xaa at residue 7 and 20 is Pro or Hyp; Xaa at residue 4 and 11 is Tyr, 125I-Tyr, mono-iodo-Tyr, di-iodo-Tyr, O-sulpho-Tyr or O-phospho-Tyr
CXSXGKXCGIXNDCCNACDXAKKTCT

SEQ ID NO: 265 - DNA, 229 bp, Conus striatus
tctaggtcctccggcagcccctgtggtgttactagtatatgctgtggtagatgctataggggtaaatgtacgtagctcatcgggcgtctgatcttcccccttctgtgctccatccttttctgcctgagtcctccttacctgagagtggtcgtgaaccactcatcgcctactcctctggaggcttcagaggggctacactaaaataaaagctatattgcaatgaaaaaaa

SEQ ID NO: 266 - protein, 24 aa, Conus striatus
CRSSGSPCGVTSICCGRCYRGKCT

SEQ ID NO: 267 - protein, 24 aa, Conus striatus
  PEPTIDE (1)..(24): Xaa at residue 7 is Pro or Hyp; Xaa at residue 19 is Tyr, 125I-Tyr, mono-iodo-Tyr, di-iodo-Tyr, O-sulpho-Tyr or O-phospho-Tyr
CRSSGSXCGVTSICCGRCXRGKCT

SEQ ID NO: 268 - protein, 26 aa, Conus striatus
  PEPTIDE (1)..(26): Xaa at residue 13 is Tyr, 125I-Tyr, mono- iodo-Tyr, di-iodo-Tyr, O-sulpho-Tyr or O-phospho-Tyr
CKLKGQSCRKTSXDCCSGSCGRSGKC

SEQ ID NO: 269 - DNA, 292 bp, Conus striolatus
accaaaaccatcatcaaaatgaaactgacgtgtgtggtgatcgtcgtcttgctgctcctgacgacctgtcgtctcatcacagctgatgactccagaggtacgcagaagcatcgttccctgaggtcgactactaaagtctccatgtcgactcgctgcaagggtaaaggagcatcatgtcttaggactgcgtatgactgctgcaccggttcttgcaacagaggtagatgtggctgatccagcgtctgatcttcccccttctgtgctctatccttttctgcttgagtcctcctta

SEQ ID NO: 270 - protein, 71 aa, Conus striolatus
MKLTCVVIVVLLLLTTCRLITADDSRGTQKHRSLRSTTKVSMSTRCKGKGASCLRTAYDCCTGSCNRGRCG

SEQ ID NO: 271 - protein, 25 aa, Conus striolatus
  PEPTIDE (1)..(25): Xaa at residue 13 is Tyr, 125I-Tyr, mono- iodo-Tyr, di-iodo-Tyr, O-sulpho-Tyr or O-phospho-Tyr
CKGKGASCLRTAXDCCTGSCNRGRC

SEQ ID NO: 272 - DNA, 259 bp, Conus striolatus
accaaaaccatcatcaaaatgaaactgacgtgtgtggtgatcgtcgccgttctgctgacggcgtgtcaactcatcacagctgaggactccagaggtacacagaagcatcgtaccctgaggtcgaccgtcagacgctccaagtccgagttgactacgagatgcaggccttcaggatccaactgtggtaatattagtatctgctgtggtagatgcgttaacagaagatgtacgtagctcatcgggcgtctgatctttcccc

SEQ ID NO: 273 - protein, 71 aa, Conus striolatus
MKLTCVVIVAVLLTACQLITAEDSRGTQKHRTLRSTVRRSKSELTTRCRPSGSNCGNISICCGRCVNRRCT

SEQ ID NO: 274 - protein, 24 aa, Conus striolatus
  PEPTIDE (1)..(24): Xaa at residue 3 is Pro or Hyp
CRXSGSNCGNISICCGRCVNRRCT

SEQ ID NO: 275 - DNA, 280 bp, Conus striolatus
accaaaaccatcatcaaaatgaaactgacgtgtgtggtgatcgtcgccgttctgttcctgacggcgtgtcaactcatcacagctgaggactccagaggtacacagaagcatcgttccctgaggtcgactaccaaagtctccaagtcgactagctgcatgaaagccgggtcttattgcgtcgctactacgagaatctgctgcggttattgcgcttatttcggcaaaatatgtattgactatcccaaaaactgatcttccccctactgtgctctatcctttt

SEQ ID NO: 276 - protein, 77 aa, Conus striolatus
MKLTCVVIVAVLFLTACQLITAEDSRGTQKHRSLRSTTKVSKSTSCMKAGSYCVATTRICCGYCAYFGKICIDYPKN

SEQ ID NO: 277 - protein, 35 aa, Conus striolatus
  PEPTIDE (1)..(35): Xaa at residue 33 is Pro or Hyp; Xaa at residue 10, 21, 24 and 32 is Tyr, 125I-Tyr, mono-iodo-Tyr, di-iodo-Tyr, O-sulpho-Tyr or O-phospho-Tyr
STSCMKAGSXCVATTRICCGXCAXFGKICIDXXKN

SEQ ID NO: 278 - DNA, 174 bp, Conus textile
gttgactcggtactgcacgcctcatggaggacattgtggttatcataatgactgctgcagtcatcaatgcaatataaacagaaataaatgtgagtagctgatctggcatctgatctgtgctcgtccttacctgagagtggtcatgaaccactcatcacctactcctctggaggc

SEQ ID NO: 279 - protein, 31 aa, Conus textile
LTRYCTPHGGHCGYHNDCCSHQCNINRNKCE

SEQ ID NO: 280 - protein, 28 aa, Conus textile
  PEPTIDE (1)..(28): Xaa at residue 28 is Glu or gamma-carboxy Glu; Xaa at residue 4 is Pro or Hyp; Xaa at residue 1 and 11 is Tyr, 125I-Tyr, mono-iodo-Tyr, di-iodo-Tyr, O-sulpho-Tyr or O-phospho- Tyr
XCTXHGGHCGXHNDCCSHQCNINRNKCX

SEQ ID NO: 281 - protein, 28 aa, Conus textile
  PEPTIDE (1)..(28): Xaa at residue 28 is Glu or gamma-carboxy Glu; Xaa at residue 4 is Pro or Hyp; Xaa at residue 1 and 11 is Tyr, 1 -Tyr, di-iodo-Tyr, O-sulpho-Tyr or O-phospho-Tyr
XCTXXGGHCGXHNDCCSHQCNINRNKCX

SEQ ID NO: 282 - DNA, 379 bp, Conus tulipa
accaaaaccatcatcaaaatgaaactgacgtgtgtggtgatcgtcgccgtgctgctcctgacggcctgtcagctcatcacagctctgcactccagaggtacgcagaagcatcgtgccctggggcggaccaccaaactcaccttgtcgactcgctgcaaatcacccggatctccatgttcaccgactagttataattgctgctggtcttgcagtccatacagaaaaaaatgtaggggctaatccagcgcctgattttcccccttctgtgctctattcctttctgcctgagtcctccttacctgaaagtggtcatgaaccactcatcacctacttctctggaggcttcggaggagctacattgaaataaaagccgcattgc

SEQ ID NO: 283 - protein, 73 aa, Conus tulipa
MKLTCVVIVAVLLLTACQLITALHSRGTQKHRALGRTTKLTLSTRCKSPGSPCSPTSYNCCWSCSPYRKKCRG

SEQ ID NO: 284 - protein, 27 aa, Conus tulipa
  PEPTIDE (1)..(27): Xaa at residue 3, 7, 10 and 21 is Pro or Hyp; Xaa at residue 17 is Trp or Bromo Trp; Xaa at residue 13 and 22 is Tyr, 125I-Tyr, mono-iodo-Tyr, di-iodo-Tyr, O-sulpho-Tyr or O-phospho-Tyr
CKSXGSXCSXTSXNCCXSCSXXRKKCR

SEQ ID NO: 285 - DNA, 379 bp, Conus tulipa
accaaaaccatcatcaaaatgaaactgacgtgtgtggtgatcgtcgccgtgctgctcctgacggcctgtcagctcatcacagctctgcactccagaggtacgcagaagcatcgtgccctggggtcgaccaccaaactcaccttgtcgactcgctgcttgtcacccggatcttcatgttcaccgactagttataattgctgcaggtcttgcaatccatacagcagaaaatgtaggggctaatccagcgcctgatcttcccccttctgtgctctattcctttctgcctgagtcctccttacctgaaagtggtcatgaaccactcatcacctacttctctggaggcttcggaggagctacattgaaataaaagccgcattgc

SEQ ID NO: 286 - protein, 73 aa, Conus tulipa
MKLTCVVIVAVLLLTACQLITALHSRGTQKHRALGSTTKLTLSTRCLSPGSSCSPTSYNCCRSCNPYSRKCRG

SEQ ID NO: 287 - protein, 27 aa, Conus tulipa
  PEPTIDE (1)..(27): Xaa at residue 4, 10 and 21 is Pro or Hyp; Xaa at residue 13 and 22 is Tyr, 125I-Tyr, mono-iodo-Tyr, di- iodo-Tyr, O-sulpho-Tyr or O-phospho-Tyr
CLSXGSSCSXTSXNCCRSCNXXSRKCR

SEQ ID NO: 288 - DNA, 401 bp, Conus viola
accaaaaccatcatcaaaatgaaactgacgtgtgtggtgatcgtcgccgtgctgctcctgacggcctgtcagctcattacagctgatgactccagaggtacgcagttgcatcgtgccctgaggaaggccaccaaactccccgtgtcgactcgctgcattactttaggaacacgatgtaaggttccgagtcaatgctgcagatcttcttgcaagaacggtcgttgtgctccatcccctgaagaatggtaaatgtggctgatccagcgcctgatcttcccccttctgactgtctccgaccttttctgcctgagtcctccttacctgagaggtgtcatgaaccactcatcacctactcccctggaagcttcagaggagctacattgaaataaaagccgcattgc

SEQ ID NO: 289 - protein, 76 aa, Conus viola
MKLTCVVIVAVLLLTACQLITADDSRGTQLHRALRKATKLPVSTRCITLGTRCKVPSQCCRSSCKNGRCAPSPEEW

SEQ ID NO: 290 - protein, 31 aa, Conus viola
  PEPTIDE (1)..(31): Xaa at residue 29 and 30 is Glu or gamma- carboxy Glu; Xaa at residue 11, 26 and 28 is Pro or Hyp; Xaa at residue 31 is Trp or Bromo Trp
CITLGTRCKVXSQCCRSSCKNGRCAXSXXXX

SEQ ID NO: 291 - DNA, 372 bp, Conus viola
accaaaaccatcatcaaaatgaaactgacgtgtgtggtgatcgtcgccgtgctgctcctgacggcctgtcagctcattatagctggggactccagaggtacgcagttgcatcgtgccctgaggaaggccaccaaactctccgtgtcgactcgctgcaagagtagaggatcatcatgtcgtaggacttcgtatgactgctgcacgggttcttgcagaaatggtaaatgtggctgatccagcgcctgatcttcccccttctgtgctccatccttttctgcctgagtcctccttacctgagagtgggcatgaaccactcatcacctactccctggaagcttcagaggagctacattgaaataaaagccgcattgc

SEQ ID NO: 292 - protein, 71 aa, Conus viola
MKLTCVVIVAVLLLTACQLIIAGDSRGTQLHRALRKATKLSVSTRCKSRGSSCRRTSYDCCTGSCRNGKCG

SEQ ID NO: 293 - protein, 25 aa, Conus viola
  PEPTIDE (1)..(25): Xaa at residue 13 is Tyr, 125I-Tyr, mono- iodo-Tyr, di-iodo-Tyr, O-sulpho-Tyr or O-phospho-Tyr
CKSRGSSCRRTSXDCCTGSCRNGKC

SEQ ID NO: 294 - DNA, 380 bp, Conus viola
accaaaaccatcatcaaaatgaaactgacgtgtgtggcgatcgtcgccgtgctgctcctgacggcctgtcagctcattacagctgaagactccagaggtacgcatgagcatcttgccctgaagtcgacctccaaagtctccaagtcgactagctgcatggaagccagatcttattgcggacctgctactacgaaaatctgctgcgatttttgcagtccattcagcgatagatgtatgaacaatcccaacaattgatcttcccccttgtgtgctccatcttttctgcctgagtcctccttacctgagagtggtcatgaaccactcatcacctactcctctggaggcttcagaggagttacattgaaataaaagccgcatgc

SEQ ID NO: 295 - protein, 78 aa, Conus viola
MKLTCVAIVAVLLLTACQLITAEDSRGTHEHLALKSTSKVSKSTSCMEARSYCGPATTKICCDFCSPFSDRCMNNPNN

SEQ ID NO: 296 - protein, 36 aa, Conus viola
  PEPTIDE (1)..(36): Xaa at residue6  is Glu or gamma-carboxy Glu; Xaa at residue 13, 25 and 34 is Pro or Hyp; Xaa at residue 10 is Tyr, 125I-Tyr, mono-iodo-Tyr, di-iodo-Tyr, O-sulpho-Tyr or O- phospho-Tyr
STSCMXARSXCGXATTKICCDFCSXFSDRCMNNXNN

SEQ ID NO: 297 - DNA, 373 bp, Conus viola
accaaaaccatcatcaaaatgaaactgacgtgtgtggtgatcgtcgccgtgctgctcctgacggcctgtcagctcattacagctgaggactccagaggtacgcagttgcatcgtgccctgaggaagaccaccaaactctccttgtcgactcgctgcaagggtccaggagccatatgtataaggattgcgtataactgctgcaagtattcttgcggaaatggtaaatgtggctgatccagcgcctgatcttcccccttgtgtgctccatcctttttctgcctgagtcctccttacctgagagtggtcatgaaccactcatcacctactcctctggaggcttcagaggagctacattgaaataaaagccgcatgc

SEQ ID NO: 298 - protein, 71 aa, Conus viola
MKLTCVVIVAVLLLTACQLITAEDSRGTQLHRALRKTTKLSLSTRCKGPGAICIRIAYNCCKYSCGNGKCG

SEQ ID NO: 299 - protein, 25 aa, Conus viola
  PEPTIDE (1)..(25): Xaa at residue 3 is Pro or Hyp; Xaa at residue 13 and 18 is Tyr, 125I-Tyr, mono-iodo-Tyr, di-iodo-Tyr, O-sulpho- Tyr or O-phospho-Tyr
CKGXGAICIRIAXNCCKXSCGNGKC

SEQ ID NO: 300 - DNA, 353 bp, Conus viola
accaaaaccatcatcaaaatgaaactgacgtgtgtggtgatcgtcgccgtgctgttcctgacggcctgtcaattcatcacagctgatgactccagaagtacgcagaagcatcgtgccctgaggtcgaccaccaaacactttatgttgacttggtactgcacgccttatggaggacattgtggttattataatgactgctgcagtcatcaatgcaatataaacagaaataaatgtgagtagctgatccggcatctgatctgtgctcgccctaacctgagagtggtcatgaaccactcatcatctactcctctggaggcttcagaggagctacatggaaataaaagccgcattgc

SEQ ID NO: 301 - protein, 73 aa, Conus viola
MKLTCVVIVAVLFLTACQFITADDSRSTQKHRALRSTTKHFMLTWYCTPYGGHCGYYNDCCSHQCNINRNKCE

SEQ ID NO: 302 - protein, 28 aa, Conus viola
  PEPTIDE (1)..(28): Xaa at residue 28 is Glu or gamma-carboxy Glu; Xaa at residue 4 is Pro or Hyp; Xaa at residue 1, 5, 11 and 12 is Tyr, 125I-Tyr, mono-iodo-Tyr, di-iodo-Tyr, O-sulpho-Tyr or O- phospho-Tyr
XCTXXGGHCGXXNDCCSHQCNINRNKCX

SEQ ID NO: 303 - DNA, 294 bp, Conus pulicarius
ggatccatgaaactgacgtgcgtggtgattatcgccgtgctgttcctgacggcctgtcaactcattacagctgagacttactccagaggtaagcagatgcaccgtgctctgaggtcaactgacaaaaactccaagttgaccagggaatgcacacctccagatggagcttgtggtttacctacacactgctgcgggttttgcgatatggcaaacaacagatgtctgtaaagcgtctgatattccccttctgtgctctatcctctttggcctgagtcatccatacctgtgctcgag

SEQ ID NO: 304 - protein, 73 aa, Conus pulicarius
MKLTCVVIIAVLFLTACQLITAETYSRGKQMHRALRSTDKNSKLTRECTPPDGACGLPTHCCGFCDMANNRCL

SEQ ID NO: 305 - protein, 27 aa, Conus pulicarius
  PEPTIDE (1)..(27): Xaa at residue 1 is Glu or gamma-carboxy Glu; Xaa at residue 4, 5 and 12 is Pro or Hyp
XCTXXDGACGLXTHCCGFCDMANNRCL

SEQ ID NO: 306 - DNA, 294 bp, Conus pulicarius
ggatccatgaaactgacgtgcgtggtgattatcgccgtgctgttcctgacggcctgtcaactcattacagctgagacttactccagaggtaagcagatgcaccgtgctctgaggtcaactgacaaaaactcccagttgaccagggaatgcacacctccaggtggagcttgtggtttacctacacactgctgcgggttttgcgatatggcaaacaacagatgtctgtaaagcgtctgatattccccttctgtgctctatcctctttggcctgagtcatccatacctgtgctcgag

SEQ ID NO: 307 - protein, 73 aa, Conus pulicarius
MKLTCVVIIAVLFLTACQLITAETYSRGKQMHRALRSTDKNSQLTRECTPPGGACGLPTHCCGFCDMANNRCL

SEQ ID NO: 308 - protein, 27 aa, Conus pulicarius
  PEPTIDE (1)..(27): Xaa at residue 1 is Glu or gamma-carboxy Glu; Xaa at residue 4, 5 and 12 is Pro or Hyp
XCTXXGGACGLXTHCCGFCDMANNRCL

SEQ ID NO: 309 - DNA, 307 bp, Conus rattus
ggatccatgaaactgacgtgtgtggtgatcatcgccgtgctgttcctggcagcctgtcaacctgttacaactgagactttctccagaggtaaggagaagcgtcgtgctctgaggtcaactgacggcaactcccggttgaccagggcatgcacgcctgaaggtggagcctgtagtagtgggcgtcactgctgcggcttttgcgataacgtgtcccacacgtgctatggtgaaacaccatctctccactgatgtttccccttctgtgctctatcttcttttgcctgagtcatccatacctgtgctcgag

SEQ ID NO: 310 - protein, 80 aa, Conus rattus
MKLTCVVIIAVLFLAACQPVTTETFSRGKEKRRALRSTDGNSRLTRACTPEGGACSSGRHCCGFCDNVSHTCYGETPSLH

SEQ ID NO: 311 - protein, 34 aa, Conus rattus
  PEPTIDE (1)..(34): Xaa at residue 5 and 29 is Glu or gamma- carboxy Glu; Xaa at residue 4 and 31 is Pro or Hyp; Xaa at residue 27 is Tyr, 125I-Tyr, mono-iodo-Tyr, di-iodo-Tyr, O-sulpho-Tyr or O-phospho-Tyr
ACTXXGGACSSGRHCCGFCDNVSHTCXGXTXSLH

SEQ ID NO: 312 - DNA, 342 bp, Conus stercusmuscarum
  misc_feature (1)..(342): n may be any nucleotide
agatccatgaaactgacgtgcgtggtgatcgtcgccgtgctgctcctgacggcctgtcaactcatcacagctgatgactccagaggtacgcaggagcatcgtgccctgaggtcggacaccaaactccccatatcgactcgctgcaagggtaaaggagcatcatgtcataagactatgtatgactgctgcagcggttcctgcaccagaggtagatgtggctgatccagcgcctgatcttcccccttctgtgctctatccttttctgcctgagtcatcatacctgtgctcgagcgttactagtggatccgagctcggtaccaagcttggcgtaatcataaaanc

SEQ ID NO: 313 - protein, 71 aa, Conus stercusmuscarum
MKLTCVVIVAVLLLTACQLITADDSRGTQEHRALRSDTKLPISTRCKGKGASCHKTMYDCCSGSCTRGRCG

SEQ ID NO: 314 - protein, 25 aa, Conus stercusmuscarum
  PEPTIDE (1)..(25): Xaa at residue 13 is Tyr, 125I-Tyr, mono-iodo- Tyr, di-iodo-Tyr, O-sulpho-Tyr or O-phospho-Tyr
CKGKGASCHKTMXDCCSGSCTRGRC

SEQ ID NO: 315 - protein, 33 aa, Conus arenatus
QCSANGGSCTRHFHCCSLYCNKDSSVCVATSYP

SEQ ID NO: 316 - protein, 28 aa, Conus arenatus
TCNTPTEYCTLHRHCCSGYCHKTIQACS

SEQ ID NO: 317 - protein, 33 aa, Conus arenatus
QCTPNGGSCSRHFHCCSLYCNKSTGVCIATSYP

SEQ ID NO: 318 - protein, 33 aa, Conus arenatus; identical to SEQ ID NO: 317

SEQ ID NO: 319 - protein, 27 aa, Conus arenatus
ECTPPGGACGLPTHCCGFCDTANNRCL

SEQ ID NO: 320 - protein, 28 aa, Conus arenatus
TCNTPTEYCTLHQHCCSGHCHKTIQACA

SEQ ID NO: 321 - protein, 30 aa, Conus arenatus
QCSPIGGYCTLHIHCCSNHCIKPIGRCVAT

SEQ ID NO: 322 - protein, 30 aa, Conus arenatus
QCLPNGGYCTLHIHCCSDHCIKPIDRCVAT

SEQ ID NO: 323 - protein, 25 aa, Conus aurisiacus
CKGKGKPCSRISYNCCTGSCRSGKC

SEQ ID NO: 324 - protein, 32 aa, Conus aurisiacus
CMEAGSYCGSTTRICCGFCAYFGKKCIDYPSN

SEQ ID NO: 325 - protein, 25 aa, Conus aurisiacus
CKAKGKPCSRIAYNCCTGSCRSGKC

SEQ ID NO: 326 - protein, 26 aa, Conus aurisiacus
CASYGKPCGIDNDCCNACDPGRNICT

SEQ ID NO: 327 - protein, 36 aa, Conus bullatus
STSCMEAGSYCGPATTKICCDFCSPFSDRCMNNPNN

SEQ ID NO: 328 - protein, 31 aa, Conus bullatus
CITPGTRCKVPSQCCRGPCKNGRCTPSPSEW

SEQ ID NO: 329 - protein, 26 aa, Conus bullatus
CATYGKPCGIQNDCCNTCDPARRTCT

SEQ ID NO: 330 - protein, 25 aa, Conus bullatus
CKGPGASCIRIAYNCCKYSCRNGKC

SEQ ID NO: 331 - protein, 36 aa, Conus bullatus
STSCMAAGSYCGPATTNICCDFCSPFSDRCMKKPNN

SEQ ID NO: 332 - protein, 25 aa, Conus bullatus
CKSKGSSCHRTSYDCCTGSCRNGRC

SEQ ID NO: 333 - protein, 25 aa, Conus catus
CKSTGASCRRTSYDCCTGSCRSGRC

SEQ ID NO: 334 - protein, 25 aa, Conus catus
CQGRGASCRKTMYNCCSGSCNRGSC

SEQ ID NO: 335 - protein, 28 aa, Conus catus
CLPAGESCLFSRIRCCGTCSSVLKSCVS

SEQ ID NO: 336 - protein, 25 aa, Conus catus
CQGRGGPCTKAVFNCCSGSCNRGRC

SEQ ID NO: 337 - protein, 26 aa, Conus catus
CATYGKPCGIQNDCCNTCDPARKTCT

SEQ ID NO: 338 - protein, 25 aa, Conus catus
CRGRGGPCTKAMFNCCSGSCNRGRC

SEQ ID NO: 339 - protein, 33 aa, Conus caracteristicus; identical to SEQ ID NO: 315

SEQ ID NO: 340 - protein, 26 aa, Conus consors
CASYGKPCGIDNDCCNTCDPARKTCT

SEQ ID NO: 341 - protein, 25 aa, Conus consors
CKGTGKPCSRIAYNCCTGSCRSGKC

SEQ ID NO: 342 - protein, 36 aa, Conus consors
ATDCIEAGNYCGPTVMKICCGFCSPYSKICMNYPQN

SEQ ID NO: 343 - protein, 27 aa, Conus catus
CKGKGASCTRLMYDCCHGSCSSSKGRC

SEQ ID NO: 344 - protein, 25 aa, Conus consors
CKGKGASCHRTSYDCCTGSCNRGKC

SEQ ID NO: 345 - protein, 26 aa, Conus consors
CASYGKPCGIYNDCCNTCDPARKTCT

SEQ ID NO: 346 - protein, 25 aa, Conus consors
CKGTGKPCSRVAYNCCTGSCRSGKC

SEQ ID NO: 347 - protein, 35 aa, Conus consors
STSCMKAGSYCRSTTRTCCGYCAYFGKFCIDFPSN

SEQ ID NO: 348 - protein, 25 aa, Conus circumcisus
CKGKGASCRKTMYNCCSGSCSNGRC

SEQ ID NO: 349 - protein, 35 aa, Conus circumcisus
STSCMEAGSYCRSTTRTCCGYCSYFSKKCIDFPSN

SEQ ID NO: 350 - protein, 27 aa, Conus circumcisus
CKSKGAKCSRLMYDCCSGSCSRYSGRC

SEQ ID NO: 351 - protein, 35 aa, Conus circumcisus
STGCMKAGSYCRSTTRTCCGYCAYFGKKCIDYPSN

SEQ ID NO: 352 - protein, 28 aa, Conus dalli
SCTPPGGPCGYYNDCCSHQCNISRNKCE

SEQ ID NO: 353 - protein, 25 aa, Conus distans
  PEPTIDE (1)..(25): Xaa is Hyp
CEDXGEXCGSDHSCCGGSCNHNVCA

SEQ ID NO: 354 - protein, 27 aa, Conus ermineus
PCKPKGRKCFPHQKDCCNKTCTRSKCP

SEQ ID NO: 355 - protein, 27 aa, Conus ermineus
ACWSSGTPCGTDSLCCGGCNVSKSKCN

SEQ ID NO: 356 - protein, 27 aa, Conus geographus
CKSPGSSCSPTSYNCCRSCNPYAKRCY

SEQ ID NO: 357 - protein, 29 aa, Conus geographus
CKSPGTPCSRGMRDCCTPCLLYSNKCRRY

SEQ ID NO: 358 - protein, 30 aa, Unknown
  unknown Conus species
CLSPGSRCHKTMRNCCTSCSSYKGKCRPRK

SEQ ID NO: 359 - protein, 27 aa, Unknown
  unknown Conus species
CKPPGRKCLNRKNECCSKFCNEHLHMC

SEQ ID NO: 360 - protein, 26 aa, Unknown
  unknown Conus species
CKPPRRKCLKIKDKCCNFCNTHLNMC

SEQ ID NO: 361 - protein, 28 aa, Unknown
  unknown Conus species
CAGPGTICPNRVCCGYCSKRTHLCHSRT

SEQ ID NO: 362 - protein, 27 aa, Conus laterculatus
KCWPSGSYCRANSKCCSGCDRNRNKCY

SEQ ID NO: 363 - protein, 27 aa, Conus laterculatus
CLPPGSYCKATTEVCCSSCLQFAQICS

SEQ ID NO: 364 - protein, 30 aa, Conus lynceus
CKSPGSPCSVTSYNCCTFCSSYTKKCRASL

SEQ ID NO: 365 - protein, 28 aa, Conus lynceus
CAGPGAICPNRVCCGYCSKRTHLCHSRT

SEQ ID NO: 366 - protein, 27 aa, Conus lynceus
ACWSSGTPCGTDSLCCGGCNVSKSKCN

SEQ ID NO: 367 - protein, 27 aa, Conus lynceus
KCWSPGTYCRAHSKCCRGCDQNRNKCY

SEQ ID NO: 368 - protein, 29 aa, Conus laterculatus
CKSPGSSCSVSMRNCCTSCNSRTKKCTRR

SEQ ID NO: 369 - protein, 27 aa, Conus laterculatus
TCWPSGTACGIDSNCCSGCNVSRSKCN

SEQ ID NO: 370 - protein, 27 aa, Conus laterculatus
KCWPSGSYCRANSKCCSGCDRNRSKCN

SEQ ID NO: 371 - protein, 37 aa, Conus leopardus
SLFECAPSGGRCGFLKSCCEGYCDGESTSCVSGPYSI

SEQ ID NO: 372 - protein, 30 aa, Conus leopardus
WPLDCTAPSQPCGYFPRCCGHCDVRRVCTS

SEQ ID NO: 373 - protein, 31 aa, Conus leopardus
CMSPGGICGDFGDCCEICNVYGICVSDLPGI

SEQ ID NO: 374 - protein, 27 aa, Conus leopardus
YCAPPGGACGFFDHCCGYCETFYNTCR

SEQ ID NO: 375 - protein, 25 aa, Conus magus
CKGTGKPCSRIAYNCCTGSCRSGKC

SEQ ID NO: 376 - protein, 26 aa, Conus magus
CASYGKPCGIYNDCCNTCDPARKTCT

SEQ ID NO: 377 - protein, 27 aa, Conus miles
CNDRGGGCSQHPHCCGGTCNKLIGVCL

SEQ ID NO: 378 - protein, 25 aa, Conus monachus
CKSTGKSCSRIAYNCCTGSCRSGKC

SEQ ID NO: 379 - protein, 25 aa, Conus monachus
CKGKGSSCSRTMYNCCTGSCNRGKC

SEQ ID NO: 380 - protein, 35 aa, Conus obscurus
SPPCMKGGSSCRGTTGVCCGFCSDFGYKCRDYPQN

SEQ ID NO: 381 - protein, 28 aa, Conus obscurus
CLPDGTSCLFSRIRCCGTCSSILKSCVS

SEQ ID NO: 382 - protein, 27 aa, Conus purpurascens
  PEPTIDE (1)..(27): Xaa is Hyp
XCKTXGRKCFXHQKDCCGRACIITICP

SEQ ID NO: 383 - protein, 26 aa, Conus purpurascens
  PEPTIDE (1)..(26): Xaa at residue 5 is Hyp; Xaa at residue 12 is gamma-carboxy-Glu
SCKLXGAYCNAXDYDCCLRCKVGGTC

SEQ ID NO: 384 - protein, 27 aa, Conus purpurascens
PCKKTGRKCFPHQKDCCGRACIITICP

SEQ ID NO: 385 - protein, 30 aa, Conus pulicarius
QCSPNGGSCSRHFHCCSLYCNKNTGVCIAT

SEQ ID NO: 386 - protein, 27 aa, Conus pulicarius
ECTPPDGACGLPTHCCGFCDMANNRCL

SEQ ID NO: 387 - protein, 27 aa, Conus pulicarius
ECTPPGGACGLPTHCCGFCDMANNRCL

SEQ ID NO: 388 - protein, 28 aa, Conus radiatus
HGCKPLKRRCFNDKECCSKFCNSVRKQC

SEQ ID NO: 389 - protein, 28 aa, Conus radiatus
RGCKPLKRRCFNDKECCSKFCNSVRNQC

SEQ ID NO: 390 - protein, 27 aa, Conus rattus
CNARNDGCSQHSQCCSGSCNKTAGVCL

SEQ ID NO: 391 - protein, 27 aa, Conus rattus
CNARNSGCSQHPQCCSGSCNKTAGVCL

SEQ ID NO: 392 - protein, 27 aa, Conus rattus
CNARNSGCSQHPQCCSGSCNKTLGVCL

SEQ ID NO: 393 - protein, 34 aa, Conus rattus
ACTPEGGACSSGRHCCGFCDNVSHTCYGETPSLH

SEQ ID NO: 394 - protein, 36 aa, Conus striatus
ATDCIEAGNYCGPTVMKICCGFCSPYSKICMNYPKN

SEQ ID NO: 395 - protein, 26 aa, Conus striatus
CKLKGQSCRRTMYDCCSGSCGRRGKC

SEQ ID NO: 396 - protein, 25 aa, Conus striatus
CKAAGKSCSRIAYNCCTGSCRSGKC

SEQ ID NO: 397 - protein, 26 aa, Conus striatus
CESYGKPCGIYNDCCNACDPAKKTCT

SEQ ID NO: 398 - protein, 27 aa, Conus stercusmuscarum
CKSKGAKCSRLMYDCCSGSCSGYTGRC

SEQ ID NO: 399 - protein, 35 aa, Conus stercusmuscarum
TTSCMQAGSYCGSTTRICCGYCAYFGKKCIDYPSN

SEQ ID NO: 400 - protein, 26 aa, Conus stercusmuscarum
CASYGKPCGIDNDCCNACDPARNICT

SEQ ID NO: 401 - protein, 26 aa, Conus stercusmuscarum
CVSYGKPCGIDNDCCNACDPARNICT

SEQ ID NO: 402 - protein, 25 aa, Conus stercusmuscarum
CKGKGASCHKTMYDCCSGSCTRGRC

SEQ ID NO: 403 - protein, 25 aa, Conus striolatus
CKGKGASCLRTAYDCCTGSCNRGRC

SEQ ID NO: 404 - protein, 24 aa, Conus striolatus
CRPSGSNCGNISICCGRCVNRRCT

SEQ ID NO: 405 - protein, 35 aa, Conus striolatus
STSCMKAGSYCVATTRICCGYCAYFGKICIDYPKN

SEQ ID NO: 406 - protein, 28 aa, Conus textile
YCTPHGGHCGYHNDCCSHQCNINRNKCE

SEQ ID NO: 407 - protein, 31 aa, Conus viola
CITLGTRCKVPSQCCRSSCKNGRCAPSPEEW

SEQ ID NO: 408 - protein, 25 aa, Conus viola
CKSRGSSCRRTSYDCCTGSCRNGKC

SEQ ID NO: 409 - protein, 36 aa, Conus viola
STSCMEARSYCGPATTKICCDFCSPFSDRCMNNPNN

SEQ ID NO: 410 - protein, 25 aa, Conus viola
CKGPGAICIRIAYNCCKYSCGNGKC

SEQ ID NO: 411 - protein, 28 aa, Conus viola
YCTPYGGHCGYYNDCCSHQCNINRNKCE

SEQ ID NO: 412 - protein, 27 aa, Conus textile
CTPYGGHCGYNHDCCSHQCNINRNKCE

SEQ ID NO: 413 - protein, 26 aa, Conus tulipa
  PEPTIDE (1)..(26): Xaa is Hyp
CKSWGSXCSXTSTNCCWSCSPYRKKC